(19) United States
(12) Patent Application Publication
(10) Pub. No.: US 2020/0405841 A1
(43) Pub. Date: Dec. 31, 2020

(54) Immunogenic Composition

(71) Applicant: GLAXOSMITHKLINE BIOLOGICALS SA, Rixensart (BE)

(72) Inventors: Nathalie Isabelle Devos, Rixensart (BE); Vincent DEWAR, Rixensart (BE); Philippe Vincent HERMAND, Rixensart (BE); Silvia ROSSI PACCANI, Siena (IT); Vincent WEYNANTS, Wavre (BE)

(73) Assignee: GLAXOSMITHKLINE BIOLOGICALS SA, Rixensart (BE)

(21) Appl. No.: 16/978,870

(22) PCT Filed: Mar. 6, 2019
(86) PCT No.: PCT/EP2019/055496

§ 371 Date: Sep. 8, 2020

(30) Foreign Application Priority Data
Mar. 8, 2018 (GB) 1803692.1

(51) Int. Cl. A61K 39/104; C07K 14/785; C07K 14/435; C07K 14/21; C07K 16/12

(52) U.S. Cl. CPC A61K 39/1045; C07K 14/785; C07K 14/435; C07K 14/212; A61K 2039/55516; C07K 2317/33; C07K 2317/20; C07K 2317/565; C07K 16/1214

(57) ABSTRACT

The present invention relates to the field of immunogenic compositions and vaccines and the use of such compositions in medicine. More particularly, it relates to immunogenic fragments of UspA2 comprising an epitope and immunogenic compositions and vaccines comprising said fragments, for use in preventing or treating an infection, disease or condition caused by heterologous strain(s) of Moraxella catarrhalis. The invention further relates to immunogenic compositions and vaccines comprising an immunogenic fragment wherein said fragment comprises an epitope with cross-bactericidal activity.

### TECHNICAL FIELD

The present invention relates to the field of immunogenic compositions and vaccines and the use of such compositions in medicine. More particularly, it relates to immunogenic fragments of UspA2 comprising an epitope and immunogenic compositions comprising said fragments, for use in preventing or treating an infection, disease or condition caused by heterologous strain(s) of Moraxella catarrhalis.

### BACKGROUND

Ubiquitous surface protein (Usp) A1 and UspA2 are adhesion and autotransporter proteins. They play a role in serum resistance and other virulence mechanisms.

Ubiquitous surface protein A2 (UspA2) is a trimeric autotransporter that appears as a lollipop-shared structure in electron micrographs (1). It is composed of a N-terminal head, followed by a stalk which ends by an amphipathic helix and a C-terminal membrane domain (1). UspA2 contains a very well conserved domain (2) which is recognized by a monoclonal antibody (17C7) that was shown protective upon passive transfer in a mouse Moraxella catarrhalis challenge model (3). UspA1 and UspA2 can be distinguished by differences in amino acid sequences within the head and membrane-spanning regions, yet they share homology within the stalk region. UspA2H is a “hybrid” protein containing a head region (N-terminal) similar to that of UspA1 while having the UspA2-like C-terminal region. Meanwhile, UspA2V is a variant with the Carcino-Embryonic Antigen-Related Cell Adhesion Molecule 1 (CEACAM1)-binding region of UspA1.

UspA2 is heat modifiable with a predicted molecular weight of 60 kDa, but it appears above 200 kDa after denaturation in SDS-PAGE (4). UspA2 has been shown to interact with host structures and extracellular matrix proteins like fibronectin (5) and laminin (6) suggesting it can play a role at an early stage of Moraxella catarrhalis infection.

UspA2 also seems to be involved in the ability of Moraxella catarrhalis to resist the bactericidal activity of normal human serum (7). It (i) binds the complement inhibitor C4 bp, enabling Moraxella catarrhalis to inhibit the classical complement system, (ii) prevents activation of the alternative complement pathway by absorbing C3 from serum and (iii) interferes with the terminal stages of the complement system, the Membrane Attack Complex (MAC), by binding the complement regulator protein vitronectin (8).

Moraxella catarrhalis is an important and common respiratory pathogen that has been associated with increased risk of exacerbations in chronic obstructive pulmonary disease (COPD) in adults (9). COPD is a leading cause of morbidity and mortality worldwide.

Chronic Obstructive Pulmonary Disease (COPD), a common preventable disease, is characterised by persistent airflow limitation that is usually progressive. The airflow limitation is associated with an enhanced chronic inflammatory response in the airways and lungs to noxious particles of gases. It is a multi-component disease that manifests as an accelerated decline in lung function, with symptoms such as breathlessness on physical exertion, deteriorating health status and exacerbations.

Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication (9). AECOPD increases morbidity and mortality, leading to faster decline in lung function, poorer functional status (10). The lungs are known to be colonised with different strains of bacteria (11, 12). In COPD patients, acquisition of new bacterial strains is believed to be an important cause of AECOPD (13). Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) (14-16).

Recently the utility of UspA as a cross-protective antigen was challenged due to sequence heterogeneity and varying structures (17). This results in different phenotypes and divergent functions in terms of interacting with host targets among strains and clinical isolates. As a result, the suitability of UspA antigens as vaccine targets was questioned. There is a need to provide further immunogenic compositions for use in preventing or treating an infection, disease or condition caused by Moraxella catarrhalis.

### SUMMARY OF THE INVENTION

The present invention provides immunogenic fragments of UspA2 comprising an epitope and immunogenic compositions comprising said fragments, for use in preventing or treating an infection, disease or condition caused by heterologous strain(s) of Moraxella catarrhalis. More particularly the present invention provides immunogenic fragments of UspA2 for use in preventing or treating an infection disease or condition caused by heterologous strain(s) M. catarrhalis comprising an epitope with cross-bactericidal activity.

Accordingly, in a first aspect of the invention there is provided an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids (e.g. less than 445 amino acids, less than 300 amino acids, less than 150 amino acids etc.)

According to a further aspect of the invention, there is provided an antigen binding protein which binds to an epitope within a consensus sequence of YNELQD-[A/Q]-YA-[QK/KQ]-QTE (SEQ ID NO: 64), e.g. SEQ ID NO: 60, 61, 62 or 63.

According to a further aspect of the invention, there is provided an antigen binding protein comprising any one or a combination of CDRs selected from CDRH1, CDRH2, CDRH3 from SEQ ID NO: 82, and/or CDRL1, CDRL2, CDRL3 from SEQ ID NO: 84; or (ii) a CDR variant of (i), wherein the variant has 1, 2, or 3 amino acid modifications in each CDR, which is able to bind to an epitope within the consensus sequence of SEQ ID NO: 64 and promote bactericidal activity

According to a further aspect of the invention, there is provided an antibody that binds to UspA2, and competes for binding to the consensus sequence SEQ ID NO: 64 with a reference the antibody with a VH region comprising SEQ ID NO: 82 and a VL region comprising SEQ ID NO: 84.

According to a further aspect of the invention, there is provided an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 continuous amino acids (e.g. less than 449 amino acids, less than 300 amino acids, less than 150 amino acids etc.) for use in eliciting cross-bactericidal activity against heterologous strain(s) of M. catarrhalis.

According to a further aspect of the invention there is provided an immunogenic composition comprising the immunogenic fragments of the invention.

According to a further aspect of the invention, there is provided a vaccine comprising the immunogenic fragment or the immunogenic composition of the invention.

According to a further aspect of the invention, there is provided the vaccine of the invention for use in treating or preventing an infection, disease or condition caused by M. catarrhalis in a mammal, particularly a human.

According to a further aspect of the invention, there is provided the vaccine of the invention for use in the treatment or prevention of acute exacerbations of acute otitis media, pneumonia and/or chronic obstructive pulmonary disease (AECOPD).

According to a further aspect of the invention there is provided a method of treatment or prevention of an infection, disease or condition caused by M. catarrhalis, in a subject in need thereof comprising administering to said subject a therapeutically effective amount of an immunogenic composition of the invention or vaccine of the invention.

According to a further aspect of the invention there is provided a method of treatment or prevention of acute exacerbations of chronic obstructive pulmonary disease (AECOPD), pneumonia and/or otitis media in a subject in need thereof comprising administering to said subject a therapeutically effective amount of an immunogenic composition of the invention or vaccine of the invention.

According to a further aspect of the invention there is provided the immunogenic fragment, immunogenic composition or vaccine of the invention for use in the manufacture of a medicament for the treatment or prevention of an infection, disease or condition caused by M. catarrhalis.

According to a further aspect of the invention there is provided the immunogenic fragment, immunogenic composition or vaccine of the invention for use in the manufacture of a medicament for the treatment of pneumonia, otitis media and/or AECOPD.

### DESCRIPTION OF FIGURES

FIG. 1: Coiled coil periodicity in UspA2 str. 25238. Positions a and d of canonical heptad repeats and h of undecad repeats are indicated above the sequence.

FIG. 2: Sequence alignment between UspA2 str. 25238 and the artificial template built with UspA1 x-ray coordinated. Heptad repeats are highlighted in grey. Despite the low sequence similarity in the first half of the alignment, the sequence periodicity was conserved between UspA2 and the template.

FIG. 3: Three-dimensional model of the 301-470 (SEQ ID NO: 1) region of UspA2. Atoms of side chains of 308-321, 343-356 and 378-381 are represented as sphere and indicated by arrows. Details are shown for 308-321 and 378-356.

FIG. 4A: Electron micrograph demonstrating that at molar ratios of 1:1 (UspA2 trimer: FHUSPA2/10), FHUSPA2/10 binds to the primary motif.

FIG. 4B: Electron micrograph demonstrating that at a high concentrations i.e. molar ratio of 1:3 (UspA2 trimer: FHUSPA2/10), FHUSPA2/10 binds secondary motifs at a distance of 5 nm from primary motif

FIG. 4C: Electron micrograph demonstrating that at a high concentrations i.e. molar ratio of 1:3 (UspA2 trimer: FHUSPA2/10), FHUSPA2/10 can bind secondary motifs at a distance of 10 nm from primary motif

FIG. 4D: Single FHUSPA2/10 antibodies are able to bind to the primary motifs of multiple UspA2 trimers.

FIG. 4E: FHUSPA2/10 can promote UspA2 intermolecular bridging.

FIG. 4F: UspA2 is a highly elongated and stable structure. UspA2 is a homotrimer composed of a stalk decorated by a small head.

FIG. 5: UspA2 peptide map used for HDX-MS epitope mapping (132 pepsin peptides used). Taking into consideration the repeated regions highlighted, 97.4% of the full-length sequence is covered.

FIG. 6: The deuterium incorporation of 132 peptides generated from the antigen (UspA2 SEQ ID NO 2) under its free or mAb-bound form with a molar ratio of 1:0.33.

FIG. 7: Increasing the amount of mAb compared to the protein (protein monomer/antibody molar ratio of 1:1), results in a binding also in the repeated regions.

### DETAILED DESCRIPTION

### Terminology

To facilitate review of the various embodiments of this disclosure, the following explanations of terms are provided. Additional terms and explanations are provided in the context of this disclosure.

Unless otherwise explained or defined herein, all technical and scientific terms used herein have the same meaning as commonly understood by one of ordinary skill in the art to which this disclosure belongs. For example, definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

All references or patent applications cited within this patent specification are incorporated by reference herein.

Amino acids refers to an amino acid selected from the group consisting of alanine (ala, A), arginine (arg, R), asparagine (asn, N), aspartic acid (asp, D), cysteine (cys, C), glutamine (gin, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), valine (val, V).

Numerical limitations given with respect to concentrations or levels of a substance, such as an antigen may be approximate. Thus, where a concentration is indicated to be (for example) approximately 200 pg, it is intended that the concentration includes values slightly more or slightly less than (“about” or “˜”) 200 pg.

Although methods and materials similar or equivalent to those described herein can be used in the practice or testing of this disclosure, suitable methods and materials are described below.

The abbreviation, “e.g.” is derived from the Latin exempli gratia, and is used herein to indicate a non-limiting example. Thus, the abbreviation “e.g.” is synonymous with the term “for example.”

As used herein, the term “epitope” refers to the portion of a macromolecule (antigen) which is specifically recognised by a component of the immune system e.g. an antibody or a T-cell antigen receptor. The term epitope may refer to that portion of the antigen that makes contact with a particular binding domain of the antigen binding protein. An epitope may be linear or conformational/discontinuous. A conformational or discontinuous epitope comprises amino acid residues that are separated by other sequences, i.e. not in a continuous sequence in the antigen's primary sequence. Although the residues may be from different regions of the peptide chain, they are in close proximity in the three-dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. An epitope may reside within the consensus sequence of the invention.

As used herein, the term “confers” means to give/provide a function from one integer to another. For example, wherein the epitope of the invention “confers” (i.e. is the integer which gives/provides) cross-bactericidal activity to the immunogenic fragment of the invention.

As used herein, the term “heterologous strain(s) of M. catarrhalis” refers to strain(s) of M. catarrhalis which are different from the M. catarrhalis strain from which the immunogenic fragment used to immunize the subject against M. catarrhalis was derived (e.g. different from SEQ ID NO: 1). More particularly heterologous strain(s) of M. catarrhalis refers to strain(s) of M. catarrhalis which may be substantially different (i.e. with 40-99% identity to strain used to immunize the subject against M. catarrhalis, i.e. 40-95%, 45-85%, 45-80%, 50-90%, 55-85%, 60-99%, 63-95% etc) from the M. catarrhalis strain used to immunize the subject against M. catarrhalis (e.g. SEQ ID NO:1). Heterologous strain(s) of M. catarrhalis may express different UspA1 and UspA2 variants. For example, for an immunogenic fragment of UspA2 (e.g. SEQ ID NO: 1) a heterologous strain(s) of M. catarrhalis may be one which expresses a variant of UspA2 e.g. UspA2H or UspA2V. For example, for an immunogenic fragment of UspA2 (e.g. SEQ ID NO: 1) a heterologous strain(s) of M. catarrhalis may be one which is UspA1 positive/UspA2 negative (i.e. UspA1*/UspA2-) or a strain which comprises UspA1 and an UspA2 variant e.g. UspA2H or UspA2V (e.g. UspA2H positive/UspA2 negative or UspA2V positive/UspA2 negative). For example, for an immunogenic fragment of UspA2 (E.g. SEQ ID NO: 1) a heterologous strain(s) of M. catarrhalis may be strains with an UspA2 sequence according to any of SEQ ID NOS: 2-28 or a sequence at least 80% identical to any of SEQ ID NO: 2-28.

A “subject” as used herein is a mammal, including humans, non-human primates, and non-primate mammals. In one aspect, a subject is a human.

As used herein, “immune response” means the sequence of events occurring at the molecular, cellular or tissue level (i.e. at any level of biological organisation) in response to an antigen. In the context of the present disclosure, “immune response” may be the sequence of cellular (cell mediated) and/or humoral (antibody mediated) events occurring in response to an antigen (e.g. antigens on the surface of bacteria, viruses, fungi etc. or in response to antigens presented in the form of an immunogenic fragment, immunogenic composition or vaccine).

As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens (compared to the immune response obtained in the absence of adjuvant).

As used herein the term “protect or treat” in the context of infection, diseases or conditions caused by M. catarrhalis means either to protect via prophylaxis or treat via administration post-infection any M. catarrhalis causing symptom, effect or phenotype. Protection and treatment of an infection, disease or condition caused by M. catarrhalis includes amelioration of M. catarrhalis related effects.

As used herein, the term “effective amount,” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

As used herein the term “amino acid modification” involves any modification to the DNA, RNA or protein which alters the sequence of the polypeptide. This may include (but is not limited to) polymorphisms, DNA mutations (including single nucleotide polymorphisms), post-translational modifications etc.

As used herein, the term “conservative amino acid substitution” involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics like those of a parental polypeptide.

Amino acid substitutions may be conservative or non-conservative. In some embodiments, amino acid substitution is conservative. Substitutions, deletions, additions or any combination thereof may be combined in a single variant so long as the variant is an immunogenic polypeptide.

Embodiments herein relating to “vaccine compositions” of the invention are also applicable to embodiments relating to “immunogenic compositions” of the invention, and vice versa.

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 10 residues, (e.g. 1 to 7 residues, 1 to 6 residues, or 1 to 4 residues) are inserted at any one site within the protein molecule.

As used herein “signal peptide” refers to a short (less than 60 amino acids, for example, 3 to 60 amino acids) polypeptide present on precursor proteins (typically at the N terminus), and which is typically absent from the mature protein. The signal peptide (sp) is typically rich in hydrophobic amino acids. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide.

As used herein, the term “bactericidal” is the ability of an antibody (including a vaccine-induced antibody) to kill bacteria. It is measured using the serum bactericidal antibody (SBA) assay as described in Example 4 which measures complement mediated killing via antibodies. The SBA assay requires active complement which in Example 4 is provided in the form of exogenous complement (from Baby Rabbit serum). Complement can be either from a human or from another species. As used herein the term “cross-bactericidal” is the ability of an antibody to kill bacteria which expresses an antigen with differing levels of sequence identity to the antigen used to generate the antibody.

As used herein the term “eliciting cross-bactericidal activity” relates to the use of an antibody or immunogenic fragment (when used to produce antibodies as a result of administration of an immunogenic composition or vaccine) which are able to kill bacteria which express an antigen with differing levels of sequence identity to the antigen used to generate said antibody or immunogenic fragment.

As used herein the term “cross-protection” refers to the ability of an immunogenic fragment or antibody to protect a subject from disease (when administered prophylactically) following infection by a strain of pathogen which expresses an antigen with differing levels of sequence identity to the antigen used to generate said immunogenic fragment or antibody.

As used herein the term “antigen binding protein” refers to antibodies and other protein constructs, such as domains, which are capable of binding to an antigen (for example UspA2).

As used herein the term “antibody” is used in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see (18)). Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

As used herein, the term “immunogenic fragment” is a portion of an antigen smaller than the whole, that can elicit a humoral and/or cellular immune response in a host animal, e.g. human, specific for that fragment. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40 or 50 contiguous amino acids of the full-length sequence. Fragments may be readily modified by adding or removing 1, 2, 3, 4, 5, 6, 7, 8, 9, 10, 20, 30, 40 or 50 amino acids from either or both of the N and C termini. Furthermore, fragments may be modified by adding 1, 2, 3, 4, 5, 6, 7, 8, 9 or 10 terminal histidine residues (histidine tags) at either the N- or the C-terminus of the protein.

As used herein the term “fragment” refers to a sequence that is a subset of another sequence. The term is used to refer to a part or portion of an intact or complete wild-type polypeptide but which comprise fewer amino acid residues than the intact or complete wild-type polypeptide. Thus, the term refers to truncated or shorter amino acid sequences corresponding to one or more regions of a wild-type or reference polypeptide and it is to be understood that as used herein, the term fragment excludes reference to the full-length or wild-type polypeptide sequence. One example of a fragment is an epitope sequence. A fragment or subsequence of an amino acid sequence can be any number of residues less than that found in the naturally occurring, or reference, polypeptide. However, it will be clear to one skilled in the art that, in the context of the present invention, any such immunogenic fragments must be capable of eliciting an immune response against the full-length polypeptide, particularly an immune response that results in the formation of antibodies capable of binding to the full-length polypeptide. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide.

It will be clear to those skilled in the art that, whilst such fragments are truncated or shorter fragments of a reference sequence, such fragments may be modified to comprise additional sequences not found in the reference polypeptide, for example, to form fusion polypeptides, include ‘tag’ sequences such as His tags or Glutathione S-transferase (GST) tags, linker sequences and the like. Thus, in such modified fragments the amino group of the N terminal amino acid of the fragment is not linked by a peptide bond to the carboxyl group of an amino acid to which it would be linked in the reference polypeptide from which it is derived and/or the carboxyl group of the C terminal amino acid of the fragment is not linked by a peptide bond to the amino group of an amino acid to which it would be linked in the reference polypeptide from which it is derived.

As used herein “UspA2” means Ubiquitous surface protein A2 from Moraxella catarrhalis. UspA2 may consist of or comprise the amino acid sequence of SEQ ID NO: 1 from ATCC 25238.

(SEQ ID NO: 1)
MKTMKLLPLKIAVTSAMIIGLGAASTANAQAKNDITLEDLPYLIKKIDQNE
LEADIGDITALEKYLALSQYGNILALEELNKALEELDEDVGWNQNDIANLE
DDVETLTKNQNALAEQGEAIKEDLQGLADFVEGQEGKILQNETSIKKNTQR
NLVNGFEIEKNKDAIAKNNESIEDLYDFGHEVAESIGEIHAHNEAQNETLK
GLITNSIENTNNITKNKADIQALENNVVEELFNLSGRLIDQKADIDNNINN
IYELAQQQDQHSSDIKTLKKNVEEGLLELSGHLIDQKTDIAQNQANIQDLA
TYNELQDQYAQKQTEAIDALNKASSENTQNIEDLAAYNELQDAYAKQQTEA
IDALNKASSENTQNIEDLAAYNELQDAYAKQQTEAIDALNKASSENTQNIA
KNQADIANNINNIYELAQQQDQHSSDIKTLAKASAANTDRIAKNKADADAS
FETLTKNQNTLIEKDKEHDKLITANKTAIDANKASADTKFAATADAITKNG
NAITKNAKSITDLGTKVDGFDSRVTALDTKVNAFDGRITALDSKVENGMAA
QAALSGLFQPYSVGKFNATAALGGYGSKSAVAIGAGYRVNPNLAFKAGAAI
NTSGNKKGSYNIGVNYEF

as well as sequences with at least or exactly 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% 99.9% or 100% identity, over the entire length, to SEQ ID NO: 1.

Alternatively, UspA2 may consist of or comprise any of amino acid sequences SEQ ID NO: 1-SEQ ID NO: 38 or SEQ ID NO: 68-75. Strains of M. catarrhalis used in Example 4 showed UspA2 sequence identity of 57.3% or higher in comparison to SEQ ID NO: 1.

UspA2 as described in SEQ ID NO: 1 contains a signal peptide (for example, amino acids 1 to 29 of SEQ ID NO: 1), a laminin binding domain (for example, amino acids 30 to 177 of SEQ ID NO: 1), a fibronectin binding domain (for example, amino acids 165 to 318 of SEQ ID NO: 1) (5), a C3 binding domain (for example, amino acids 30 to 539 of SEQ ID NO: 1 (19), or a fragment of amino acids 30 to 539 of SEQ ID NO: 1, for example, amino acids 165 to 318 of SEQ ID NO: 1 (20), an amphipathic helix (for example, amino acids 519 to 564 of SEQ ID NO: 1 or amino acids 520-559 of SEQ ID NO:1, identified using different prediction methods) and a C terminal anchor domain (for example, amino acids 576 to 630 amino acids of SEQ ID NO: 1 (21).

UspA2 amino acid differences have been described for various Moraxella catarrhalis species. Furthermore, both conserved regions and regions of significant amino acid diversity have been reported across M. catarrhalis strains. See for example, (4, 21, 22).

UspA2 may consist of or comprise an amino acid sequence that differs from SEQ ID NO. 1 at any one or more amino acid selected from the group consisting of: AA (amino acid) 30 to 298, AA 299 to 302, AA 303 to 333, AA 334 to 339, AA 349, AA 352 to 354, AA 368 to 403, AA 441, AA 451 to 471, AA 472, AA474 to 483, AA 487, AA 490, AA 493, AA 529, AA 532 or AA 543. UspA2 may consist of or comprise an amino acid sequence that differs from SEQ ID NO: 1 in that it contains at least one amino acid insertion in comparison to SEQ ID NO. 1. UspA2 may consists of or comprise an amino acid sequence that differs from SEQ ID NO. 1 at any one of the amino acid differences in SEQ ID NO: 2 through SEQ ID NO: 38. For example, SEQ ID NO. 1 may contain K instead of Q at amino acid 70, Q instead of G at amino acid 135 and/or D instead of N at amino acid 216.

UspA2 may be UspA2 from M. catarrhalis strain ATCC (a US registered trademark) 25238™, American 2933. American 2912, American 2908, Finnish 307, Finnish 353, Finnish 358, Finnish 216, Dutch H2, Dutch F10, Norwegian 1, Norwegian 13, Norwegian 20, Norwegian 25, Norwegian 27, Norwegian 36, BC5SV, Norwegian 14, Norwegian 3, Finish 414, Japanese Z7476, Belgium Z7530, German Z8063, American 012E, Greek MC317, American V1122, American P44, American V1171, American TTA24, American 035E, American SP12-6, American SP12-5, Swedish BC5, American 7169, Finnish FIN2344, American V1118, American V1145 or American V1156. UspA2 may be UspA2 as set forth in any of SEQ ID NO: 1-SEQ ID NO: 38. UspA2 may be UspA2 which has been isolated from human subjects such as those in Example 4 which were isolated in the AERIS study (a clinical study wherein strains of M. catarrhalis were isolated from human subjects with AECOPD, see reference (68)).

UspA2 may be UspA2 from another source which corresponds to the sequence of UspA2 in any one of SEQ ID NO: 1-SEQ ID NO: 38. Corresponding UspA2 sequences may be determined by one skilled in the art using various algorithms. For example, the Gap program or the Needle program may be used to determine UspA2 sequences corresponding to any one of SEQ ID NO: 1-SEQ ID NO: 38. UspA2 may be a sequence with at least 80% identity, over the entire length, to any of SEQ ID NO: 1-SEQ ID NO: 38. Furthermore UspA2 may be detoxified by either amino acid modification or chemical methods which are known in the art.

Fragments of the UspA2 protein can be tested for functionality using the Serum Bactericidal Assay (as described in Example 4 herein). Inclusion of a negative control strain (i.e. one which is UspA-null) confirms that any response observed is UspA-dependent. As an alternative the skilled person may test the functionality of an UspA2 fragment in vivo for example using techniques referred to in (23) e.g. Mouse model of lung colonization (Example 8 of ref 23), Lung Challenge Model (Example 10 of ref 23) or Immunogenicity in Mice (Example 11-13 of ref 23). Analysis of UspA2 specific Anti-IgG antibodies can be performed by ELISA as described in Example 11 (page 84) of reference 23.

Reference to UspA1 herein may be UspA1 of SEQ ID NO: 54 (with signal peptide) or SEQ ID NO: 55 (without signal peptide) or a sequence with at least 60% similarity to SEQ ID NO: 54 or 55. Reference to UspA2H herein may be UspA2H of SEQ ID NO: 56 (with signal peptide) or SEQ ID NO: 57 (without signal peptide) or a sequence with at least 60% similarity to SEQ ID NO: 56 or 57. Reference to UspA2V herein may be UspA2V of SEQ ID NO: 58 (with signal peptide) or SEQ ID NO: 59 (without signal peptide) or a sequence with at least 60% similarity to SEQ ID NO: 58 or 59.

Recently, the utility of UspA as a cross-protective antigen was challenged due to sequence heterogeneity (17). A need for an UspA2 antigen with cross-bactericidal and/or cross-protective properties exits. Such cross-protection would reduce the risk of escape mutants if the UspA2 is inactivated.

### Immunogenic Fragments

The present invention provides an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids (e.g. less than 445 amino acids, less than 300 amino acids, less than 150 amino acids etc.)

In an embodiment the immunogenic fragment of the invention comprises an epitope within a consensus sequence of YNELQD-[A/Q]-YA-[QK/KQ]-QTE (SEQ ID NO: 64), e.g. SEQ ID NO: 60, 61, 62 or 63 and is less than 448, 440, 425, 415, 407, 400, 395, 380, 375, 260, 250, 240, 330, 320, 205, 300, 295, 290, 280, 270, 255, 250, 240, 230, 215, 200, 190, 180, 170, 160, 150, 140, 130, 120, 110, 100, 95, 90, 85, 80, 75, 70, 65, 69, 55, 50, 45, 40, 35, 30, 25, 24, 23, 22, 21, 20, 19, 18, 17, 16, 15 or 14 continuous amino acids in length. In an embodiment the immunogenic fragment of UspA2 of the invention is between 449 and 14 amino acids in length. In an embodiment, the immunogenic fragment of UspA2 of the invention comprising an epitope within a consensus sequence of YNELQD-[A/Q]-YA-[QK/KQ]-QTE (SEQ ID NO: 64), e.g. SEQ ID NO: 60, 61, 62 or 63, is between 14-449 amino acids, 100-449 amino acids, 125-449, 150-400 amino acids, 200-499 amino acids etc. In an embodiment, the epitope of the invention is within a consensus sequence of YNELQD-[A/Q]-YA-[QK/KQ]-QTE (SEQ ID NO: 64), e.g. SEQ ID NO: 60, 61, 62 or 63. In an embodiment the epitope of the invention is the consensus sequence of SEQ ID NO: 60, 61, 62 or 63.

In an embodiment the consensus sequence is YNELQD-[A/Q]-YA-[QK/KQ]-QTE. Amino acids shown in squared parenthesis (e.g. [A/Q]) correspond to variable regions where the amino acid of the consensus sequence may be either of the amino acid options provided. Variable amino acids are separated by the forward-slash (/) symbol. Taking the variable regions into account, the consensus sequence or epitope of the invention may be present in any of the following embodiments;

(SEQ ID NO: 60)
YNELQD-[A]-YA-[QK]-QTE
(SEQ ID NO: 61)
YNELQD-[A]-YA-[KQ]-QTE
(SEQ ID NO: 62)
YNELQD-[Q]-YA-[QK]-QTE
(SEQ ID NO: 63)
YNELQD-[Q]-YA-[KQ]-QTE

In an embodiment the consensus sequence corresponds to YNELQ-[X]z-QTE wherein X is an amino acid and Z is 2, 4, 6 or 8. In an embodiment Z is 6. In a further embodiment, the [X]z region may comprise up to two amino acid modifications. Said amino acid modifications may comprise substitution of glutamine (Q) with alanine (A) or vice versa and/or inversion of glutamine (Q) with lysine (K) or vice versa. In an embodiment the [X]z region may be DAYAKQ, DAYAQK, DQYAKQ, DQYAQK, DAYAKA, DAYAAK wherein the full-length epitope is SEQ ID NO: 60, 61, 62 or 63.

In an embodiment the immunogenic fragment of UspA2 of the invention (i.e. the immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) is taken from a sequence with at least 80% identity (e.g. at least 80%, at least 85%, at least 90%, at least 95%, at least 97% or at least 100% identity) to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

Any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75, or any sequence with at least 80% identity to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75 may be used as the base sequence from which the immunogenic fragment of the invention is derived from.

In an embodiment the immunogenic fragment of UspA2 of the invention (i.e. the immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) is taken from a sequence with at least 80% identity (e.g. at least 80%, at least 85%, at least 90%, at least 95%, at least 97% or at least 100% identity) to the entire length of SEQ ID NO: 1.

In an embodiment, the immunogenic fragment of UspA2 of the invention comprises an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 150 continuous amino acids (e.g. less than 145, 132, 125, 120, 110, 100, 90, 75, 60, 50, 40, 30 or 20 continuous amino acids). In an embodiment the immunogenic fragment of UspA2 of the invention comprises an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 150 continuous amino acids and wherein UspA2 has at least 80% identity to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment, the immunogenic fragment of UspA2 of the invention is at least 14 amino acids (e.g. greater than 14 amino acids, greater than 50 amino acids, greater than 100 amino acids, greater than 132 amino acids, greater than 200 amino acids, greater than 300 amino acids, greater than 400 amino acids) and comprises an epitope within a consensus sequence of YNELQD-[A/Q]-YA-[QK/KQ]-QTE (SEQ ID NO: 64), e.g. SEQ ID NO: 60, 61, 62 or 63. In an embodiment the immunogenic fragment of UspA2 of the invention comprises an epitope within a consensus sequence of YNELQD-[A/Q]-YA-[QK/KQ]-QTE (SEQ ID NO: 64), e.g. SEQ ID NO: 60, 61, 62 or 63, wherein said fragment is at least 14 amino acids and wherein UspA2 has at least 80% identity to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In an embodiment, the immunogenic fragment of the invention is less than 449 continuous amino acids but at least 14 continuous amino acids. In an embodiment, the immunogenic fragment of the invention is combined with further T-cell epitopes to promote immunogenicity.

In an embodiment the immunogenic fragment of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63 wherein said fragment is less than 449 amino acids) is capable of generating an immune response against M. catarrhalis. UspA2 may be the full length UspA2 from any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75 or a sequence with at least 80% identity to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In an embodiment, said immune response is functional.

In an embodiment the immunogenic fragment of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63 wherein said fragment is less than 449 amino acids) is capable of generating a functional immune response against heterologous strain(s) M. catarrhalis. UspA2 may be the full length UspA2 from any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75 or a sequence with at least 80% identity to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment the epitope of the immunogenic fragment of the invention (i.e. the epitope within a consensus sequence of YNELQD-[A/Q]-YA-[QK/KQ]-QTE (SEQ ID NO: 64), e.g. SEQ ID NO: 60, 61, 62 or 63,) confers cross-bactericidal activity against heterologous strain(s) of M. catarrhalis. In an embodiment, the present invention provides an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, which confers cross-bactericidal activity against heterologous strain(s) of M. catarrhalis wherein said fragment is less than 449 continuous amino acids and wherein UspA2 has at least 80% identity to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. Said epitope, when present within the immunogenic fragment of the invention, confers cross-bactericidal activity against 1 or more strain or strains of M. catarrhalis. As used herein, the term strain(s) refers to both strain (singular) and strains (plural). Cross-bactericidal activity can be measured by known techniques of the art which are described in detail in Example 4 (e.g. cross-bactericidal activity can be measured using the SBA assay).

In an embodiment the immunogenic fragment of UspA2 comprises an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids and is cross-protective against heterologous strain(s) of M. catarrhalis. UspA2 may be UspA2 from SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75 or a sequence with at least 80% identity to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. The cross-protectivity of the immunogenic fragment can be measured using studies as described in (23); the contents of which are incorporated herein by reference. The cross-protectivity of the immunogenic fragment of the invention relates to strain(s) of M. catarrhalis which are heterologous in comparison to SEQ ID NO:1. Alignments and sequence identities can be measured as described below (for example using Gap and Needle programs).

In an embodiment the immunogenic fragment of the invention provides an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids and wherein UspA2 has at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9% or 100%) identity to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

Alignments between polypeptides pairs may be calculated by various programs. For example, the Needle program from the EMBOSS package (24) and the Gap program from the GCG (a US registered trademark) package (Accelrys Inc.) may be used.

The Gap and Needle programs are an implementation of the Needleman-Wunsch algorithm described in (25). These programs are using frequently the BLOSUM62 scoring matrix (26) with gap open and extension penalties of, respectively, 8 and 2. Sometimes, the PAM250 scoring matrix (Dayhoft et al., (1978), “A model of evolutionary changes in proteins”, In “Atlas of Protein sequence and structure” 5(3) M. O. Dayhoft (ed.), 345-352, National Biomedical Research Foundation, Washington) is also used.

Scoring matrices are describing by numbers the tendency of each amino acid to mutate in another, or to be conserved. These numbers are generally computed from statistics of mutations observed in faithful pairwise or multiple alignments, or even in fragments of multiple alignments. Generally, in these tables, if a high positive number is associated with a pair of identical amino acids, it is indicating that this residue has a low tendency for mutation. At the opposite, a high positive number associated with a pair of different amino acids is indicating a high tendency of mutation between these two. And this is called a “conservative substitution”.

Looking at a pairwise alignment, aligned identical residues (“identities”) between the two sequences can be observed. A percentage of identity can be computed by multiplying by 100 (1) the quotient between the number of identities and the length of the alignment (for example, in the Needle program output), or (2) the quotient between the number of identities and the length of the longest sequence, or (3) the quotient between the number of identities and the length of the shortest sequence, or (4) the quotient between the number of identities and the number of aligned residues (for example, in the Gap program output).

Generally, sequence identity is calculated over the entire length of the reference sequence (i.e. SEQ ID NO: 1), for example the full-length or wild-type sequence. Amino acid substitution may be conservative or non-conservative. In some embodiments, amino acid substitution is conservative. Substitutions, deletions, additions or any combination thereof may be combined in a single variant so long as the variant is an immunogenic polypeptide. Embodiments herein relating to “vaccine compositions” of the invention are also applicable to embodiments relating to “immunogenic compositions” of the invention, and vice versa.

In an embodiment, the immunogenic fragment of the invention comprises an epitope within a consensus sequence of YNELQD-[A/Q]-YA-[QK/KQ]-QTE (SEQ ID NO: 64), e.g. SEQ ID NO: 60, 61, 62 or 63, wherein the consensus sequence is located within the stalk region of UspA2. UspA2 is a homotrimer which presents as a highly elongated and stable structure. UspA2 is composed of a N-terminal head region, followed by a stalk which ends by an amphipathic helix and a C-terminal membrane domain. The stalk region is composed of several canonical heptad repeats. In an embodiment the consensus sequence of the invention is located within the stalk region of UspA2. In an embodiment the epitope of the invention is located within a consensus sequence of the invention which is located in the stalk region of UspA2. In an embodiment the immunogenic fragment of the invention comprises an epitope within a consensus sequence of YNELQD-[A/Q]-YA-[QK/KQ]-QTE (SEQ ID NO: 64), e.g. SEQ ID NO: 60, 61, 62 or 63, wherein the consensus sequence is present at up to 15 locations within the stalk region of UspA2, e.g. 1, 2, 3, 4, 5, 6, 7, 8, 9, 10, 11, 12, 13, 14 or 15 times. For example, in relation to the reference strain of SEQ ID NO: 1 the consensus sequence of the invention is present in 3 locations (i.e. amino acids 308-321, amino acids 343-356 and amino acids 378-391). The consensus sequence of the invention is located within the LAAY-KASS repeat sequences of the stalk region. More specifically the consensus sequence of the invention is located within LAAY repeat region which is defined by the 22 amino acid sequence of SEQ ID NO: 80. UspA2 proteins have a variable number of LAAY-KASS sequences. The UspA2 proteins follow a trend very similar to that of UspA1 proteins which also contain highly conserved LAAY-KASS sequences. The conserved regions of UspA2 are reported in (4, 21)

### Antigen Binding Protein

According to a further aspect of the invention, there is provided an antigen binding protein which binds to an epitope within a consensus sequence of YNELQD-[A/Q]-YA-[QK/KQ]-QTE (SEQ ID NO: 64), e.g. SEQ ID NO: 60, 61, 62 or 63. The antigen binding protein of the invention specifically binds to UspA2 at the consensus sequence of the invention. As the epitope of the invention is within the consensus sequence of the invention the antigen binding protein also binds the epitope. The antigen binding protein of the invention binds to the consensus sequence and/or epitope of the invention at multiple sites (up to 15 locations within the Stalk region of UspA2). The antigen binding protein of the invention is also able to bind UspA1. The antigen binding protein of the invention can promote UspA2 intermolecular bridging and can bind secondary motifs and repeat regions.

In an embodiment the antigen binding protein of the invention is an antibody. In an embodiment the antibody is a mouse monoclonal antibody (mAB). In an embodiment the isotype of the mAB is a mouse IgG2A. In an embodiment, the mAB is FHUSPA2/10. In an embodiment the antibody of the invention is produced by the Repetitive Immunisation Multiple Sites (RIMMS) method which is described in (27) and is enclosed by reference.

In an embodiment, the antigen binding protein of the invention comprises (i) any one or a combination of CDRs selected from CDRH1, CDRH2, CDRH3 from SEQ ID NO: 82, and/or CDRL1, CDRL2, CDRL3 from SEQ ID NO: 84; or (ii) a CDR variant of (i), wherein the variant has 1, 2, or 3 amino acid modifications in each CDR.

In an embodiment, the antigen binding protein of the invention comprises any one or a combination or all of the following CDRs: (a) CDRH1 of SEQ ID NO: 85; (b) CDRH2 of SEQ ID NO: 86; (c) CDRH3 of SEQ ID NO: 87; (d) CDRL1 of SEQ ID NO: 88; (e) CDRL2 of SEQ ID NO: 89; and/or (f) CDRL3 of SEQ ID NO: 90. The CDRs were determined by Kabat. The antigen binding protein may comprise: a humanised VH region, or a humanised Heavy Chain (HC) sequence; and/or a humanised VL region, or a humanised Light Chain (LC) sequence, which comprise the CDRs as described above.

In an embodiment, the antibody of the invention comprises a Variable Heavy (VH) region comprising a sequence at least 80% identical (e.g. at least 85%, 90%, 91%, 92%, 93%, 94%, 95%, 96%, 97%, 98%, 99% or 100% identical) the sequence of SEQ ID NO: 82; and/or a Variable Light (VL) region comprising a sequence at least 80% identical (identical (e.g. at least 85%, 90%, 91%, 92%, 93%, 94%, 95%, 96%, 97%, 98%, 99% or 100% identical) to the sequence of SEQ ID NO: 84. In an embodiment the antibody of the invention comprises a Variable Heavy (VH) region encoded by sequence at least 80% identical (e.g. at least 85%, 90%, 91%, 92%, 93%, 94%, 95%, 96%, 97%, 98%, 99% or 100% identical) the sequence of SEQ ID NO: 81; and/or a Variable Light (VL) region encoded by a sequence at least 80% identical (identical (e.g. at least 85%, 90%, 91%, 92%, 93%, 94%, 95%, 96%, 97%, 98%, 99% or 100% identical) to the sequence of SEQ ID NO: 83.

The antigen binding protein sequence may be a variant sequence with up to 3 amino acid modifications. For example the modification is a substitution, addition or deletion. For example, the variant sequence may have up to 3, 2 or 1 amino acid substitution(s), addition(s) and/or deletion(s). The sequence variation may exclude the CDR(s), for example the CDR(s) is intact and the variation is in the remaining portion of the VH or VL (or HC or LC) sequence, so that the CDR sequence is fixed. The variant sequence substantially retains the biological characteristics of the unmodified antigen binding protein.

As used herein the term “VH Region” or “VL Region” refers to the variable portions of the heavy (VH) and light (VL) chains respectively. These regions form the binding pocket, which binds the specific antigens, and contains the major diversity of the immunoglobulin.

In an embodiment the antibody of the invention comprises a VH region comprising SEQ ID NO: 82; and/or a VL region comprising SEQ ID NO: 84. In an embodiment the antibody of the invention comprises a VH region encoded by the sequence of SEQ ID NO: 81; and/or a VL region encoded by the sequence of SEQ ID NO: 84.

In an embodiment the antigen binding protein of the invention is able to bind within the consensus sequence of SEQ ID NO: 64 and promote bactericidial activity. In an embodiment the antigen binding protein of the invention is able to bind to an epitope within SEQ ID NO: 60, 61, 62 or 63 and promote bactericidal activity.

The invention further provides an antigen binding protein that binds to UspA2, and competes for binding to the consensus sequence SEQ ID NO: 64 with a reference antibody with a VH region comprising SEQ ID NO: 82 and a VL region comprising SEQ ID NO: 84. Suitable assays to analyse whether antibodies compete for binding are well known in the art (for example see Kwak & Yoon et al 1996, J Immunol Methods 191(1): 49-54).

The binding of the antibody of the invention to a consensus sequence of YNELQD-[A/Q]-YA-[QK/KQ]-QTE (SEQ ID NO: 64) e.g. SEQ ID NO: 60, 61, 62 or 63, can be determined using Hydrogen-Deuterium exchange coupled with Mass Spectrometry (HDX-MS). Briefly, HDX-MS detects structural changes of a protein due to ligand binding, protein-protein interaction, post-translational modifications and others (the method is described in Example 3). The epitope region on the UspA2 which is targeted by mAB FHUSPA2/10 will display reduced exchange rates in the presence of FHUSPA2/10 relative to UspA2 alone which can be identified by HDX-MS. Following the exchange, the reaction is quenched with an acidic pH and low temperature. The proteins are digested with pepsin or other acidic proteases and analysed via mass spectrometry.

The present invention also provides a nucleic acid sequence which encodes the antigen binding protein as defined herein.

The present invention also provides an expression vector comprising the nucleic acid sequence as defined herein.

The present invention also provides a recombinant host cell comprising the nucleic acid sequence as defined herein, or the expression vector as defined herein.

The present invention also provides a method for the production of the antigen binding protein as defined herein, which method comprises culturing the host cell as defined herein under conditions suitable for expression of said nucleic acid sequence or vector, whereby the antigen binding protein is expressed and purified.

The present invention also provides an antigen binding protein produced by the method described herein.

The present invention also provides a pharmaceutical composition comprising the antigen binding protein as defined herein, and one or a combination of pharmaceutically acceptable carriers, excipients or diluents.

### Use of Immunogenic Fragments

According to a further aspect of the invention there is provided an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids for use in preventing or treating an infection, disease or condition caused by M. catarrhalis. In an embodiment, the immunogenic fragment of UspA2 of the invention, for use in preventing or treating an infection, disease or condition caused by M. catarrhalis, is taken from a sequence with at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment, there is provided an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids for use in preventing or treating an infection disease or condition caused by heterologous strain(s) of M. catarrhalis. In an embodiment, the immunogenic fragment of UspA2 of the invention, for use in preventing or treating an infection, disease or condition caused by heterologous strain(s) of M. catarrhalis, is taken from a sequence with at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment, there is provided an immunogenic fragment of UspA2 of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) for use in the prevention or treatment of an infection disease or condition caused by heterologous strain(s) of M. catarrhalis wherein said strain(s) comprise a surface protein having a sequence with 40%-99% identity (e.g. 40-50% identity, 45-65% identity, 50%-70% identity, 50-99% identity etc.) to SEQ ID NO:1. Said surface protein is optionally UspA2 or may be UspA2H or UspA2V. For example, the immunogenic fragment of the invention may be used to prevent or treat an infection, disease or condition caused by heterologous strain(s) of M. catarrhalis said heterologous strain(s) may have an UspA2 surface protein between 40%-99% identical to SEQ ID NO:1 (i.e. the strain(s) of M. catarrhalis being treated or protected against may have UspA2 sequences more than 40% identical, more than 50% identical, more than 60% identical, more than 70% identical, more than 75% identical, more than 80% identical more than 90% identical or more than 95% identical to SEQ ID NO:1).

Examples of such strains are shown in Example 4 but the invention is not so limited to these strains. Furthermore, the invention is not so limited to the prevention or treatment of an infection, disease or condition caused by heterologous strain(s) of M. catarrhalis wherein said strain(s) comprise a surface protein having at least 40-99% identity to SEQ ID NO: 1 because the invention further includes the use of the immunogenic fragment of the invention for the prevention or treatment of an infection, disease or condition caused by strain(s) which are 100% identical to SEQ ID NO:1.

In an embodiment, there is provided an immunogenic fragment of UspA2 of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) for use in preventing or treating an infection, disease or condition caused by heterologous strain(s) of M. catarrhalis comprising UspA2H, UspA2V or a variant thereof. In an embodiment said fragment is taken from a sequence with at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75

Studies with clinical isolates suggested that UspA1 is expressed by almost all isolates (99%) (28). The UspA1 protein of Moraxella catarrhalis has been shown to function as an adhesin that mediates adherence to human epithelial cell lines in vitro (29). UspA1 has been shown to bind carcinoembryonic antigen-related cellular adhesion molecule 1 (CEACAM1)(30, 31), fibronectin (5), laminin (6), and the serum complement factors C3 (32) and C4b binding protein (33).

The UspA2 gene shows three variants (e.g. UspA2, UspA2H and a rare UspA2V variant). In addition to UspA1, either UspA2 or UspA2H are expressed at a population ratio of approximately 4:1 (29, 34). M. catarrhalis strains can express either a UspA2 protein or an UspA2H protein or an UspA2V protein. The N-terminal head-neck domain and the C-terminal membrane spanning region between UspA1 and UspA2 are highly divergent, whereas their stalk region is more homologous (4, 21, 29). UspA2H is a considered a protein hybrid molecule with its N-terminal region being virtually identical to that of UspA1 proteins (More specifically, this region of the UspA2H protein contains the GGG amino acid repeats and the FAAG region, which have been observed in the N termini of all UspA1 proteins examined to date) whereas its C-terminal region is highly similar to that of the UspA2 protein. UspA2H proteins have been shown to function as adhesins likely because of the presence of the UspA1-like domains (Lafontaine 2000). Recently a new variant of UspA2 (UspA2V) containing UspA1 Carcinoembryonic antigen related cell adhesion molecule (CEACAM) binding domain at the C-terminal was reported in 14% of M. catarrhalis isolates thus contributing to cellular adherence (22).

In an embodiment the present invention provides an immunogenic fragment of the invention (i.e. immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) for use in preventing or treating an infection, disease or condition caused by strain(s) of M. catarrhalis which are UspA1*/UspA2-. In an embodiment said fragment is taken from a sequence with at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In a further embodiment said immunogenic fragment of the invention is used to protect or treat an infection, disease or condition caused by strains of M. catarrhalis which does not express UspA2 but expresses UspA2H. In a further embodiment the immunogenic fragment of the invention is used to protect or treat an infection, disease or condition caused by strains of M. catarrhalis which does not express UspA2 or UspA2H but expresses UspA2V. UspA1*/UspA2-strains correspond to strains of M. catarrhalis which are UspA1-positive/UspA2-negative i.e. strains which do not express UspA2 or a variant of UspA2 (i.e. UspA2 independent).

In an embodiment the immunogenic fragment of the invention (i.e. the immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) is used to prevent or treat an infection, disease or condition caused by heterologous strain(s) of M. catarrhalis for example otitis media, pneumonia and/or acute exacerbations of chronic obstructive pulmonary disease (AECOPD). In an embodiment, the immunogenic fragment of UspA2 for use in preventing or treating an infection, disease or condition caused by heterologous strain(s) of M. catarrhalis which otitis media, pneumonia and/or acute exacerbations of chronic obstructive pulmonary disease (AECOPD) is taken from a sequence at least 80% identical to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In an embodiment of the invention, such treatment or prevention comprises boosting a pre-existing immune response against M. catarrhalis by administering the immunogenic fragment of the invention to a subject (preferably a human subject) in an amount sufficient to elicit an immune response. More particularly to increase a pre-exciting immune response. The immunogenic fragment of the invention may also be used against any other infection, disease or condition caused by heterologous strain(s) of M. catarrhalis. The immunogenic fragment of the invention may be used to prevent or treat any subtype of AECOPD (e.g. type I, type 2 or type 3 exacerbations wherein further symptoms may include dyspnea, sputum volume, sputum purulence, coughing, wheezing or other symptoms of upper respiratory tract infection), otitis media (e.g. acute otitis media, chronic otitis media with/without effusion) or pneumonia. In an embodiment the immunogenic fragment of the invention (i.e. the immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) is used to prevent or treat acute exacerbations of chronic obstructive pulmonary disease (AECOPD).

### Cross-Bactericidal Activity

A further aspect of the invention provides an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 continuous amino acids (e.g. less than 500, less than 455, less than 400, less than 320, less than 270, less than 200, less than 125, less than 75, less than 50 continuous amino acids) for use in eliciting cross-bactericidal activity against heterologous strain(s) of M. catarrhalis. In an embodiment, the immunogenic fragment of UspA2 of the invention comprising an epitope within a consensus sequence of YNELQD-[A/Q]-YA-[QK/KQ]-QTE (SEQ ID NO: 64), e.g. SEQ ID NO: 60, 61, 62 or 63 for use in eliciting cross-bactericidal activity against heterologous strain(s) of M. catarrhalis is between 14-509, 14-500 amino acids, 100-499, 100-449 amino acids, 125-509, 150-475 amino acids, 150-400 amino acids, 200-499 amino acids etc.

In an embodiment the immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 amino acids, for use in eliciting cross-bactericidal activity against heterologous strain(s) of M. catarrhalis is taken from a sequence with at least 80% identity to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. UspA2 may be UspA2 from any sequence with at least 80% identity (e.g. at least 80%, at least 81%, at least 82%, at least 83%, at least 84%, at least 85%, at least 86%, at least 88%, at least 90%, at least 91%, at least 93%, at least 95%, at least 97%, at least 99%, at least 99.5%, at least 99.7%, at least 99.9% or 100% identity) to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In an embodiment said heterologous strain(s) of M. catarrhalis are strains which differ from the strain used to immunise a subject. In an embodiment, said strain(s) have diverse sequences in the range of 40%-99% (e.g. 40-95%, 45-85%, 45-80%, 50-90%, 55-85%, 60-99%, 63-95% identity to SEQ ID NO:1.) In an embodiment the epitope of the immunogenic fragment (i.e. the epitope within a consensus sequence of YNELQD-[A/Q]-YA-[QK/KQ]-QTE (SEQ ID NO: 64), e.g. SEQ ID NO: 60, 61, 62 or 63,) is responsible for conferring the cross-bactericidal activity of the fragment as a whole.

Cross-bactericidal activity is measured using an in vitro serum bactericidal assay (SBA) as described in Example 4. The SBA measures functional M. catarrhalis specific antibodies capable of complement mediated bacterial killing and is widely recognised in the art as a surrogate assay for the evaluation of immunogenicity of bacterial vaccines due to its close correlation with protection.

The present invention further provides an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids for use in eliciting cross-bactericidal activity against heterologous strain(s) of M. catarrhalis.

In an embodiment an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 continuous amino is used to elicit cross-bactericidal activity against heterologous strain(s) of M. catarrhalis wherein said strain(s) comprise UspA2H, UspA2V or a variant thereof. In a separate embodiment cross-bactericidal activity is conferred only in the presence of either UspA2 or UspA2H. In this embodiment, UspA2 may be any sequence with at least 80% identity to any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment said fragment of UspA2 (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 continuous amino acids) is used to elicit cross-bactericidal activity against heterologous strain(s) of M. catarrhalis wherein said strain(s) cause otitis media, pneumonia and/or acute exacerbations of chronic obstructive pulmonary disease (AECOPD). In this embodiment, UspA2 may be any sequence with at least 80% identity to any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

### Immunogenic Compositions

The present invention provides an immunogenic composition comprising the immunogenic fragment of the invention. In an embodiment the immunogenic composition comprises an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids (e.g. less than 448, 440, 425, 415, 407, 400, 395, 380, 375, 260, 250, 240, 330, 320, 205, 300, 295, 290, 280, 270, 255, 250, 240, 230, 215, 200, 190, 180, 170, 160, 150, 140, 130, 120, 110, 100, 95, 90, 85, 80, 75, 70, 65, 69, 55, 50, 45, 40, 35, 30, 25, 24, 23, 22, 21, 20 continuous amino acids). In an embodiment, said fragment is taken from an UspA2 sequence with at least 80% identity (e.g. at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8% 99.9% or 100% identity) to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment the immunogenic composition comprises an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 150 continuous amino acids (e.g. less than 150, 140, 130, 120, 110, 100, 95, 90, 85, 80, 75, 70, 65, 69, 55, 50, 45, 40, 35, 30, 25, 24, 23, 22, 21, 20, 19, 18, 17, 16, 15 or 14 continuous amino acids). In an embodiment the immunogenic composition comprises the immunogenic fragment of UspA2 of the invention wherein said fragment is at least 14 amino acids in length and comprises the epitope within a consensus sequence of YNELQD-[A/Q]-YA-[QK/KQ]-QTE (SEQ ID NO: 64), e.g. SEQ ID NO: 60, 61, 62 or 63. In both of these embodiments, UspA2 may be any sequence with at least 80% identity to any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment the immunogenic composition of the invention comprises an immunogenic fragment of UspA2 wherein the epitope (i.e. the epitope within a consensus sequence of YNELQD-[A/Q]-YA-[QK/KQ]-QTE (SEQ ID NO: 64), e.g. SEQ ID NO: 60, 61, 62 or 63,) confers cross-bactericidal activity against heterologous strain(s) of M. catarrhalis. In an embodiment the immunogenic composition of the invention comprises an immunogenic fragment of UspA2 wherein the epitope (i.e. the epitope within a consensus sequence of YNELQD-[A/Q]-YA-[QK/KQ]-QTE (SEQ ID NO: 64), e.g. SEQ ID NO: 60, 61, 62 or 63,) is cross-protective against heterologous strain(s) of M. catarrhalis.

In an embodiment the immunogenic composition of the invention comprises an immunogenic fragment comprising or consisting of a consensus sequence which is located within the stalk region of UspA2. In an embodiment the immunogenic composition of the invention comprises an immunogenic fragment comprising or consisting of consensus sequence which is present at up to 15 locations within the stalk region of UspA2.

In an embodiment the immunogenic composition comprises the immunogenic fragment of the invention as a fusion protein.

In an embodiment the immunogenic composition of the invention may be administered with other antigens. For example, the present immunogenic composition may be administered with antigens from H. influenzae. In an embodiment the immunogenic composition of the invention comprises the immunogenic fragment of the invention (i.e. immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63 wherein said fragment is less than 449 continuous amino acids) and at least one antigen (e.g. one or more antigens) from Haemophilus influenzae. For example, in an embodiment the immunogenic fragment of the invention may be administered with Protein D (PD) from H. influenzae, Protein E (PE) from H. influenzae and/or Pillin A (PiA) from H. influenzae.

### Protein D (PD)

The immunogenic composition for use in the invention may comprise protein D or an immunogenic fragment thereof from Haemophilus influenzae. Protein D (PD) is a highly conserved 42 kDa surface lipoprotein found in all Haemophilus influenzae, including nontypeable Haemophilus influenzae. Inclusion of this protein in the immunogenic composition may provide a level of protection against Haemophilus influenzae related otitis media (12). Suitable amino acid sequences for PD include, for example, the protein D sequence from FIG. 9 of (35)(FIGS. 9a and 9b together, 364 amino acids) and as described in (36) or (37) (disclosed herein as SEQ ID NO: 39 and 40). Other suitable proteins may be encoded by, for example, Genbank accession numbers: X90493 (SEQ ID NO:41), X90489 (SEQ ID NO:42), X90491 (SEQ ID NO:43), Z35656 (SEQ ID NO:44), Z35657 (SEQ ID NO:45), Z35658 (SEQ ID NO:46), M37487 (SEQ ID NO:47). Protein D may be used as a full-length protein or as a fragment. For example, a protein D sequence may comprise (or consist) of the protein D fragment described in (35) which begins at amino acid 20 of SEQ ID NO: 39 (i.e. the sequence SSHSSNMANT (SerSerHisSerSerAsnMetAlaAsnThr) (SEQ ID NO. 67), and lacks the 19 N-terminal amino acids from SEQ ID NO: 39, optionally with the tripeptide MDP from NS1 fused to the N-terminal of said protein D fragment (348 amino acids) (SEQ ID NO: 40). In one aspect, the protein D or fragment of protein D is unlipidated.

One skilled in the art will further recognise that immunogenic compositions may comprise polypeptides having sequence identity to Protein D provided that such polypeptides are capable of generating an immune response to Protein D, for example, they comprise one or more epitopes of protein D. Thus, immunogenic compositions may comprise an isolated immunogenic polypeptide having sequence identity of at least 70%, 80%, 85%, 90%, 91%, 92%, 93%, 94%, 95%, 96%, 97%, 98%, 99%, or 100% to SEQ ID NO:39 wherein the isolated immunogenic polypeptide is capable of eliciting an immune response against SEQ ID NO:39, particularly an immune response that results in the formation of antibodies that bind to SEQ ID NO:39.

### Protein E (PE)

Protein E is an outer membrane lipoprotein with adhesive properties. It plays a role in the adhesion/invasion of non-typeable Haemophilus influenzae (NTHi) to epithelial cells (38-40). It is highly conserved in both encapsulated Haemophilus influenzae and non-typeable Haemophilus influenzae and has a conserved epithelial binding domain (41). Thirteen different point mutations have been described in different Haemophilus species when compared with Haemophilus influenzae Rd as a reference strain. Its expression is observed on both logarithmic growing and stationary phase bacteria (42).

Protein E is also involved in human complement resistance through binding vitronectin (38). PE, by the binding domain PKRYARSVRQ YKILNCANYH LTQVR (SEQ ID NO:48, corresponding to amino acids 84-108 of SEQ ID NO:49), binds vitronectin which is an important inhibitor of the terminal complement pathway (38).

Protein E from H. influenzae (also referred to as: “protein E”, “Prot E” and “PE”) may consist of or comprise the amino acid sequence of SEQ ID NO:49 (corresponding to SEQ ID NO:4 of (43). One skilled in the art will further recognise that immunogenic compositions may comprise polypeptides having sequence identity to Protein E provided that such polypeptides are capable of generating an immune response to Protein E, for example, they comprise one or more epitopes of Protein E. Thus, immunogenic compositions may comprise an isolated immunogenic polypeptide having sequence identity of at least 70%, 80%, 85%, 90%, 91%, 92%, 93%, 94%, 95%, 96%, 97%, 98%, 99%, or 100% to SEQ ID NO:49 wherein the isolated immunogenic polypeptide is capable of eliciting an immune response against SEQ ID NO:49, particularly an immune response that results in the formation of antibodies that bind to SEQ ID NO:49. The immunogenicity of PE polypeptides may be measured as described in (43) herein incorporated by reference.

In another aspect of the invention, the immunogenic composition comprises an immunogenic fragment of Protein E, suitably an isolated immunogenic polypeptide with at least 70%, 80%, 85%, 90%, 91%, 92%, 93%, 94%, 95%, 96%, 97%, 98%, 99%, or 100% identity to SEQ ID NO: 65 (corresponding to Seq ID No. 125 of reference 43).

### Pilin A (PiIA)

Pilin A (PiIA) is likely the major pilin subunit of H. influenzae Type IV Pilus (Tfp) involved in twitching motility (44). NTHi PiA is a conserved adhesin expressed in vivo. It has been shown to be involved in NTHi adherence, colonization and biofilm formation [26]. PiA may consist of or comprise the protein sequence of SEQ ID NO:50 (corresponding to SEQ ID NO. 58 of (43)). One skilled in the art will further recognise that immunogenic compositions may comprise polypeptides having sequence identity to Pilin A provided that such polypeptides are capable of generating an immune response to PiA, for example, they comprise one or more epitopes of PiA. Thus, immunogenic compositions may comprise an isolated immunogenic polypeptide having sequence identity of at least 70%, 80%, 85%, 90%, 91%, 92%, 93%, 94%, 95%, 96%, 97%, 98%, 99%, or 100% to SEQ ID NO:50 wherein the isolated immunogenic polypeptide is capable of eliciting an immune response against SEQ ID NO:50, particularly an immune response that results in the formation of antibodies that bind to SEQ ID NO:50. The immunogenicity of PiA polypeptides may be measured as described in (43) herein incorporated by reference.

In another aspect of the invention, the immunogenic composition comprises an immunogenic fragment of PiIA, suitably an isolated immunogenic polypeptide with at least 70%, 80%, 85%, 90%, 91%, 92%, 93%, 94%, 95%, 96%, 97%, 98%, 99%, or 100% identity to SEQ ID NO: 66 (corresponding to SEQ ID NO: 127 of reference 43).

In an embodiment the immunogenic composition of the invention may comprise the immunogenic fragment of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) and two antigens from H. influenzae optionally wherein the two antigens of H. influenzae are present as a fusion protein. In an embodiment the immunogenic composition of the invention comprises the immunogenic fragment of the invention and Protein E and Pilin A from H. influenzae. In an embodiment the immunogenic composition of the invention comprises the immunogenic fragment of the invention and Protein E and PiA in the form of a fusion protein (PE-PiIA). Suitable fusions are disclosed in (43)(enclosed here by reference) and a preferred fusion is LVL-735 of SEQ ID NO:51 (corresponding to sequence number 194 of (43)). Thus, the immunogenic composition may comprise a polypeptide having at least 70%, 80%, 85%, 90%, 91%, 92%, 93%, 94%, 95%, 96%, 97%, 98%, 99%, or 100% sequence identity to SEQ ID NO: 51 and/or 52 (PE-PiIA fusion without signal peptide). In any of these embodiments the immunogenic fragment of UspA2 may be a fragment taken from any sequence with at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

Thus, in particular embodiments of the invention, the immunogenic composition comprises both Protein E and PiA in the form of a fusion protein, suitably an isolated immunogenic polypeptide with at least 70%, 80%, 85%, 90%, 91%, 92%, 93%, 94%, 95%, 96%, 97%, 98%, 99%, or 100% to LVL-735, wherein the signal peptide has been removed, SEQ ID NO. 52 (Corresponding to SEQ ID No. 219 of (43)).

Thus, in particular embodiments of the invention, the immunogenic composition comprises both Protein E and PiA in the form of a fusion protein, suitably an isolated immunogenic polypeptide with at least 70%, 80%, 85%, 90%, 91%, 92%, 93%, 94%, 95%, 96%, 97%, 98%, 99%, or 100% to LVL-735, with the signal peptide, SEQ ID NO. 51 (Corresponding to SEQ ID No. 194 of (43)).

The immunogenicity of Protein E (PE) and Pilin A (PiIA) polypeptides may be measured as described in (43); the contents of which are incorporated herein by reference.

The amount of the immunogenic composition which is required to achieve the desired therapeutic or biological effect will depend on a number of factors such as the use for which it is intended, the means of administration, the recipient and the type and severity of the condition being treated, and will be ultimately at the discretion of the attendant physician or veterinarian. In general, a typical dose for the treatment of a condition caused in whole or in part by M. catarrhalis in a human, for instance, may be expected to lie in the range of from about 0.001 mg-0.120 mg. More specifically, a typical dose for the treatment of a condition caused wholly or in part by M. catarrhalis in a human may lie in the range of from about 0.003 mg to about 0.03 mg of protein. The present invention provides an immunogenic composition comprising the immunogenic fragment of the invention (i.e. immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) for use in the treatment or prevention of a condition or disease caused wholly or in part by M. catarrhalis. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. The immunogenic composition may contain additional antigens; a typical dose for the treatment of a condition caused wholly or in part by H. influenzae in a human may lie in the range of from about 0.005 mg to about 0.05 mg for each additional antigen. This dose may be administered as a single unit dose. Several separate unit doses may also be administered. For example, separate unit doses may be administered as separate priming doses within the first year of life or as separate booster doses given at regular intervals (for example, every 1, 5 or 10 years). The present invention also provides an immunogenic composition comprising the immunogenic fragment of the invention or a for use in the treatment or prevention of a condition or disease caused wholly or in part by Moraxella catarrhalis in combination with at least one antigen from Haemophilus influenzae.

Particular immunogenic compositions for use in the present invention will comprise (1) protein D, (2) a PE-PiIA fusion protein and (3) UspA2. In certain embodiments, the immunogenic composition for use in the present invention comprises an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids, a recombinant Protein D protein having at least 95% sequence identity to SEQ ID NO:39 and a recombinant PE-PiIA fusion protein having at least 95% sequence identity to SEQ ID NO: 51. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. Immunogenic compositions for use in the present invention may comprise (1) 10 μg of PD, (2) 10 μg of a PE-PiA fusion protein, (3) 10 μg of UspA2 and an (4) adjuvant, particularly AS01E. Immunogenic compositions for use in the present invention may comprise (1) 10 μg of PD, (2) 10 μg of a PE-PiIA fusion protein, (3) 3.3 μg of UspA2 and an (4) adjuvant, particularly AS01E. Particularly, the PE-PiIA fusion protein is the LVL735 construct (SEQ ID NO:51), as described in (43).

### Vaccines

The present invention provides a vaccine comprising the immunogenic fragment and/or immunogenic composition of the invention. In an embodiment the vaccine comprises an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids and compositions thereof. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75. In an embodiment the vaccine comprises the immunogenic fragment of the invention and at least one antigen from H. influenzae. In an embodiment the vaccine comprises the immunogenic fragment of UspA2 of the invention, Protein D or an immunogenic fragment thereof and/or PE-PiIA (e.g. LVL-735). In an embodiment the vaccine comprises the immunogenic fragment of the invention, at least one antigen from H. influenzae and an adjuvant (e.g. AS01E). Immunogenic compositions of the invention will generally comprise one or more adjuvants.

Suitable adjuvants include an aluminium salt such as aluminium hydroxide gel or aluminium phosphate or alum, but may also be a salt of calcium, magnesium, iron or zinc, or may be an insoluble suspension of acylated tyrosine, or acylated sugars, cationically or anionically derivatized saccharides, or polyphosphazenes. In one embodiment, the protein may be adsorbed onto aluminium phosphate. In another embodiment, the protein may be adsorbed onto aluminium hydroxide. In a third embodiment, alum may be used as an adjuvant.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see (45)); and a combination of monophosphoryl lipid A, preferably 3-de-O-acylated monophosphoryl lipid A, together with either an aluminium salt (for instance aluminium phosphate or aluminium hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (46) (47).

AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). AS01E is an Adjuvant System containing MPL, QS21 and liposomes (25 μg MPL and 25 μg QS21). In one embodiment, the immunogenic composition or vaccine of the invention comprises AS01. In another embodiment, the immunogenic composition or vaccine of the invention comprises AS01B or AS01E. In an embodiment, the immunogenic composition or vaccine comprises AS01E.

AS02 is an Adjuvant System containing MPL and QS21 in an oil/water emulsion. AS02V is an Adjuvant System containing MPL and QS21 in an oil/water emulsion (50 μg MPL and 50 μg QS21).

AS03 is an Adjuvant System containing α-Tocopherol and squalene in an oil/water (o/w) emulsion. AS03A is an Adjuvant System containing α-Tocopherol and squalene in an o/w emulsion (11.86 mg tocopherol). AS03B is an Adjuvant System containing α-Tocopherol and squalene in an o/w emulsion (5.93 mg tocopherol). AS03C is an Adjuvant System containing α-Tocopherol and squalene in an o/w emulsion (2.97 mg tocopherol). In one embodiment, the immunogenic composition or vaccine comprises AS03.

AS04 is an Adjuvant System containing MPL (50 μg MPL) adsorbed on an aluminium salt (500 μg A13+). In one embodiment, the immunogenic composition or vaccine comprises AS04.

A system involving the use of QS21 and 3D-MPL is disclosed in (48). A composition wherein the QS21 is quenched with cholesterol is disclosed in (49). An additional adjuvant formulation involving QS21, 3D-MPL and tocopherol in an oil in water emulsion is described in (50). In one embodiment the immunogenic composition additionally comprises a saponin, which may be QS21. The formulation may also comprise an oil in water emulsion and tocopherol (50). Unmethylated CpG containing oligonucleotides (51)) and other immunomodulatory oligonucleotides ((52) and (53)) are also preferential inducers of a TH1 response and are suitable for use in the present invention.

Additional adjuvants are those selected from the group of metal salts, oil in water emulsions, Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof.

Possible excipients include arginine, pluronic acid and/or polysorbate. In a preferred embodiment, polysorbate 80 (for example, TWEEN (a US registered trademark) 80) is used. In a further embodiment, a final concentration of about 0.03% to about 0.06% is used. Specifically, a final concentration of about 0.03%, 0.04%, 0.05% or 0.06% polysorbate 80 (w/v) may be used.

Formulations comprising the immunogenic compositions of the invention may be adapted for administration by an appropriate route, for example, by the intramuscular, sublingual, transcutaneous, intradermal or intranasal route. Such formulations may be prepared by any method known in the art. The invention further provides kits for use in the methods of the invention comprising a first container comprising a lyophilised immunogenic composition comprising (i) protein D from Haemophilus influenzae (PD) or a fragment thereof, (ii) Protein E from Haemophilus influenzae (PE) or a fragment thereof, (iii) pilin A from Haemophilus influenzae (PiIA) or a fragment thereof and (iv) Ubiquitous surface protein A2 from Moraxella catarrhalis (UspA2) or a fragment thereof and a second container comprising a liquid comprising AS01E. In certain particular embodiments, the kit further comprises a buffer. In certain other embodiments, the kit further comprises instructions for use.

### Methods of Treatment

In an embodiment the present invention provides a method of treatment or prevention of an infection, disease or condition caused by M. catarrhalis, in a subject in need thereof comprising administering to said subject a therapeutically effective amount of an immunogenic composition comprising the immunogenic fragment of the invention (e.g. an immunogenic composition comprising an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) or a vaccine comprising both the immunogenic fragment and immunogenic composition of the invention. In an embodiment the immunogenic fragment of the invention is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In an embodiment the present invention provides a method of treatment or prevention of acute exacerbations of chronic obstructive pulmonary disease (AECOPD), pneumonia and/or otitis media in a subject in need thereof comprising administering to said subject a therapeutically effective amount of an immunogenic composition comprising the immunogenic fragment of the invention (e.g. an immunogenic composition comprising an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) or a vaccine comprising both the immunogenic fragment and immunogenic composition of the invention. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75

The present invention provides use of (a) immunogenic fragment of the invention (i.e. immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids, (b) an immunogenic composition comprising the immunogenic fragment of the invention or (c) a vaccine comprising both the immunogenic fragment and composition of the invention for use in the manufacture of a medicament for the treatment or prevention of an infection, disease or condition caused by M. catarrhalis. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75

The present invention provides use of, (a) the immunogenic fragment of the invention (i.e. an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids), (b) an immunogenic composition comprising the immunogenic fragment of the invention or (c) a vaccine comprising both the immunogenic fragment and composition of the invention, for the manufacture of a medicament for treating or preventing acute exacerbations of pneumonia, otitis media and/or chronic obstructive pulmonary disease (AECOPD). In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

### Otitis Media

Otitis media is a major cause of morbidity in 80% of all children less than 3 years of age (54). More than 90% of children develop otitis media before age 7. In 2000, there were 16 million visits made to office-based physicians for otitis media in the United States and approximately 13 million antibacterial prescriptions dispensed (55). In European countries, the reported acute otitis media rates range between 0.125 to 1.24 per child-year (56). Otitis media is a costly infection and the most common reason children receive antibiotics (57). Bacteria are responsible for approximately 70% of cases of acute otitis media, with Streptococcus pneumoniae, non-typeable Haemophilus influenzae (NTHi), and Moraxella catarrhalis predominating as the causative agents (54). A subset of children experience recurrent and chronic otitis media and these otitis prone children have protracted middle-ear effusions that are associated with hearing loss and delays in speech and language development.). Recent antibiotic pressure and vaccination with the pneumococcal conjugate vaccine have resulted in the emergence of β-lactamase-producing Haemophilus influenzae and Moraxella catarrhalis as the leading organisms causing acute otitis media in North America, followed by Streptococcus pneumoniae (58).

Since otitis media is a multifactorial disease, the feasibility of preventing otitis media using a vaccination strategy has been questioned (57). The chinchilla model is a robust and validated animal model of otitis media and its prevention. While the chinchilla model may mimic the natural course of human infection, others have suggested that results in the chinchilla model may vary from one laboratory to the next. Various other rodents have also been used for the induction of otitis media and are summarized in (59). The murine animal model is often studied in otitis media research.

The presence of bactericidal antibody is associated with protection from otitis media due to non-typeable H. influenzae (60). However, an immune response need not be bactericidal to be effective against NTHi. Antibodies that merely react with NTHi surface adhesins can reduce or eliminate otitis media in the chinchilla.

Thus, in an embodiment the present invention provides a method of treatment or prevention of otitis media in a subject in need thereof comprising administering to said subject a therapeutically effective amount of an immunogenic composition comprising the immunogenic fragment of the invention (e.g. an immunogenic composition comprising an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) or a vaccine comprising both the immunogenic fragment and immunogenic composition of the invention. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

### AECOPD

Chronic Obstructive Pulmonary Disease (COPD), a common preventable disease, is characterised by persistent airflow limitation that is usually progressive. The airflow limitation is associated with an enhanced chronic inflammatory response in the airways and lungs to noxious particles of gases. The most important environmental risk factor for COPD is tobacco smoking, even though other factors, such as occupational exposure, may also contribute to the development of the disease. It is a multi-component disease that manifests as an accelerated decline in lung function, with symptoms such as breathlessness on physical exertion, deteriorating health status and exacerbations.

The prevalence of COPD is increasing: worldwide, COPD (GOLD grade II and above) affects 10.1±4.8% of the population >40 years of age (61). COPD is most prevalent in adults/elderly with a history of smoking (62). It is the fourth leading cause of chronic morbidity and mortality in the United States and the first in terms of disease burden in China. Recent papers report that in 2015, COPD ranked third among the global age-standardised death rates for both sexes, with about 3-2 million patients dying of the disease (63).

Acute exacerbations and comorbidities contribute to the overall disease severity in individual COPD patients. An acute exacerbation of COPD (AECOPD) is an acute event characterised by a worsening of the patient's respiratory symptoms that is beyond normal day-to-day variations and leads to a change in medication. AECOPD increases morbidity and mortality, leading to faster decline in lung function, poorer functional status (10).

The lungs are known to be colonised with different strains of bacteria (11, 64). In COPD patients, acquisition of new bacterial strains is believed to be an important cause of AECOPD (13). Although estimates vary widely, Non-Typeable Haemophilus influenzae (NTHi) appears to be the main bacterial pathogen associated with AECOPD (11-38%), followed by Moraxella catarrhalis (3-25%) and Streptococcus pneumoniae (4-9%) (14-16). Thus, in an embodiment the present invention provides a method of treatment or prevention of AECOPD in a subject in need thereof comprising administering to said subject a therapeutically effective amount of an immunogenic composition comprising the immunogenic fragment of the invention (e.g. an immunogenic composition comprising an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) or a vaccine comprising both the immunogenic fragment and immunogenic composition of the invention. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

### Pneumonia

Community-acquired pneumonia (CAP) has been described as the leading cause of death from infectious disease and the six-ranked cause of death overall in the United States. Moraxella catarrhalis is one of the pathogens associated with CAP in North America (65) and is one of the pathogens associated with moderate to severe community acquired pneumonia in Japan (66). Moraxella catarrhalis may be especially likely to cause pneumonia, endocarditis, septicaemia and meningitis in immunocompromised subjects. As well as being a primary causative, in some instance pneumonia in both adults and children can be exacerbated by Moraxella catarrhalis infection and pneumonia in children can be complicated by bacteraemia (presence of bacteria in blood) following bacterial infection of Moraxella catarrhalis.

Thus, in an embodiment the present invention provides a method of treatment or prevention of pneumonia in a subject in need thereof comprising administering to said subject a therapeutically effective amount of an immunogenic composition comprising the immunogenic fragment of the invention (e.g. an immunogenic composition comprising an immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids) or a vaccine comprising both the immunogenic fragment and immunogenic composition of the invention. In an embodiment, said immunogenic fragment is a fragment of UspA2 wherein UspA2 has at least 80% identity to the entire length of any of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

In order that this invention may be better understood, the following examples are set forth. These examples are for purposes of illustration only, and are not to be construed as limiting the scope of the invention in any manner.

### EXAMPLES

### Example 1: Computer Modeling of UspA2

### Background

Alpha helical trimeric coiled coil is a wide-spread protein architecture where three alpha helices are arranged around each other forming a supercoiled structure with a hydrophobic core. The packing of the side-chains in the core satisfies simple rules. In their simplest form, parallel homo-oligomers with a seven-residue sequence repeat pattern, the heptad repeat. Therein, the seven individual positions are labelled a, b, c, d, e, f, g, of which positions a and d correspond to hydrophobic residues. In addition to classical heptad repeats, related sequence periodicities can also be observed as eleven-residue hendecad repeats or heptdad repeats where hydrophilic residues (primarily N, but also H) can be found in position d or in both core positions, in motifs like such as SEQ ID NO: 76, SEQ ID NO: 77, SEQ ID NO: 78 and SEQ ID NO: 79 (ref 67).

Both UspA1 and UspA2 belong to the trimeric coiled coil adhesin protein family, characterized by a long tract of helix coiled coil in the extracellular domains (1). This implies that, in absence of an x-ray structure of UspA2, the identification of coiled coil repeats along the UspA2 sequence allows nevertheless to build a molecular model in silico with a high confidence degree.

### Materials and Methods

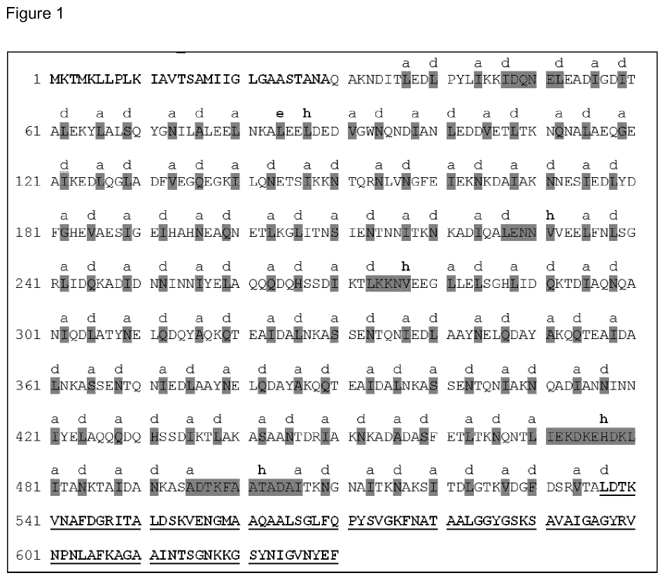

The amino acid sequence of UspA2 str. 25238 has been analysed to identify coiled coil periodicities. Such analysis predicts that the 37-537 region (numbering according to SEQ ID NO: 1) originates an uninterrupted helical trimeric coiled coil (FIG. 1).

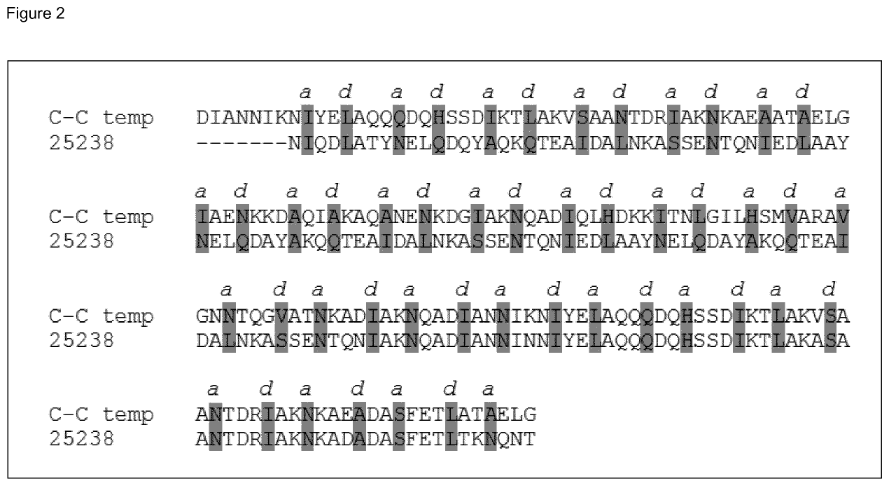

To build a computer model of the UspA2 ATCC25238 fragment 301-471 (numbering according to SEQ ID NO: 1), a trimeric coiled coil template has been generated using the Swiss-PDB software starting from the atomic coordinates of the 527-665 fragment of UspA1 solved by x-ray crystallography and publicly available at the PDB databank (PDB code 2QIH). UspA1 fragment 608-652 was fused to the N-terminal of 532-665 fragment to generate a final trimeric coiled coil 176 amino acid long. The 301-471 region of UspA2 25238 (numbering according to SEQ ID NO: 1) was then modelled superimposing the amino acid sequence onto the template, according to the alignment shown in FIG. 2.

### Results

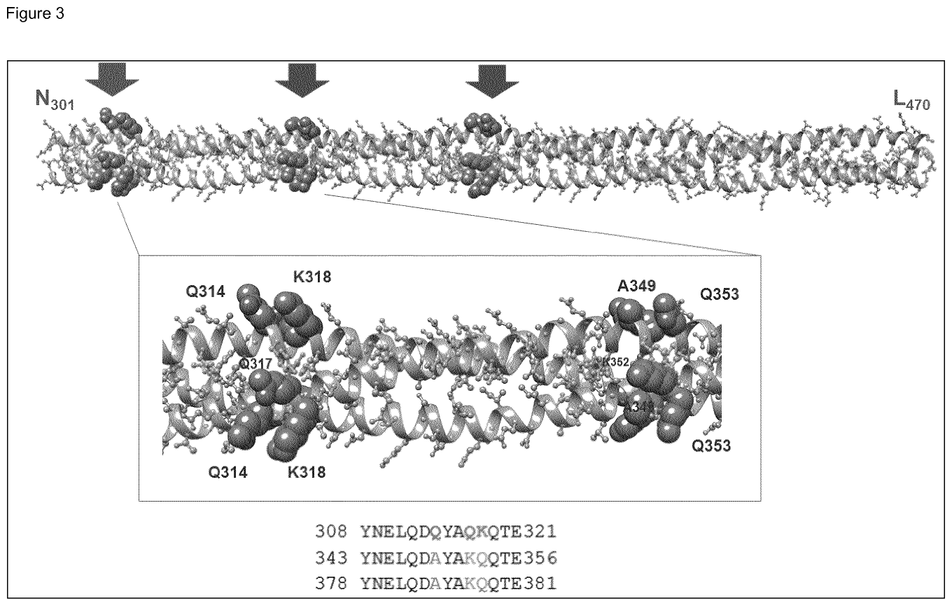

The computer model of the UspA2 region 301-471 is shown in FIG. 3. It is apparent that side chains of Q314, Q317 and K318 result well exposed to the external environment. Such amino acids are specifically present in the peptide 308-321 rendering this sequence unique as compared to the very similar peptides 343-356 and 378-381.

This observation implies despite the presence of a high degree of sequence similarity among 308-321, 343-356 and 378-381 peptides (numbering according to SEQ ID NO: 1), the first one constitutes a specific conformational epitope, which FHUSPA2/10 is able to discriminate.

### Example 2: Transmission Electron Microscopy

UspA2 (SEQ ID NO: 53) complex with FHUSPA2/10 mAb at different molar ratios.

The complex of the recombinant UspA2 (SEQ ID NO: 53) and the antibody FHUSPA2/10 was formed incubating the mAbs in molar ratios of 1:1, 1:3 and 1:6 for 1 hour at RT. The complexes were then purified using a Superose 6 3.2/30 (GE Healthcare) in TBS (25 mM Tris, 150 mM NaCl, pH 8). Additional attempts were performed using a HPLC equipped with Yarra3000 andYarra2000 (Phenomenex) in a mobile phase of 50 mM HEPES, 150 mM NaCl, pH 7. Fractions of 0.05 μl were collected by Fraction Collector Frac-950 (GE Healthcare).

The complexes were analysed by Transmission Electron Microscopy (TEM) using negative staining to verify the integrity of the sample and the formation of the complexes. The images of the recombinant protein alone were than processed to generate 3D structure with the aim to assess both the overall complex folding. Each sample was purified through a SEC (see complex formation and purification). The fraction of the chromatographic peak corresponding to apparent molecular weight of the immune complex was diluted to 0.03 mg/ml in 20 mM Tris, 300 mM NaCl, pH 8 buffer. A volume of 2.5 μl was loaded for 30 seconds onto a copper commercial 300-square mesh grid of carbon/formvar (Agar Scientific) previously glow discharged with 15 mA for 20 seconds using a Glow Discharge Quorum Q150AS.

Blotted off the excess of the solution by Whitman@filter Paper No. 1 (SIGMA-Aldrich), the grid was negatively stained using 1% of Phosphor Tungstic Acid (PTA) in water solution for 45 seconds. The excess of the stain was soaked off by Whitman filter paper No. 1. The images of the of the recombinant UspA2 alone or in complex with the mAb were collected on a Tecnai G2 Spirit TEM working at 120 kV with a side mount Olympus Morada 2K×4K CCD camera and a pixel size of 3.8 Å/pixel.

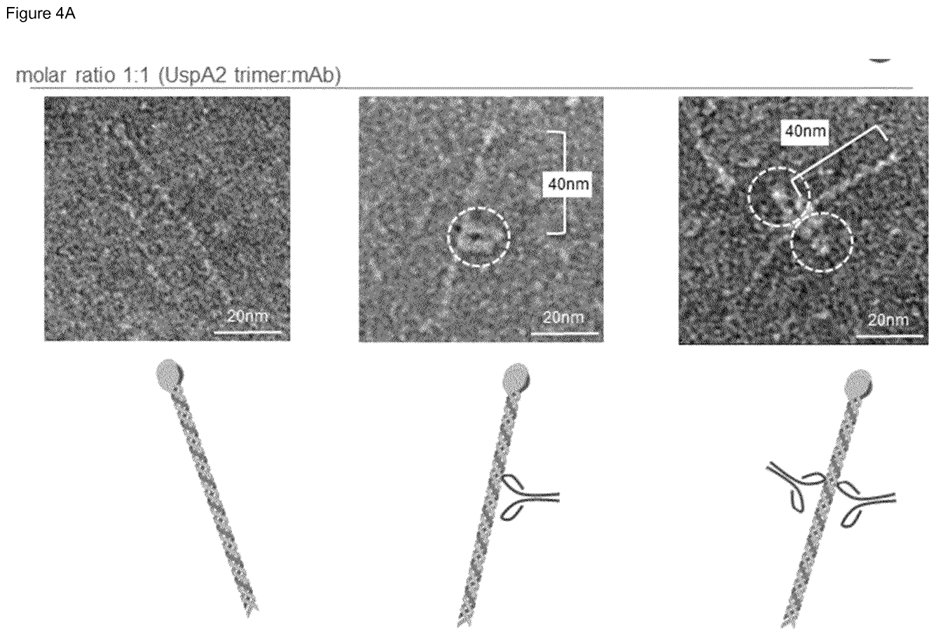

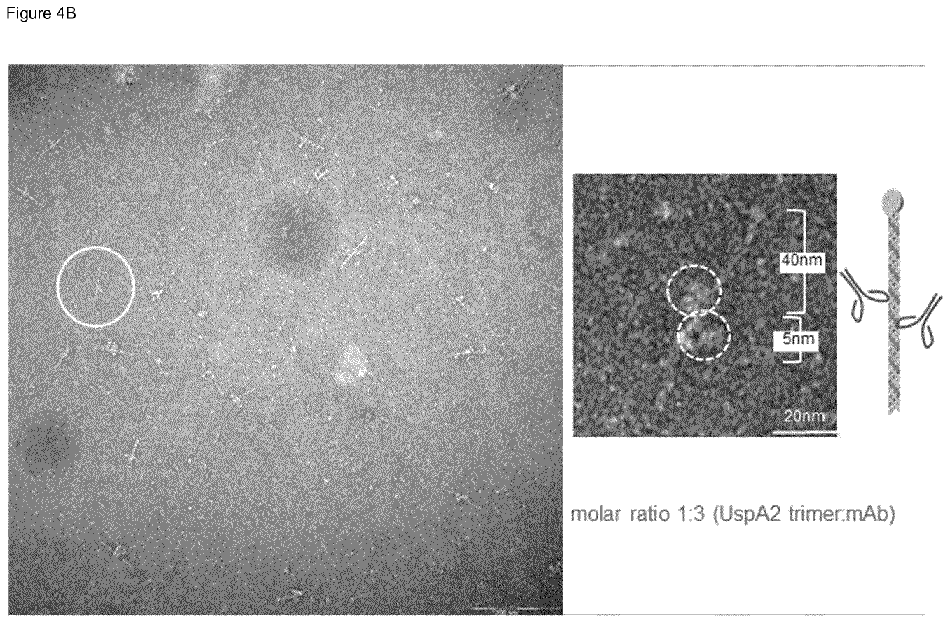

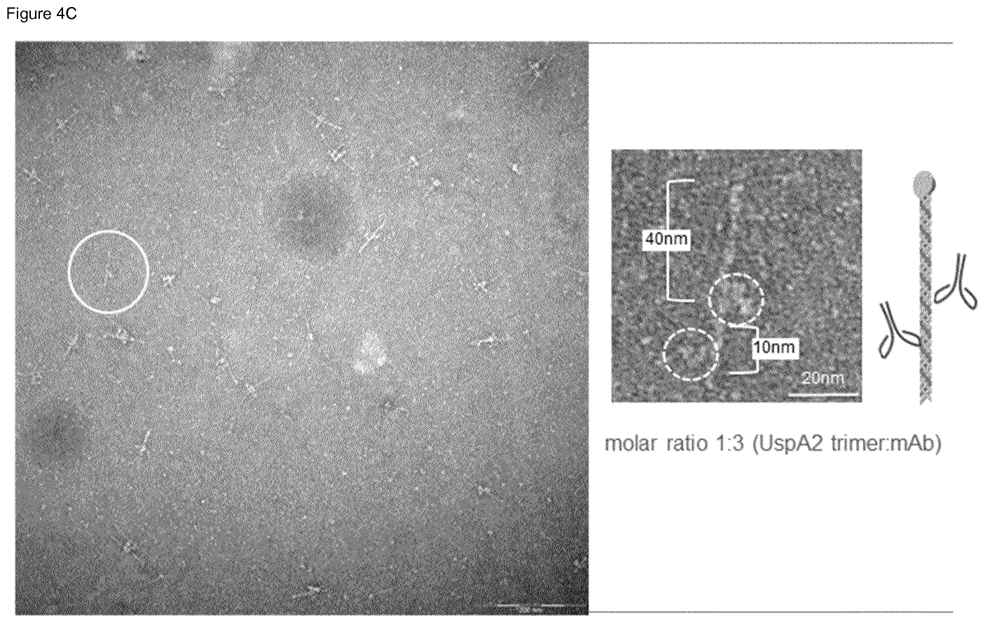

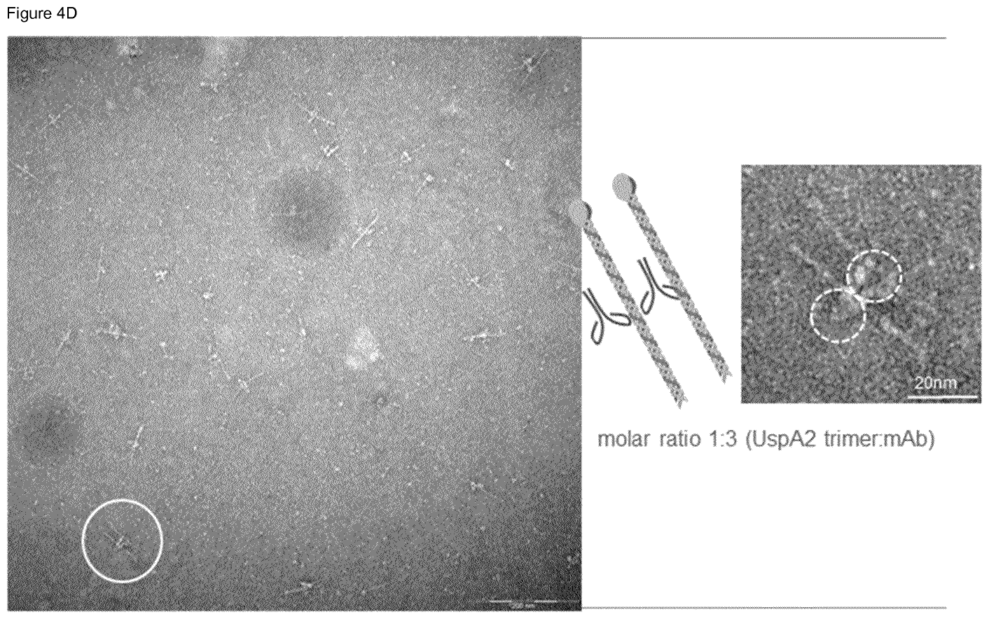

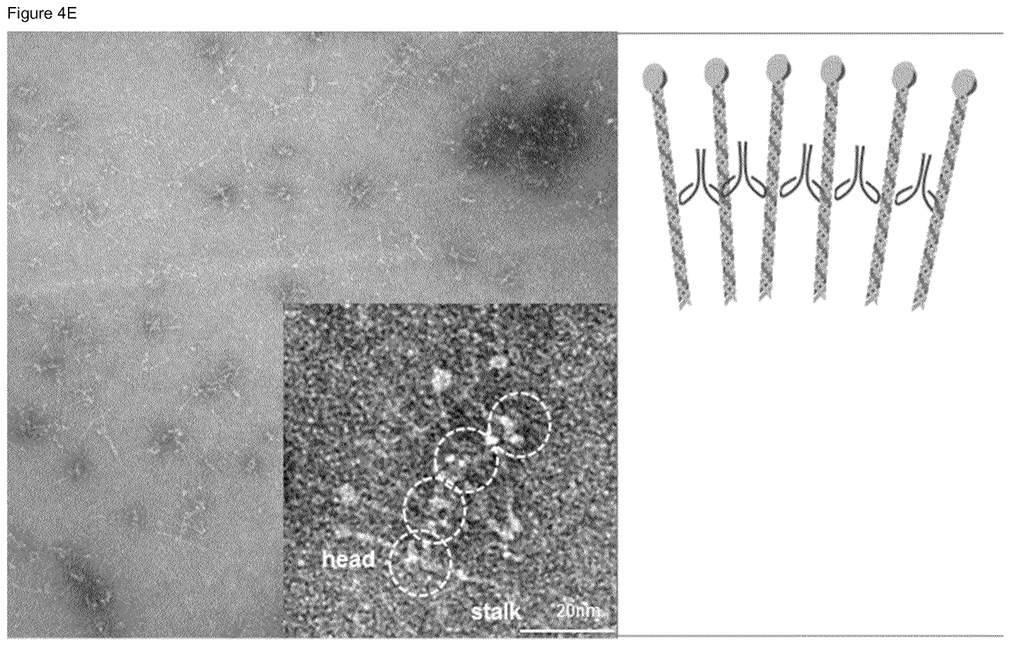

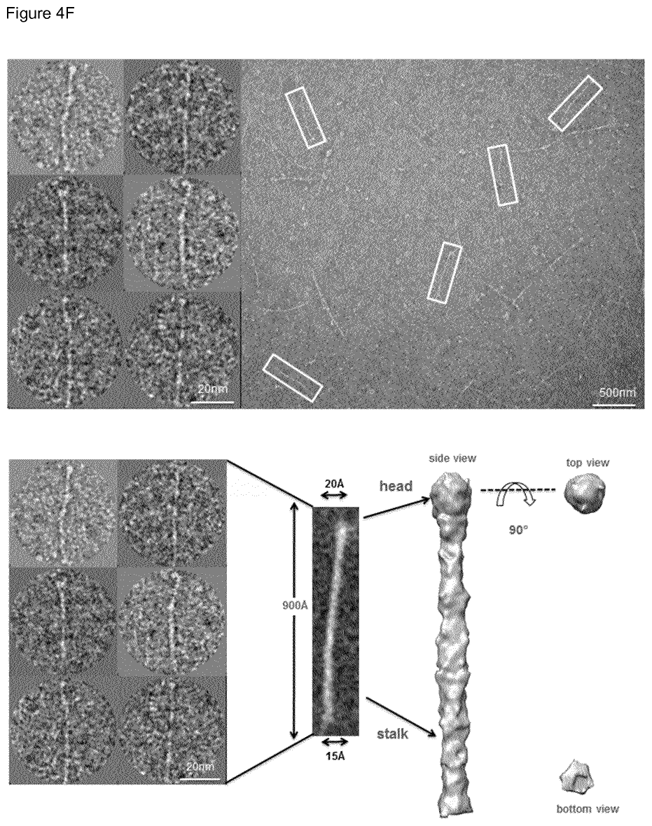

Findings demonstrated that FHUSPA2/10 binds to both the primary motif of UspA2 (determined by HDX-MS as shown in Example 3 below) as shown in FIG. 4A. At higher concentrations, the FHUSPA2/10 mAB is able to bind secondary motifs up/downstream of the primary motif (FIGS 4B+C). rograph demonstrating that at molar ratios of 1:1 (UspA2 trimer: FHUSPA2/10), FHUSPA2/10 binds to the primary motif.

Furthermore, FIG. 4D demonstrates that single FHUSPA2/10 mAbs were able to bind to the primary motifs of multiple UspA2 trimers and promote intermolecular bridging (FIG. 4D+E)

Ultrastructure of Soluble UspA2 (SEQ ID NO: 53) Molecule

The soluble UspA2 recombinant protein corresponding to the FL vaccine construct (without the leader and the anchor domains) was purified by Ni-affinity chromatography and by size-exclusion chromatography. The quality of the final UspA2 sample (SEQ ID NO: 53) was checked by SDS-PAGE and by size-exclusion analytical chromatography.

These analyses demonstrated that purified UspA2 forms homotrimers. Negative Stain electron microscopy analysis on purified UspA2 showed individual lollipop-shaped molecules with an overall length of ˜40 nm, consisting of elongated rod-like structure (˜1.5 nm thick) ending in a globular domain of ˜2 nm diameter (FIG. 4A-4C).

Structure Determination of the Straight UspA2 SEQ ID NO: 53 Molecule Alone

To gain information on the molecular architecture of the UspA2 soluble protein negative stain Transmission Electron Microscopy (TEM) images were collected and rod-like straight particles with overall length of ˜40 nm were picked out automatically and aligned by the standard procedure of Multi-Reference Alignment (MRA) and classification. The 2D class averages confirmed that UspA2 assumes a lollipop-shaped structure with dimensions of 400 Å as total length, composed by an elongated and thin stalk connected to a globular head region. Even in the presence of a small degree of heterogeneity of the particles due to their bent in the region of the elongated stalk, 2000 straight lollipop-shaped particles were selected, corrected for Contrast Transfer Function (CTF), and subjected to multiple rounds of standard MRA and classification.

We used one single particle for a preliminary C3 reconstruction. The 2D re-projections were used as initial reference to align individual UspA2 particles selected from TEM images. By knowing that the UspA2 had been purified as a homotrimer we applied a 3-fold symmetry to the first round of refinement only. After several rounds of refinement, a final 3D reconstruction was generated. The resolution was estimated as 40 Å by the FSC=0.5 criteria. Although the resolution of the map was not sufficient by itself to distinguish the atomic details, we could appreciate the overall architecture and dimensions of the UspA2 protein and were able to identify the shape of the individual components: the head and the elongated stalk. The molecule with a total length of 393 Å, includes the 370 Å long stalk, that shows a diameter of 15 Å, and is attached to the globular head of 25 Å in diameter and 20 Å in height.

### Material and Methods

Negative Staining of UspA2 (SEQ ID NO: 53) soluble protein and immuno complexes UspA2 with FHUSPA2/10.

A fraction of the purified UspA2 was diluted to 0.05 mg/ml in 20 mM Tris, 150 mM NaCl, pH 8 and loaded onto a 400-square mesh grid of carbon/formvar (Agar Scientific) glow discharged at 15 mA for 20 secs using a discharge Quorum Q150AS. The excess of the solution was blotted off using Whatman® filter Paper No. 1 (SIGMA-Aldrich) and the grid was negatively stained with 1% of Phospho Tungestinc Acid (PTA) in water for 30 seconds. Excess of stain was wicked off with Whatman® filter Paper No. 1 (SIGMA-Aldrich). The specimen was imaged using a Tecnai G2 Spirit working at 120 kV with a side mount Olympus Morada 2K×4K CCD camera and 105000× nominal magnification. The calibrated pixel size was 3.8 Å/pixel.

### Image Analysis and Structure Determination of UspA2 Soluble Protein

The micrographs collected were screened to proceed with the 3D structure generation using Imagic 5 software. The power spectra of the entire dataset were evaluated, and micrographs selected were free of drift and astigmatism. The particles in the images presented a clear preferred orientation with only lateral views, due probably to the length of the molecule that exceeds the ice thickness. Around 2000 particles were automatically selected and boxed into 300×300 pixel frames. All collected particles after rotational and translational alignment against a cylinder of the same length and thickness. Only the straight particles having identical length corresponding to the full-length homotrimer (395 Å) were selected for the next steps of Multi-Reference Alignment (MRA) and classification. A final subset of 1800 particles was created. Due to the difficult alignment of the particles that present a long, thin and flexible stalk, several cycles of rotational, translational and centering were performed. All the pre-aligned particles were than masked with a narrow rectangular mask and used in MSA to generate class averages. The best class averages were then chosen and its Euler angles set to values α=0, β=90 and γ=0 as a single orientation of a rod-like structure. This was not problematic as it will likely be able to rotate on its cylindrical axis freely, and therefore fill in all of Fourier space. A preliminary 3D reconstruction was then generated starting from the single class average with the newly set Euler angles, a point-group symmetry C3 was applied. The first map generated resembled to a star-like reconstruction due to the C3 symmetry applied and to the limited number of images present in the class average. To remove the unwanted outer parts of the density we preceded by masking each 2D section of the 3D map separately using the same mask value used previously. From the masked map an anchor set of images has then been generated by projecting the 3D density and used in the angular-reconstitution to find out the Euler angles values. Images with incorrect Euler angles (p angle too far from 90°) were removed and a new 3D reconstruction was calculated. The process was iteratively repeated adding at each cycle an increased number of good particles, until a clean and meaningful 3D map was obtained at a resolution determined to a resolution of 40 Å

### Example 3—Epitope Mapping by HDX-MS

### Materials

Deuterium oxide (99.9% D atoms), sodium deuteroxide, deuterium chloride, acetonitrile and Glu-fibrinogen peptide (GFP) were all purchased from Sigma-Aldrich and used without further purification. Poroszyme immobilised pepsin column was purchased from Thermo-Fisher.

### Methods

### Sample Preparation for HDX-MS Analyses.

1. The antibody/antigen complex was formed by adding 378 pmoles (pico moles) of UspA2 (SEQ ID NO: 53) monomer to the FHUSPA2/10 antibody using a molar ratio UspA2 monomer/mAb of 1:0.33 (or expressed as 3:1 UspA2: mAB) or 1:1 and incubated for 30 min at 25° C.

2. The labelling was initiated by adding deuterated PBS buffer (pH 7.3), reaching a deuterium excess of 87.3%, at 25° C. Over the time course of the experiment (ranging from 30 secs to 24 hours), 30 μL of the sample were removed and quenched with the same volume of an ice-cold quenching buffer (7M urea, 400 mM GuCl, 800 mM TCEP, 0.1% F.A., pH 2.4) to dissociate the antibody/antigen complex and to lower the pH to 2.4. The quenched aliquots were immediately frozen in liquid nitrogen and stored at −80° C. for less than 24 h.

A control experiment without antibody was prepared using the same conditions previously described (PBS was used instead of the antibody preparation). Labelled samples were immediately flash frozen in liquid nitrogen and stored at −80° C. for less than 24 h.

### Local HDX-MS Analyses

Labelled samples were thawed rapidly to 0° C. and injected into a Waters nanoACQUITY UPLC with HDX Technology. The injector, switching valve, columns, solvents and all associated tubings were at 0° C. to limit back-exchange. For local HDX-MS, protein samples were on-line digested for 2.5 min at 20° C. with a flow rate of 200 μL/min using a Poroszyme Immobilized Pepsin Cartridge (2.1 mm×20 mm, Thermo-Fisher) equilibrated with 100% buffer A (2% acetonitrile, 0.1% formic acid in water). The generated peptides were immediately trapped, concentrated and desalted using a VanGuard BEH Pre-column (1.7 μm, 2.1×5 mm, Waters). The 2.5 min digestion and desalting step allows deuterons located at fast exchanging sites (i.e. side chains and amino/carboxy terminus) to be replaced with hydrogens. Peptides were then separated on an ACQUITY UPLC BEH C18 reverse phase column (1.7 μm, 1.0×100 mm, Waters) with a linear gradient from 10 to 40% buffer B (2% water, 0.1% formic acid in acetonitrile) over 6.8 min at 40 μL/min.

Mass spectra acquisition: Mass spectra were acquired in resolution mode (m/z 300-2000) on a Waters SynaptG2 mass spectrometer equipped with a standard ESI source. The mass spectrometer SynaptG2 is calibrated before each analysis with a Caesium iodide solution (2 mg\mL in 50% isopropanol) infused through the reference probe of the ESI source. Mass accuracy was ensured by continuously infusing a GFP solution (600 fmol/μL in 50% acetonitrile, 0.1% formic acid) through the reference probe of the ESI source. The identity of each peptide was confirmed by MS^{E} analyses. MS^{E} was directly performed by a succession of low (6 V) and high collision (25 V) energies in the transfer region of the mass spectrometer. All fragmentations were performed using argon as collision gas. Data were processed using Protein Lynx Global Server 2.5 (Waters) and each fragmentation spectrum was manually inspected to confirm the assignment. The DynamX software (Waters) was used to select the peptides considered for the analysis and to extract the centroid mass of each of them, and for each charge state, as a function of the labelling time. Only the peptic peptides present in at least four over five repeated digestions of the unlabelled proteins were considered for the analysis.

### Synapt G2 Settings:

### Source: ES+

### Capillary: 3000 V

### Sample Cone: 25 V

### Extraction Cone: 4 V

### Source Temperature: 80° C.

### Cone gas: 20 L/h

### Results

The epitope mapping of the UspA2 (SEQ ID NO: 53) protein with the FHUSPA2/10 antibody was performed using the Waters nanoACQUITY UPLC with HDX Technology and DynamX software.

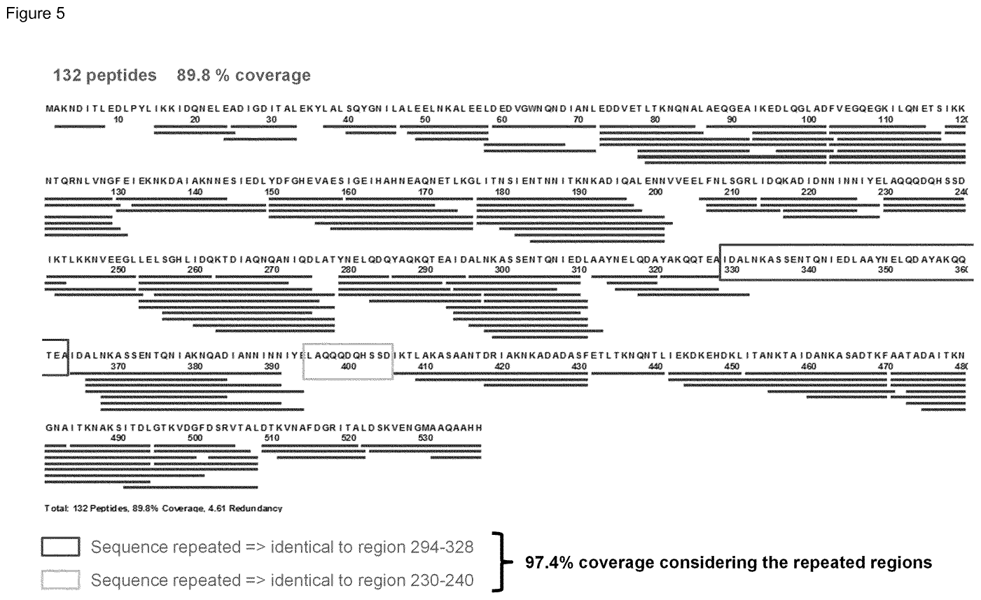

132 pepsin peptides, corresponding to 89.8% of the UspA2 sequence were considered for this analysis (FIG. 5). In the protein are present repeated regions with the same aminoacidic sequence, in particular region 294-328 is identical to region 329-363 and region 230-240 is identical to region 395-405. Considering the repeated regions, the overall sequence coverage is 97.4%.

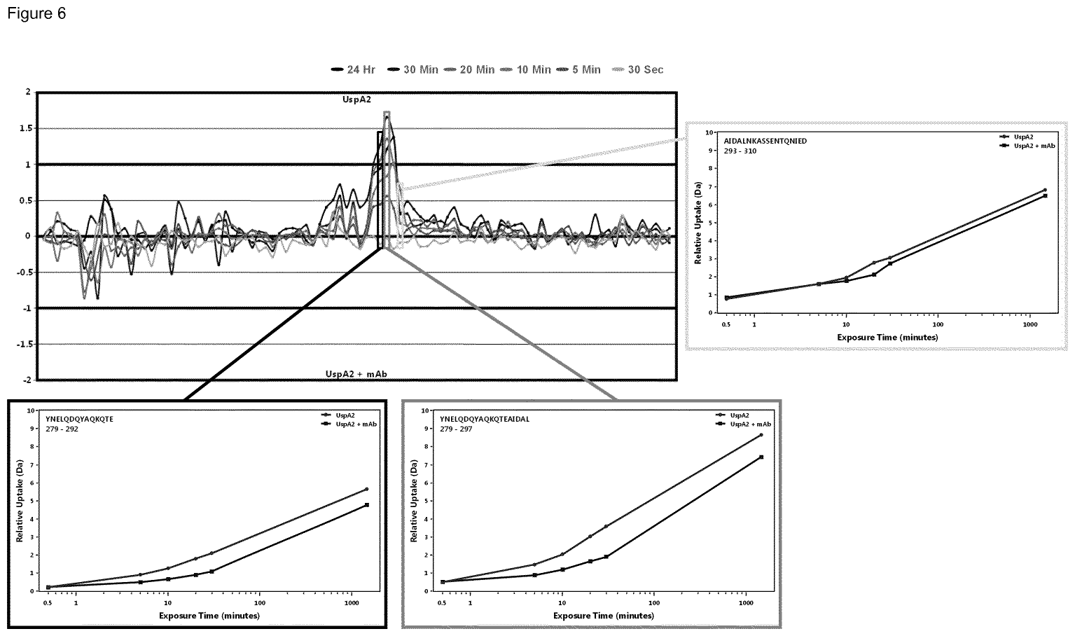

The deuterium incorporation on these 132 peptides generated from the antigen under its free or mAb-bound form with a molar ratio of 1:0.33 can be visualised in FIG. 6. The difference of deuterium incorporation was considered significant when the averaged value of deuterium incorporation is superior to 1 Da.

Peptides 279-292 and 279-297 showed a significant difference in deuterium uptake in presence of the mAb. By considering the incorporation of other peptides in the same region that does not show differences, like peptide 293-310, it is possible to reduce the epitope sequence to peptide 279-292 (YNELQDQYAQKQTE). This sequence is part of a domain repeated three times in the UspA2 (SEQ ID NO: 53) with some little amino acid differences. The differences with the other two repeated regions consist in the substitution of Q with A and the inversion of QKQ into KQQ. These residues seem to be key players of the specificity.

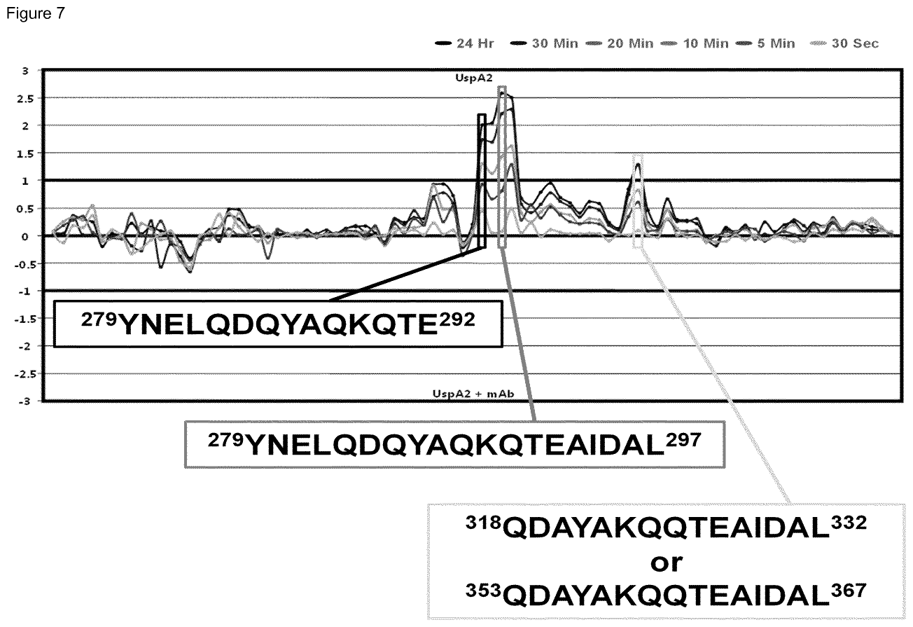

Increasing the amount of mAb compared to the protein (protein monomer/antibody molar ratio of 1:1), will result in a binding also in the repeated regions as reported in FIG. 7.

### Example 4: FHUSPA2/10 Monoclonal Antibody Cross-Bactericidal Activity

### Bactericidal Assay

Moraxella catarrhalis was cultivated overnight on Petri dish at 37° C.+5% CO₂. Bacteria were transferred in 12 ml HBSS-BSA (Hank's Buffered Salt Solution with Bovine Serum Album) 0.1% buffer in order to get an OD₆₂₀ of 0.650. Serum samples were heated for 45 min at 56° C. to inactivate the endogenous complement. Serial two-fold dilutions of sera in serum bactericidal assay (SBA) buffer (HBSS-BSA 0.1%) were added on a 96-well round bottom microtiter plate (25 μl/well). Subsequently, 50 μl of SBA buffer were added in each well. Then 25 μl of Moraxella catarrhalis strains at 4.104 CFU (colony forming unit)/ml were added to the wells containing sera and incubated for 15 min at room temperature. Finally, 25 μl of freshly thawed baby rabbit complement diluted 1/8 in HBSS-BSA 0.1% were added to reach a final volume of 125 μl. Plates were incubated for 1 h at 37° C. with orbital shaking (210 rpm). The reaction was stopped by laying the microplate on ice for at least 5 min.

After homogenization, various dilutions of the suspension (a mixture of bacteria, serum, complement and buffer, at a volume of 125 μl as discussed in the previous paragraph) were added onto chocolate agar plates and incubated for 24 hours at 37° C. with 5% CO₂ and Moraxella catarrhalis colonies were counted.

Eight wells without serum sample were used as bacterial controls to determine the number of Moraxella catarrhalis colonies per well. The mean number of CFU of the control wells was determined and used for the calculation of the killing activity for each serum sample. The bactericidal titers were expressed as the reciprocal dilution of serum inducing 50% of killing (mid-point titer).

### Results

The anti-UspA2 monoclonal antibody FHUSPA2/10 was tested in the bactericidal assay described here above against 8 different Moraxella catarrhalis strains isolated in various countries (UK, Denmark, Netherlands), that are representative of UspA2 variability.

As shown below (table 1) the anti-UspA2 monoclonal antibody FHUSPA2/10 was able to induce a cross-bactericidal killing of Moraxella catarrhalis, whatever the percentage of homology of the UspA2 expressed by the tested strain. Moreover, bactericidal activity was also shown against strains expressing the chimeric protein UspA2H. As expected, no bactericidal activity was measured against an UspA1/UspA2 double knock-out mutant.

TABLE 1
Cross-bactericidal activity of the anti-UspA2 monoclonal antibody FHUSPA2/10

| | | | | Identity %* | FHUSPA2/10 |
|---|---|---|---|---|---|
| | | | | versus the | monoclonal |
| | | | | vaccine | antibody |
| | | | | sequence | bactericidal |
| | | | UspA gene | ATCC 25238 | activity |
| M.catarrhalis strains | | Source | present | (SEQ ID NO: 1) | (mid-point titer) |
| ATCC | 25238 | Not known | UspA1/UspA2 | 45.2/100 | 1881667 |
| | 43617 | Sputum from a | UspA1 | 40.1 | <100 |
| | | Chronic | | | |
| | | Bronchitis and | | | |
| | | COPD Patient | | | |
| UK | 296 | Sputum from a | UspA1/UspA2H | 40.8/64.4 | 113906 |
| isolates | | COPD patient | | | |
| (AERIS | 415 | Sputum from a | UspA1/UspA2H | 37.1/64.6 | 256441 |
| study) | | COPD patient | | | |
| | 268 | Sputum from a | UspA1/UspA2H | 35.8/57.3 | >384000 |
| | | COPD patient | | | |
| | 240 | Sputum from a | UspA2H | 68.4 | >25600 |
| | | COPD patient | | | |
| | 193 | Sputum from a | UspA1/UspA2H | 47.1/72.5 | >640000 |
| | | COPD patient | | | |
| Dutch | F10 | Sputum from a | UspA1/UspA2 | 42.9/61.1 | 527667 |
| isolate | | patient with a | | | |
| | | lower respiratory | | | |
| | | tract infection | | | |
| Denmark | BBH18 | Sputum from a | UspA1/UspA2 | 42.9/57.3 | 385784 |
| isolate | | COPD patient | | | |
| | | with exacerbation | | | |
| | BBH18 | Mutated form of | No UspA gene | / | <100 |
| | UspA1 & | BBH18 | | | |
| | UspA2 | | | | |
| | double | | | | |
| | knock out | | | | |

*determined using the software GapL/C lustalX

### Example 5: Sequencing of the Hybridoma-Secreted Antibody FHUSPA10/10 Clone

Aim: To obtain the nucleic and amino acid sequence of hybridoma-secreted antibody of FHUSPA2-10 clone. The whole procedure aimed to sequence exclusively the variable regions of the light and heavy antibody chains (VL and VH). The sequencing strategy was designed to also obtain the sequence of a small region of the constant region (˜50-60 bp) for confirmation of the antibody class/subtype

Methods: The whole procedure can be summarized as follows:
1. Thawing and growth of hybridoma cell clone
2. RNA extraction
3. cDNA generation by retro-transcription
4. 3′ polyA tailing
5. 5′ Rapid Amplification of cDNA Ends (RACE) PCR
6. Cloning into commercial plasmid (TOPO PCR cloning)
7. Colony picking, bacterial growth and plasmid extraction
8. Sanger sequencing

Briefly, hybridoma cells were thawed and grown for 10 days;

Thawing of cells: 15 ml Falcon tubes were prepared containing 10 ml of warm DMEM medium. Cells were thawed by placing the cryotube rapidly in a 37° C. water bath. Cells were transferred into the Falcon tube and centrifuged for 10 minutes at 1000 rpm. The supernatant was carefully poured away and the cells were resuspended with 10 ml of warm D-MEM and re-centrifuged for 10 minutes at 1000 rpm. In the meanwhile, 1 ml of thawing medium was transferred into each well of the first row of a 24-well plate.

The supernatant was again poured away and the cells were resuspended with 1 ml of warm thawing media. The resuspension was transferred into the first well, mixed by gentle pipetting and then 1 ml was transferred into the near well. This 1:1 dilution was continued untill the last well. 1 ml of media was added to each well, to reach 2 ml of cell culture in each well.

The plate was placed in an incubator at 37° C. with a 5% CO2 atmosphere.

After few days, the cells were transferred from the well where they are not fully convergent into a T25 flask for adherent cells adding fresh thawing media to 10 ml total volume.

### Cell Subculturing Protocol

Cells were recovered by shaking gently the T25 flask and pouring the resuspension into a 50 ml Falcon tube. The tube was then centrifuged for 10 minutes at 1000 rpm, and the pellet resuspend in warm D-MEM. This process was repeated however the second resuspension step was conducted in a 50 ml of warm growing medium. The cells were then transferred into a T75 flask.

RNA was then extracted (4 samples of cells, each containing 7×10⁶ cells) using the Qiagen RNeasy Mini kit (according to manufacturers instructions) followed by cDNA generation by retro transcription of 4.5 μg RNA. Retro transcription was performed using SuperScript IV first-strand synthesis system (Invitrogen) and a set of oligos specific for either the light chain or heavy chain amplification:

HEAVY CHAIN oligos (for all isotypes):
RT-mHingeG1_rev
5′-gcaaggcttacaaccacaatc-3′
RT-mHingeG2a_rev
5′-gaggacagggcttgattgtgg-3′
RT-mHingeG2b_rev
5′-gaggacaggggttgattgttg-3′
RT-mHingeG2c_rev
5′-ggacaggggttctgtgttatgg-3′
RT-mHingeG3_rev
5′-ctgggcttgggtattctagg-3′
LIGHT CHAIN oligos (for both kappa and lambda
classes):
RT_mKappaCL_rev
5′-ctcattcctgttgaagctcttg-3′
RT_mLambda1/4-CL_rev
5′-gcacgggacaaactcttctc-3′
RT_mLambda2/3-CL_rev
5′-ctgcaggagacagactcttctc-3′

3′ polyA tailing was performed using between 680 and 200 ng of cDNA and Terminal Deoxynucleotidyl Transferase (ThermoScientific) and dATP (Invitrogen). This generated (after column purification) 400-800 ng of polyA cDNA, following which 5′ rapid amplification of cDNA ends (RACE) PCR was performed using either Q5 Hot Start polymerase (NEB) or Platinum SuperFi polymerase (Invitrogen), and a set of oligos specific for either the light chain or heavy chain amplification:

HEAVY CHAIN oligos (for all isotypes):
TAR2_RACE-5E_mG1_rev
5′-GTCTCGTGGGCTCGGAGATGTGTATAAGAGACAGgttagtttgggcag
cagatc-3′
TAR2_RACE-5E_mG2a.2c_rev
5′-GTCTCGTGGGCTCGGAGATGTGTATAAGAGACAGgaggagccagttgt
ayctc-3′
TAR2_RACE-5E_mG2b_rev
5′-GTCTCGTGGGCTCGGAGATGTGTATAAGAGACAGgaaccagttgtatc
tccacac-3′
TAR2_RACE_5E_G3_rev
5′-GTCTCGTGGGCTCGGAGATGTGTATAAGAGACAGgatccagatgtgtc
actgc-3′
LIGHT CHAIN oligos (for both kappa and lambda
classes):
TAR2_RACE-5E_mKappaCL_rev
5′-GTCTCGTGGGCTCGGAGATGTGTATAAGAGACAGgtgggaagatggat
acagttg-3′
TAR2_RACE-5E_mLambda1/4_rev, Tm 65° C.
5′-GTCTCGTGGGCTCGGAGATGTGTATAAGAGACAGgagctcttcagagg
aaggtg-3′
TAR2_RACE-5E_mLambda2/3_rev
5′-GTCTCGTGGGCTCGGAGATGTGTATAAGAGACAGctcagrggaaggtg
gaaac-3′

A common forward oligo was used:

TAR1_XSCTnTag_V3.17
5′-Tcgtcggcagcgtcagatgtgtataagagacagttttttttttttttt
tt-3′

Cloning into commercial plasmid (and transofmration) was performed using ZeroBlunt TOPO PCR Kits according to manufacturers instructions (Invitrogen). Colonies were then picked and plasmids extracted using the Qiaprep Miniprep Kit (Qiagen) and Sanger Sequences was performed.

100 ng of plasmid was used and the following oligos:

M13 Forward
5′-GTAAAACGACGGCCAG-3′
M13 Reverse
5′-CAGGAAACAGCTATGAC-3′

QiAquick Gel Extraction Kit and MinElute PCR Purification Kit (Qiagen) were used for the DNA purification steps.

### Analysis of Heavy Chain Sequencing

Upon analysis, the sequences obtained for every tested clone share the following regions organization;
polyT sequence
5′ UTR (UnTranslated Region)+leader
Full variable heavy (VH) domain
Short region of CH1 domain
NGS adapters (5/3′) [needed only if DNA submitted to NGS sequencing]

Using a sequence analysis software (DNASTAR LaserGene 12), everything was discarded but the antibody genes (VH and CH1). Upon alignment, all sequences show complete homology. Table 2 lists the features of the sequence obtained.

TABLE 2
VH Sequence

| | | |
|---|---|---|
| VH Sequence | VH Nucleic | GAGGTTCAGCTCCAGCAGTCTGGG |
| Analysis | Acid (SEQ ID | ACTGTGCTGGCAAGGCCTGGGGCT |
| | NO: 81) | TCCGTGAAGATGTCCTGCAAGGCT |
| | | TCTGGCTACAGCTTTACCAGCTAC |
| | | TGGATGCACTGGGTAAAACAGAGG |
| | | CCTGGACAGGGTCTAGAATGGATT |
| | | GGTGCTATTTATCCTGGAAATAGT |
| | | GATACTAGCTACAACCAGAAGTTC |
| | | AAGGGCAAGGCCAAACTGACTGCA |
| | | GTCACATCCGCCAGCACTGCCTAC |
| | | ATGGAGCTCAGCAACCTGACGAAT |
| | | GAGGACTCTGCGGTCTATTCCTGT |
| | | ACATTACTACGTTTCCTCGATGCT |
| | | TACTGGGGCCAAGGGACTCTGGTC |
| | | ACTGTCTCTGCAG |
| | VH Amino | EVQLQQSGTVLARPGASVKMSCKA |
| | Acid (SEQ ID | SGYSFTSYWMHWVKQRPGQGLEWI |
| | NO: 82) | GAIYPGNSDTSYNQKFKGKAKLTA |
| | | VTSASTAYMELSNLTNEDSAVYSC |
| | | TLLRFLDAYWGQGTLVTVSA |

### Analysis of Light Chain Sequencing

TABLE 3 summarizes the data for th
eproductive light chain.

| | | |
|---|---|---|
| VL Sequence | VL Nucleic | CAAATTGTTCTCTCCCAGTCTCCA |
| Analysis | Acid (SEQ ID | GCAATCCTGTCTGCATCTCCAGGG |
| | NO: 83) | GAGAAGGTCACAATGACTTGCAGG |
| | | GCCAGCTCAAGTGTAAGTTACATG |
| | | CACTGGTACCAGCAGAAGCCAGGA |
| | | TCCTCCCCCAAACCCTGGATTTAT |
| | | GCCACATCCAACCTGGCTTCTGGA |
| | | GTCCCTGCTCGCTTCAGTGGCAGT |
| | | GGGTCTGGGACCTCTTACTCTCTC |
| | | ACAATCAGCAGAGTGGAGGCTGAA |
| | | GATGCTGCCACTTATTACTGCCAG |
| | | CAGTGGAGTAGTAACCCACCCACG |
| | | TTCGGAGGGGGGTCCAAGCTGGAA |
| | | ATAAAA |
| | VL Amino | QIVLSQSPAILSASPGEKVTMTCR |
| | Acid (SEQ ID | ASSSVSYMHWYQQKPGSSPKPWIY |
| | NO: 84) | ATSNLASGVPARFSGSGSGTSYSL |
| | | TISRVEAEDAATYYCQQWSSNPPT |
| | | FGGGSKLEIK |
| | CL | Kappa class |

Two aberrant transcripts were also identified (aberrant transripts k138 and k142) as described in Cabilly and Riggs, Gene. 1985; 40(1):157-61. The aberrant chains are likely not contributing to any binding activity.

### CONCLUSIONS

The VH and VL antibody genes of hybridoma clone FHUSPA2/10 were successfully amplified and sequenced.
Isotype of FHUSPA2/10 antibody was confirmed as IgG2a.

### REFERENCES

1. Hoiczyk E, Roggenkamp A, Reichenbecher M, Lupas A, Heesemann J. Structure and sequence analysis of Yersinia YadA and Moraxella UspAs reveal a novel class of adhesins. EMBO J. 2000; 19(22):5989-99.
2. Aebi C, Maciver I, Latimer J L, Cope L D, Stevens M K, Thomas S E, et al. A protective epitope of Moraxella catarrhalis is encoded by two different genes. Infect Immun. 1997; 65(11):4367-77.
3. Helminen M E, Maciver I, Latimer J L, Klesney-Tait J, Cope L D, Paris M, et al. A large, antigenically conserved protein on the surface of Moraxella catarrhalis is a target for protective antibodies. J Infect Dis. 1994; 170(4):867-72.
4. Cope L D, Lafontaine E R, Slaughter C A, Hasemann C A, Jr., Aebi C, Henderson F W, et al. Characterization of the Moraxella catarrhalis uspA1 and uspA2 genes and their encoded products. J Bacteriol. 1999; 181(13):4026-34.
5. Tan T T, Nordstrom T, Forsgren A, Riesbeck K. The respiratory pathogen Moraxella catarrhalis adheres to epithelial cells by interacting with fibronectin through ubiquitous surface proteins A1 and A2. J Infect Dis. 2005; 192(6):1029-38.
6. Tan T T, Forsgren A, Riesbeck K. The respiratory pathogen Moraxella catarrhalis binds to laminin via ubiquitous surface proteins A1 and A2. J Infect Dis. 2006; 194(4):493-7.
7. Attia A S, Lafontaine E R, Latimer J L, Aebi C, Syrogiannopoulos G A, Hansen E J. The UspA2 protein of Moraxella catarrhalis is directly involved in the expression of serum resistance. Infect Immun. 2005; 73(4):2400-10.
8. de Vries S P, Bootsma H J, Hays J P, Hermans P W. Molecular aspects of Moraxella catarrhalis pathogenesis. Microbiol Mol Biol Rev. 2009; 73(3):389-406, Table of Contents.
9. Perez A C, Murphy T F. Potential impact of a Moraxella catarrhalis vaccine in COPD. Vaccine. 2017.
10. Sapey E, Stockley R A. COPD exacerbations. 2: aetiology. Thorax. 2006; 61(3):250-8.
11. Erb-Downward J R, Thompson D L, Han M K, Freeman C M, McCloskey L, Schmidt L A, et al. Analysis of the lung microbiome in the “healthy” smoker and in COPD. PLoS One. 2011; 6(2):e16384.
12. Wilkinson T M A, Aris E, Bourne S, Clarke S C, Peeters M, Pascal T G, et al. A prospective, observational cohort study of the seasonal dynamics of airway pathogens in the aetiology of exacerbations in COPD. Thorax. 2017; 72(10):919-27.
13. Sethi S, Evans N, Grant B J, Murphy T F. New strains of bacteria and exacerbations of chronic obstructive pulmonary disease. N Engl J Med. 2002; 347(7):465-71.
14. Alamoudi O S. Bacterial infection and risk factors in outpatients with acute exacerbation of chronic obstructive pulmonary disease: a 2-year prospective study. Respirology. 2007; 12(2):283-7.
15. Bandi V, Jakubowycz M, Kinyon C, Mason E O, Atmar R L, Greenberg S B, et al. Infectious exacerbations of chronic obstructive pulmonary disease associated with respiratory viruses and non-typeable Haemophilus influenzae. FEMS Immunol Med Microbiol. 2003; 37(1):69-75.
16. Beasley V, Joshi P V, Singanayagam A, Molyneaux P L, Johnston S L, Mallia P. Lung microbiology and exacerbations in COPD. Int J Chron Obstruct Pulmon Dis. 2012; 7:555-69.
17. Ren D, Pichichero M E. Vaccine targets against Moraxella catarrhalis. Expert Opin Ther Targets. 2016; 20(1):19-33.
18. Holliger P, Hudson P J. Engineered antibody fragments and the rise of single domains. Nat Biotechnol. 2005; 23(9):1126-36.
19. WO2007/018463.
20. Hallstrom T, Nordstrom T, Tan T T, Manolov T, Lambris J D, Isenman D E, et al. Immune evasion of Moraxella catarrhalis involves ubiquitous surface protein A-dependent C3d binding. J Immunol. 2011; 186(5):3120-9.
21. Brooks M J, Sedillo J L, Wagner N, Laurence C A, Wang W, Attia A S, et al. Modular arrangement of allelic variants explains the divergence in Moraxella catarrhalis UspA protein function. Infect Immun. 2008; 76(11):5330-40.
22. Hill D J, Whittles C, Virji M. A novel group of Moraxella catarrhalis UspA proteins mediates cellular adhesion via CEACAMs and vitronectin. PLoS One. 2012; 7(9):e45452.
23. WO2015/125118A1.
24. Rice P, Longden I, Bleasby A. EMBOSS: the European Molecular Biology Open Software Suite. Trends Genet. 2000; 16(6):276-7.
25. Needleman S B, Wunsch C D. A general method applicable to the search for similarities in the amino acid sequence of two proteins. J Mol Biol. 1970; 48(3):443-53.
26. Henikoff S, Henikoff J G. Amino acid substitution matrices from protein blocks. Proc Natl Acad Sci USA. 1992; 89(22):10915-9.
27. Eric P. Dixon SSaKEK. Cell Biology (Third Edition) A Laboratory Handbook: Chapter 58—Rapid Development of Monoclonal Antibodies Using Repetitive Immunizations, Multiple Sites.

Academic Press. 2006; 1:483-90.
28. Su Y C, Hallstrom B M, Bernhard S, Singh B, Riesbeck K. Impact of sequence diversity in the Moraxella catarrhalis UspA2/UspA2H head domain on vitronectin binding and antigenic variation. Microbes Infect. 2013; 15(5):375-87.
29. Lafontaine E R, Cope L D, Aebi C, Latimer J L, McCracken G H, Jr., Hansen E J. The UspA1 protein and a second type of UspA2 protein mediate adherence of Moraxella catarrhalis to human epithelial cells in vitro. J Bacteriol. 2000; 182(5):1364-73.
30. Hill D J, Edwards A M, Rowe H A, Virji M. Carcinoembryonic antigen-related cell adhesion molecule (CEACAM)-binding recombinant polypeptide confers protection against infection by respiratory and urogenital pathogens. Mol Microbiol. 2005; 55(5):1515-27.
31. Hill D J, Virji M. A novel cell-binding mechanism of Moraxella catarrhalis ubiquitous surface protein UspA: specific targeting of the N-domain of carcinoembryonic antigen-related cell adhesion molecules by UspA1. Mol Microbiol. 2003; 48(1):117-29.
32. Nordstrom T, Blom A M, Tan T T, Forsgren A, Riesbeck K. Ionic binding of C3 to the human pathogen Moraxella catarrhalis is a unique mechanism for combating innate immunity. J Immunol. 2005; 175(6):3628-36.
33. Nordstrom T, Blom A M, Forsgren A, Riesbeck K. The emerging pathogen Moraxella catarrhalis interacts with complement inhibitor C4b binding protein through ubiquitous surface proteins A1 and A2. J Immunol. 2004; 173(7):4598-606.
34. Meier P S, Troller R, Grivea I N, Syrogiannopoulos G A, Aebi C. The outer membrane proteins UspA1 and UspA2 of Moraxella catarrhalis are highly conserved in nasopharyngeal isolates from young children. Vaccine. 2002; 20(13-14):1754-60.
35. EP 0594610
36. WO91/18926
37. WO00/56360
38. Hallstrom T, Blom A M, Zipfel P F, Riesbeck K. Nontypeable Haemophilus influenzae protein E binds vitronectin and is important for serum resistance. J Immunol. 2009; 183(4):2593-601.
39. Ronander E, Brant M, Eriksson E, Morgelin M, Hallgren O, Westergren-Thorsson G, et al. Nontypeable Haemophilus influenzae adhesin protein E: characterization and biological activity. J Infect Dis. 2009; 199(4):522-31.
40. Ronander E, Brant M, Janson H, Sheldon J, Forsgren A, Riesbeck K. Identification of a novel Haemophilus influenzae protein important for adhesion to epithelial cells. Microbes Infect. 2008; 10(1):87-96.
41. Singh B, Brant M, Kilian M, Hallstrom B, Riesbeck K. Protein E of Haemophilus influenzae is a ubiquitous highly conserved adhesin. J Infect Dis. 2010; 201(3):414-9.
42. WO2007/084053.
43. WO2012/139225A1.
44. Bakaletz L O, Baker B D, Jurcisek J A, Harrison A, Novotny L A, Bookwalter J E, et al. Demonstration of Type IV pilus expression and a twitching phenotype by Haemophilus influenzae. Infect Immun. 2005; 73(3):1635-43.
45. GB2220211A.
46. Thoelen S, Van Damme P, Mathei C, Leroux-Roels G, Desombere I, Safary A, et al. Safety and immunogenicity of a hepatitis B vaccine formulated with a novel adjuvant system. Vaccine. 1998; 16(7):708-14.
47. EP689454B1.
48. WO 94/00153.
49. WO 96/33739.
50. WO 95/17210.
51. WO 96/02555.
52. WO 0226757
53. WO 03507822.
54. Cripps A W, Otczyk D C. Prospects for a vaccine against otitis media. Expert Rev Vaccines. 2006; 5(4):517-34.
55. American Academy of Pediatrics Subcommittee on Management of Acute Otitis M. Diagnosis and management of acute otitis media. Pediatrics. 2004; 113(5):1451-65.
56. Prymula R, Schuerman L. 10-valent pneumococcal nontypeable Haemophilus influenzae PD conjugate vaccine: Synflorix. Expert Rev Vaccines. 2009; 8(11):1479-500.
57. Murphy T F. Current and Future Prospects for a Vaccine for Nontypeable Haemophilus influenzae. Curr Infect Dis Rep. 2009; 11(3):177-82.
58. Pichichero M E, Casey J R. Otitis media. Expert Opin Pharmacother. 2002; 3(8):1073-90.
59. Sabirov A, Metzger D W. Mouse models for the study of mucosal vaccination against otitis media. Vaccine. 2008; 26(12):1501-24.
60. Murphy T F. Respiratory infections caused by non-typeable Haemophilus influenzae. Curr Opin Infect Dis. 2003; 16(2):129-34.
61. Buist A S, McBurnie M A, Vollmer W M, Gillespie S, Burney P, Mannino D M, et al. International variation in the prevalence of COPD (the BOLD Study): a population-based prevalence study. Lancet. 2007; 370(9589):741-50.
62. Mannino D M, Homa D M, Akinbami L J, Ford E S, Redd S C. Chronic obstructive pulmonary disease surveillance—United States, 1971-2000. Respir Care. 2002; 47(10):1184-99.
63. Mortality G B D, Causes of Death C. Global, regional, and national life expectancy, all-cause mortality, and cause-specific mortality for 249 causes of death, 1980-2015: a systematic analysis for the Global Burden of Disease Study 2015. Lancet. 2016; 388(10053):1459-544.
64. Wilkinson T M, Hurst J R, Perera W R, Wilks M, Donaldson G C, Wedzicha J A. Effect of interactions between lower airway bacterial and rhinoviral infection in exacerbations of COPD. Chest. 2006; 129(2):317-24.
65. Alcon A, Fabregas N, Torres A. Pathophysiology of pneumonia. Clin Chest Med. 2005; 26(1):39-46.
66. Takaki M, Nakama T, Ishida M, Morimoto H, Nagasaki Y, Shiramizu R, et al. High incidence of community-acquired pneumonia among rapidly aging population in Japan: a prospective hospital-based surveillance. Jpn J Infect Dis. 2014; 67(4):269-75.
67. Hartmann M D, Ridderbusch O, Zeth K, Albrecht R, Testa O, Woolfson D N, et al. A coiled-coil motif that sequesters ions to the hydrophobic core. Proc Natl Acad Sci USA. 2009; 106(40):16950-5.
68. Bourne S, Cohet C, Kim V, Barton A, Tuck A, Aris E, Media-Vela S, Devaster J M, Ballou W R, Clarke S and Wilkinson T. Acute exacerbation and respiratory infectionS in COPD (AERIS): protocol for a prospective, obervational cohort study. BMJ open. 2014; 4(3):e004546.

Sequence Listing

| Strain | UspA2 sequences |
|---|---|
| ATCC | MKTMKLLPLKIAVTSAMIIGLGAASTANAQAKNDITLEDLPYLIKKIDQNELEADIGDITALEK |
| 25238 | YLALSQYGNILALEELNKALEELDEDVGWNQNDIANLEDDVETLTKNQNALAEQGEAIKE |
| (SEQ ID | DLQGLADFVEGQEGKILQNETSIKKNTQRNLVNGFEIEKNKDAIAKNNESIEDLYDFGHEV |
| NO: 1) | AESIGEIHAHNEAQNETLKGLITNSIENTNNITKNKADIQALENNVVEELFNLSGRLIDQKAD |
| | IDNNINNIYELAQQQDQHSSDIKTLKKNVEEGLLELSGHLIDQKTDIAQNQANIQDLATYNE |
| | LQDQYAQKQTEAIDALNKASSENTQNIEDLAAYNELQDAYAKQQTEAIDALNKASSENTQ |
| | NIEDLAAYNELQDAYAKQQTEAIDALNKASSENTQNIAKNQADIANNINNIYELAQQQDQH |
| | SSDIKTLAKASAANTDRIAKNKADADASFETLTKNQNTLIEKDKEHDKLITANKTAIDANKA |
| | SADTKFAATADAITKNGNAITKNAKSITDLGTKVDGFDSRVTALDTKVNAFDGRITALDSKV |
| | ENGMAAQAALSGLFQPYSVGKFNATAALGGYGSKSAVAIGAGYRVNPNLAFKAGAAINT |
| | SGNKKGSYNIGVNYEF (630 aa) |
| American | MKTMKLLPLKIAVTSAMIIGLGAASTANAQSRDRSLEDIQDSISKLVQDDINTLKQDQQKM |
| 2933 | NKYLLLNQLANTLITDELNNNVIKNTNSIEALGDEIGWLENDIADLEEGVEELTKNQNTLIEK |
| (SEQ ID | DEEHDRLIAQNQADIQTLENNVVEELFNLSGRLIDQEADIAKNNASIEELYDFDNEVAERIG |
| NO: 2) | EIHAYTEEVNKTLENLITNSVKNTDNIDKNKADIDNNINHIYELAQQQDQHSSDIKTLKNNV |
| | EEGLLELSGHLIDQKADLTKDIKALESNVEEGLLDLSGRLLDQKADLTKDIKALESNVEEGL |
| | LDLSGRLLDQKADIAQNQTDIQDLAAYNELQDQYAQKQTEAIDALNKASSENTQNIEDLAA |
| | YNELQDAYAKQQTEAIDALNKASSENTQNIAKNQADIANNINNIYELAQQQDQHSSDIKTL |
| | AKASAANTNRIATAELGIAENKKDAQIAKAQANANKTAIDENKASADTKFAATADAITKNG |
| | NAITKNAKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNIGVNYEF |
| | (613 aa) |
| American | MKTMKLLPLKIAVTSALIIGLGAASTANAQQQLQTETFLPNFLSNDNYDLTDPFYHNMILGD |
| 2912 | TALLDKQDGSQPQLKFYSNDKDSVPDSLLFSKLLHEQQLNGFKKGDTIIPLDKDGKPVYQ |
| (SEQ ID | VDYKLDGKGKKQKRRQVYSVTTKTATDDDVNSAYSRGILGKVDDLDDEMNFLNHDITSL |
| NO: 3) | YDVTANQQDAIKDLKKGVKGLNKELKELDKEVGVLSRDIGSLNDDVAQNNESIEDLYDFS |
| | QEVADSIGEIHAHNKAQNETLQDLITNSVENTNNITKNKADIQALENNVVEELFNLSGRLID |
| | QKADLTKDIKTLESNVEEGLLELSGHLIDQKADIAKNQADIAQNQANIQDLAAYNELQDAY |
| | AKQQTEAIDALNKASSENTQNIEDLAAYNELQDAYAKQQTEAIDALNKASSENTQNIAKNQ |
| | ADIANNINNIYELAQQQDQHSSDIKTLAKASAANTDRIAKNKADADASFETLTKNQNTLIEK |
| | DKEHDKLITANKTAIDENKASADTKFAATADAITKNGNAITKNAKSITDLGTKVDGFDSRVT |
| | ALDTKVNAFDGRITALDSKVENGMAAQAALSGLFQPYSVGKFNATAALGGYGSKSAVAIG |
| | AGYRVNPNLAFKAGAAINTSGNKKGSYNIGVNYEF (644 aa) |
| American | MKTMKLLPLKIAVTSALIVGLGAASTANAQLVERFFPNIFLDKPLAKQHYHNVVVGDTSIVS |
| 2908 | DLQSNSDQLKFYSDDEGLVPDSLLFNKMLHEQLLNGFKEGDTIIPLDENGKPVYKVDYKL |
| (SEQ ID | DGKEPRKVYSVTTKIATAEDVATSSYANGIQKDIDDLYDFDHQVTERLTQHGKTIYRNGE |
| NO: 4) | RILANEESVQYLNKEVQNNIEHIYELAQQQDQHSSDIKTLESNVEKGLLELSGHLIDQKAD |
| | LTKDIKTLESNVEEGLLDLSGRLIDQKADLTKDIKTLESNVEEGLLDLSGRLIDQKADIAQN |
| | QANIQDLAAYNELQDQYAQKQTEAIDALNKASSENTQNIEDLAAYNELQDAYAKQQTEAI |
| | DALNKASSENTQNIAKNQADIANNINNIYELAQQQDQHSSDIKTLAKASAANTNRIATAELG |
| | IAENKKDAQIAKAQANANKTAIDENKASADTKFAATADAITKNGNAITKNAKSITDLGTKVD |
| | GFDSRVTALDTKVNAFDGRITALDSKVENGMAAQAALSGLFQPYSVGKFNATAALGGYG |
| | SKSAVAIGAGYRVNPNLAFKAGAAINTSGNKKGSYNIGVNYEF (591 aa) |
| Finnish 307 | MKTMKLLPLKIAVTSAMIIGLGAASTANAQQQQQQQQQQQSRTEIFFPNIFFNENHDELD |
| (SEQ ID | DAYHNIILGDTALLDKQDGSQPQLKFYSNDKDSVPDSLLFSKLLHEQQLNGFKKGDTIIPL |
| NO: 5) | DKDGKPVYQVDYKLDGKGKKQKRRQVYSVTTKTATDDDVNSAYSRGILGKVDDLDDEM |
| | NFLNHDITSLYDVTANQQDAIKGLKKGVKGLNKELKELDKEVGVLSRDIGSLNDDVAQNN |
| | ESIEDLYDFSQEVADSIGEIHAHNKAQNETLQDLITNSVENTNNITKNKADIQALENNVVEE |
| | LFNLSGRLIDQKADLTKDIKTLESNVEEGLLELSGHLIDQKADIAKNQADIAQNQANIQDLA |
| | AYNELQDAYAKQQTEAIDALNKASSENTQNIEDLAAYNELQDAYAKQQTEAIDALNKASS |
| | ENTQNIEDLAAYNELQDAYAKQQTEAIDALNKASSENTQNIAKNQADIANNINNIYELAQQ |
| | QDQHSSDIKTLAKASAANTDRIAKNKADADASFETLTKNQNTLIEKDKEHDKLITANKTAID |
| | ENKASADTKFAATADAITKNGNAITKNAKSITDLGTKVDAFDGRVTALDTKVNAFDGRITAL |
| | DSKVENGMAAQAALSGLFQPYSVGKFNATAALGGYGSKSAVAIGAGYRVNPNLAFKAGA |
| | AINTSGNKKGSYNIGVNYEF (687 aa) |
| Finnish 353 | MKTMKLLPLKIAVTSAMIVGLGMASTANAQQQKSPKTETFLPNIFFNEYADDLDTLYHNMI |
| (SEQ ID | LGDTAITHDDQYKFYADDATEVPDSLFFNKILHDQLLYGFKEGDKIIPLDENGKPVYKLDK |
| NO: 6) | RLENGVQKTVYSVTTKTATADDVNSAYSRGIQGDIDDLYEANKENVNRLIEHGDKIFANEE |
| | SVQYLNREVQNNIENIHELAQQQDQHSSDIKTLKKNVEKDLLDLSGRLIAQKEDIAQNQTD |
| | IQDLATYNELQDQYAQKQTEAIDALNKASSENTQNIAKNSNHIKTLENNIEEGLLELSGHLI |
| | DQKADLTKDIKALESNVEEGLLDLSGRLIDQKADIAQNQANIQDLAAYNELQDAYAKQQTE |
| | AIDALNKASSENTQNIEDLAAYNELQDAYAKQQTEAIDALNKASSENTQNIEDLAAYNELQ |
| | DAYAKQQTEAIDALNKASSENTQNIAKNQADIANNINNIYELAQQQDQHSSDIKTLAKASA |
| | ANTDRIAKNKADADASFETLTKNQNTLIEKDKEHDKLITANKTAIDANKASADTKFAATADA |
| | ITKNGNAITKNAKSITDLGTKVDGFDGRVTALDTKVNALDTKVNAFDGRITALDSKVENGM |
| | AAQAALSGLFQPYSVGKFNATAALGGYGSKSAVAIGAGYRVNPNLAFKAGAAINTSGNK |
| | KGSYNIGVNYEF (683 amino acids) |
| Finnish 358 | MKTMKLLPLKIAVTSAMMVGLGMASTANAQQQKSPKTEIFLPNLFDNDNTELTDPLYHNM |
| (SEQ ID | ILGNTALLTQENQYKFYADDGNGVPDSLLFNKILHDQLLHGFKEGGTIIPLDENGKPVYKL |
| NO: 7) | DSIVEQGKTKTVYSVTTKTATADDVNSAYSRGIQGDIDDLYEANKENVNRLIEHGDKIFAN |
| | EESVQYLNREVQNNIENIHELAQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGAAINTSG |
| | NKKGSYNIGVNYEF (684 amino acids) |
| Finnish 216 | MKTMKLLPLKIAVTSAMIIGLGAASTANAQQQQKTKTEVFLPNLFDNDYYDLTDPLYHSMI |
| (SEQ ID | LGDTATLFDQQDNSKSQLKFYSNDKDSVPDSLLFSKLLHEQQLNGFKAGDTIIPLDKDGK |
| NO: 8) | PVYTQDTRTKDGKVETVYSVTTKIATQDDVEQSAYSRGIQGDIDDLYDINREVNEYLKATH |
| | DYNERQTEAIDALNKASSANTDRIDTAEERIDKNEYDIKALESNVGKDLLDLSGRLIAQKED |
| | IDNNINHIYELAQQQDQHSSDIKTLKNNVEEGLLELSGHLIDQKADLTKDIKTLENNIEEGLL |
| | ELSGHLIDQKADLTKDIKTLENNIEEGLLELSGHLIDQKADIAQNQANIQDLAAYNELQDQY |
| | AQKQTEAIDALNKASSENTQNIEDLAAYNELQDAYAKQQTEAIDALNKASSENTQNIEDLA |
| | AYNELQDAYAKQQTEAIDALNKASSENTQNIAKNQADIANNINNIYELAQQQDQHSSDIKT |
| | LAKVSAANTDRIAKNKADADASFETLTKNQNTLIEKDKEHDKLITANKTAIDANKASADTKF |
| | AATADAITKNGNAITKNAKSITDLGTKVDGFDGRVTALDTKVNAFDGRITALDSKVENGMA |
| | AQAALSGLFQPYSVGKFNATAALGGYGSKSAVAIGAGYRVNPNLAFKAGAAINTSGNKK |
| | GSYNIGVNYEF (684 amino acids) |
| Dutch H2 | MKTMKLLPLKIAVTSAMMVGLGMASTANAQQQKSPKTEIFLPNLFDNDNTELTDPLYHNM |
| (SEQ ID | ILGNTALLTQENQYKFYADDGNGVPDSLLFNKILHDQLLHGFKKGDTIIPLDENGKPVYKL |
| NO: 9) | DSIVEQGKTKTVYSVTTKTATADDVNSAYSRGIQGDIDDLYEANKENVNRLIEHGDKIFAN |
| | EESVQYLNREVQNNIENIYELVQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGAAINTSG |
| | NKKGSYNIGVNYEF (684 amino acids) |
| Dutch F10 | MKTMKLLPLKIAVTSAMIIGLGAASTANAQLAEQFFPNIFSNHAPVKQHYHNVVVGDTSIV |
| (SEQ ID | ENLQDSDDTQLKFYSNDEYSVPDSLLFNKMLHEQQLNGFKKGDTIIPLDENGKPVYKVDY |
| NO: 10) | KLDGQEPRRVYSVTTKIATQDDVDNSPYSRGIQGDIDDLYEANKENVNRLIEHGDKIFANE |
| | ESVQYLNKEVQNNIENIYELAQQQDQHSSDIKTLKKNVEEGLLELSGHLIDQKADLTKDIKT |
| | LESNVEEGLLELSGHLIDQKADIAKNQADIAQNQANIQDLAAYNELQDAYAKQQTEAIDAL |
| | NKASSENTQNIEDLAAYNELQDAYAKQQTEAIDALNKASSENTQNIAKNQADIANNINNIY |
| | ELAQQQDQHSSDIKTLAKASAANTDRIAKNKADADASFETLTKNQNTLIEKDKEHDKLITA |
| | NKTAIDANKASADTKFAATADAITKNGNAITKNAKSITDLGTKVDAFDGRVTALDTKVNAF |
| | DGRITALDSKVENGMAAQAALSGLFQPYSVGKFNATAALGGYGSKSAVAIGAGYRVNPN |
| | LAFKAGAAINTSGNKKGSYNIGVNYEF (574 amino acids) |
| Norwegian | MKTMKLLPLKIAVTSALIVGLGAASTANAQQQPQTETFFPNIFFNENHDALDDVYHNMILG |
| 1 | DTAITQDNQYKFYADAISEVPDSLLFNKILHDQQLNGFKEGDTIIPLDENGKPVYKLDEKVE |
| (SEQ ID | NGVKKSVYSVTTKTATRADVEQSAYSRGIQGDIDDLYEANKENVNRLIEHGDKIFANEES |
| NO: 11) | VQYLNKEVQNNIENIHELAQQQDQHSSDIKTLKKNVEEGLLELSGHLIDQKADLTKDIKTLE |
| | SNVEEGLLDLSGRLLDQKADIAQNQANIQDLAAYNELQDAYAKQQTEAIDALNKASSENT |
| | QNIEDLAAYNELQDAYAKQQTEAIDALNKASSENTQNIEDLAAYNELQDAYAKQQTEAIDA |
| | LNKASSENTQNIEDLAAYNELQDAYAKQQTEAIDALNKASSENTQNIEDLAAYNELQDAY |
| | AKQQTEAIDALNKASSENTQNIAKNQADIANNINNIYELAQQQDQHSSDIKTLAKASAANT |
| | DRIAKNKADADASFETLTKNQNTLIEKDKEHDKLITANKTAIDANKASADTKFAATADAITK |
| | NGNAITKNAKSITDLGTKVDAFDGRVTALDTKVNALDTKVNAFDGRITALDSKVENGMAA |
| | QAALSGLFQPYSVGKFNATAALGGYGSKSAVAIGAGYRVNPNLAFKAGAAINTSGNKKG |
| | SYNIGVNYEF (678 amino acids) |
| Norwegian | MKTMKLLPLKIAVTSAMIVGLGAASTANAQQQQQPRTETFFPNIFFNENHDALDDVYHNM |
| 13 | ILGDTAITQDNQYKFYADAISEVPDSLLFNKILHDQQLNGFKEGDTIIPLDENGKPVYKLDE |
| (SEQ ID | KVENGVKKSVYSVTTKTATRADVEQSAYSRGIQGDIDDLYEANKENVNRLIEHGDKIFAN |
| NO: 12) | EESVQYLNREVQNNIENIHELAQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKTLENNIEEGLLELSGHLIDQKADLTKDIKALESNVEEGLLDLSGRLLDQK |
| | ADIAQNQANIQDLAAYNELQDQYAQKQTEAIDALNKASSENTQNIEDLAAYNELQDAYAK |
| | QQTEAIDALNKASSENTQNIAKNQADIANNINNIYELAQQQDQHSSDIKTLAKASAANTDRI |
| | AKNKADADASFETLTKNQNTLIEKDKEHDKLITANKTAIDTNKASADTKFAATADAITKNGN |
| | AITKNAKSITDLGTKVDGFDGRVTALDTKVNALDTKVNAFDGRITALDSKVENGMAAQAAL |
| | SGLFQPYSVGKFNATAALGGYGSKSAVAIGAGYRVNPNLAFKAGAAINTSGNKKGSYNIG |
| | VNYEF (678 amino acids) |
| Norwegian | MKTMKLLPLKIAVTSALIVGLGAASTANAQLVERFFPNIFLDKPLAKQHYHNVVVGDTSIVS |
| 20 | DLQSNSDQLKFYSDDEGLVPDSLLFNKMLHEQLLNGFKEGDTIIPLDENGKPVYKVDYKL |
| (SEQ ID | DGKEPRKVYSVTTKIATAEDVATSSYANGIQKDIDDLYDFDHQVTERLTQHGKTIYRNGE |
| NO: 13) | RILANEESVQYLNKEVQNNIEHIYELAQQQDQHSSDIKTLESNVEKGLLELSGHLIDQKAD |
| | LTKDIKTLENNVEEGLLDLSGRLIDQKADIAQNQANIQDLAAYNELQDQYAQKQTEAIDAL |
| | NKASSENTQNIEDLAAYNELQDAYAKQQTEAIDALNKASSENTQNIAKNQADIANNINNIY |
| | ELAQQQDQHSSDIKTLAKASAANTDRIAKNKADADASFETLTKNQNTLIEKDKEHDKLITA |
| | NKTAIDTNKASADTKFAATADAITKNGNAITKNAKSITDLGTKVDGFDSRVTALDTKVNALD |
| | TKVNALDTKVNAFDGRITALDSKVENGMAAQAALSGLFQPYSVGKFNATAALGGYGSKS |
| | AVAIGAGYRVNPNLAFKAGAAINTSGNKKGSYNIGVNYEF (587 amino acids) |
| Norwegian | MKTMKLLPLKIAVTSAMIVGLGAASTANAQQQQQPRTETFFPNIFFNENHDALDDVYHNM |
| 25 | ILGDTAITQDNQYKFYADAISEVPDSLLFNKILHDQQLNGFKEGDTIIPLDENGKPVYKLDE |
| (SEQ ID | KVENGVKKSVYSVTTKTATRADVEQSAYSRGIQGDIDDLYEANKENVNRLIEHGDKIFAN |
| NO: 14) | EESVQYLNREVQNNIENIHELAQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKTLENNIEEGLLELSGHLIDQKADLTKDI KALESNVEEGLLDLSGRLLDQK |
| | ADIAQNQANIQDLAAYNELQDQYAQKQTEAIDALNKASSENTQNIEDLAAYNELQDAYAK |
| | QQTEAIDALNKASSENTQNIAKNQADIANNINNIYELAQQQDQHSSDIKTLAKASAANTDRI |
| | AKNKADADASFETLTKNQNTLIEKDKEHDKLITANKTAIDTNKASADTKFAATADAITKNGN |
| | AITKNAKSITDLGTKVDGFDGRVTALDTKVNALDTKVNAFDGRITALDSKVENGMAAQAAL |
| | SGLFQPYSVGKFNATAALGGYGSKSAVAIGAGYRVNPNLAFKAGAAINTSGNKKGSYNIG |
| | VNYEF (678 amino acids) |
| Norwegian | MKTMKLLPLKIAVTSALIVGLGAASTANAQVRDKSLEDIEALLGKIDISKLEKEKKQQTELQ |
| 27 | KYLLLSQYANVLTMEELNKNVEKNTNSIEALGYEIGWLENDIADLEEGVEELTKNQNTLIEK |
| (SEQ ID | DEEHDRLIAQNQADIKTLENNVVEELFNLSDRLIDQEADIAKNNASIEELYDFDNEVAERIG |
| NO: 15) | EIHAYTEEVNKTLEKLITNSVKNTDNIDKNKADIQALENNVEEGLLELSGHLIDQKADLTKDI |
| | KALESNVEEGLLDLSGRLLDQKADIAKNQADIAQNQTDIQDLAAYNELQDQYAQKQTEAI |
| | DALNKASSENTQNIEDLAAYNELQDAYAKQQTEAIDALNKASSENTQNIEDLAAYNELQD |
| | AYAKQQTEAIDALNKASSENTQNIAKNQADIANNINNIYELAQQQDQHSSDIKTLAKVSAA |
| | NTDRIAKNKADADASFETLTKNQNTLIEKDKEHDKLITANKTAIDANKASADTKFAATADAI |
| | TKNGNAITKNAKSITDLGTKVDGFDSRVTALDTKVNAFDGRITALDSKVENGMAAQAALS |
| | GLFQPYSVGKFNATAALGGYGSKSAVAIGAGYRVNPNLAFKAGAAINTSGNKKGSYNIGV |
| | NYEF (616 amino acids) |
| Norwegian | MKTMKLLPLKIAVTSALIVGLGAASTANAQATETFLPNLFDNDYTETTDPLYHGMILGNTAI |
| 36 | TQDTQYKFYAENGNEVPDSLFFNKILHDQQLNGFKEGDTIIPLDENGKPVYKLDEITENGV |
| (SEQ ID | KRKVYSVTTKTATREDVEQSAYSRGIQGDIDDLYEANKENVNRLIEHGDKIFANEESVQYL |
| NO: 16) | NKEVQNNIENIHELAQQQDQHSSDIKTLKKNVEEGLLELSGHLIDQKADLTKDIKALESNV |
| | EEGLLDLSGHLIDQKADLTKDIKALESNVEEGLLDLSGRLLDQKADIAKNQADIAQNQTDIQ |
| | DLAAYNELQDQYAQKQTEAIDALNKASSENTQNIEDLAAYNELQDQYAQKQTEAIDALNK |
| | ASSENTQNIEDLAAYNELQDQYAQKQTEAIDALNKASSENTQNIEDLAAYNELQDQYAQK |
| | QTEAIDALNKASSENTQNIAKNQADIANNINNIYELAQQQDQHSSDIKTLAKASAANTDRIA |
| | KNKADADASFETLTKNQNTLIEKDKEHDKLITANKTAIDANKASADTKFAATADAITKNGNA |
| | ITKNAKSITDLGTKVDGFDGRVTALDTKVNALDTKVNAFDGRITALDSKVENGMAAQAALS |
| | GLFQPYSVGKFNATAALGGYGSKSAVAIGAGYRVNPNLAFKAGAAINTSGNKKGSYNIGV |
| | NYEF (676 amino acids) |
| BC5SV | MKTMKLLPLKIAVTSALIVGLGAASTANAQNGTSTKLKNLKEYAQYLDNYAQYLDDDIDDL |
| (SEQ ID | DKEVGELSQNIAKNQANIKDLNKKLSRDIDSLREDVYDNQYEIVNNQADIEKNQDDIKELE |
| NO: 17) | NNVGKELLNLSGRLLDQKADIDNNINNIYELAQQQDQHSSDIKTLKKNVEEGLLELSGHLI |
| | DQKSDIAQNQTDIQDLATYNELQDQYAQKQTEAIDALNKASSENTQNIEDLAAYNELQDA |
| | YAKQQTEAIDALNKASSENTQNIQDLAAYNELQDAYAKQQTEAIDALNKASSENTQNIEDL |
| | AAYNELQDAYAKQQTEAIDALNKASSENTQNIEDLAAYNELQDAYAKQQTEAIDALNKAS |
| | SENTQNIEDLAAYNELQDAYAKQQTEAIDALNKASSENTQNIAKNQADIANNINNIYELAQ |
| | QQDQHSSDIKTLAKASAANTDRIAKNKADADASFETLTKNQNTLIEKDKEHDKLITANKTAI |
| | DANKASADTKFAATADAITKNGNAITKNAKSITDLGTKVDAFDGRVTALDTKVNAFDGRIT |
| | ALDSKVENGMAAQAALSGLFQPYSVGKFNATAALGGYGSKSAVAIGAGYRVNPNLAFKA |
| | GAAINTSGNKKGSYNIGVNYEF (629 amino acids) |
| Norwegian | MKTMKLLPLKIAVTSAMIVGLGMASTANAQQQRSPKTETFLPNIFFNEYADDLDTLYHNMI |
| 14 | LGDTAITHDDQYKFYADDATEVPDSLFFNKILHDQLLYGFKEGDKIIPLDENGKPVYKLDK |
| (SEQ ID | RLDNGVQKTVYSVTTKTATADDVNSAYSRGIQGDIDDLYEANKENVNRLIEHGDKIFANE |
| NO: 18) | ESVQYLNKEVQNNIENIHELAQQQDQHSSDIKTLKKNVEEGLLELSGHLIDQKTDIAQNQT |
| | DIQDLATYNELQDQYAQKQTEAIDALNKASSENTQNIAKNSNRIKALENNIEEGLLELSGHL |
| | IDQKADLTKDIKALESNVEEGLLDLSGRLIDQKADIAQNQANIQDLAAYNELQDAYAKQQT |
| | EAIDALNKASSENTQNIEDLAAYNELQDAYAKQQTEAIDALNKASSENTQNIEDLAAYNEL |
| | QDAYAKQQTEAIDALNKASSENTQNIAKNQADIANNINNIYELAQQQDQHSSDIKTLAKAS |
| | AANTDRIAKNKADADASFETLTKNQNTLIEKDKEHDKLITANKTAIDANKASADTKFAATAD |
| | AITKNGNAITKNAKSITDLGTKVDGFDGRVTALDTKVNALDTKVNAFDGRITALDSKVENG |
| | MAAQAALSGLFQPYSVGKFNATAALGGYGSKSAVAIGAGYRVNPNLAFKAGAAINTSGN |
| | KKGSYNIGVNYEF (683 amino acids) |
| Norwegian | MKTMKLLPLKIAVTSAMIVGLGAASTANAQAQSNRSLDQVQALLRGIDETKIKKEIQQSQQ |
| 3 | PELNKYLTFNQLANALNIEELNNNVQKNTQRLDSAATLYGDLSKTVPKSIKENKESIKENK |
| (SEQ ID | ESIKENKESIKENKESIKENKESIKENKESITTLTRKSFQNQVDIVRNNASIEDLYAYGQEVA |
| NO: 19) | KSIGEIHAYTEEVNKTLENLITNSVENTNNITKNKADIQALENNVVEELFNLSGRLIDQKADI |
| | DNNINNIYELAQQQDQHSSDIKTLKKNVEEGLLELSGHLIDQKADLTKDIKTLESNVEEGLL |
| | DLSGRLLDQKADIAQNQANIQDLAAYNELQDAYAKQQTEAIDALNKASSENTQNIEDLAAY |
| | NELQDAYAKQQTEAIDALNKASSENTQNIEDLAAYNELQDAYAKQQTEAIDALNKASSEN |
| | TQNIEDLAAYNELQDAYAKQQTEAIDALNKASSENTQNIAKNQADIANNINNIYELAQQQD |
| | QHSSDIKTLAKASAANTDRIAKNKADADASFETLTKNQNTLIEKDKEHDKLITANKTVIDAN |
| | KASADTKFAATADAITKNGNAITKNAKSITDLGTKVDGFDGRVTALDTKVNALDTKVNAFD |
| | GRITALDSKVENGMAAQAALSGLFQPYSVGKFNATAALGGYGSKSAVAIGAGYRVNPNL |
| | AFKAGAAINTSGNKKGSYNIGVNYEF (700 amino acids) |
| Finnish | MKTMKLLPLKIAVTSALIVGLGAASTANAQATETFLPNLFDNDYIETTDPLYHGMILGNTAIT |
| 414 | QDTQYKFYAENGNEVPDSLFFNKILHDQQLNGFKEGDTIIPLDENGKPVYKLDEITENGVK |
| (SEQ ID | RKVYSVTTKTATREDVEQSAYSRGIQGDIDDLYEANKENVNRLIEHGDKIFANEESVQYLN |
| NO: 20) | KEVQNNIENIHELAQQQDQHSSDIKTLKKNVEEGLLELSGHLIDQKADLTKDIKTLENNVEE |
| | GLLELSGHLIDQKADLTKDIKALESNVEEGLLDLSGRLLDQKADIAKNQADIAQNQTDIQDL |
| | AAYNELQDQYAQKQTEAIDALNKASSENTQNIEDLAAYNELQDQYAQKQTEAIDALNKAS |
| | SENTQNIEDLAAYNELQDQYAQKQTEAIDALNKASSENTQNIEDLAAYNELQDQYAQKQT |
| | EAIDALNKASSENTQNIAKNQADIANNINNIYELAQQQDQHSSDIKTLAKASAANTDRIAKN |
| | KADADASFETLTKNQNTLIEKDKEHDKLITANKTAIDANKASADTKFAATADAITKNGNAIT |
| | KNAKSITDLGTKVDGFDGRVTALDTKVNALDTKVNAFDGRITALDSKVENGMAAQAALSG |
| | LFQPYSVGKFNATAALGGYGSKSAVAIGAGYRVNPNLAFKAGAAINTSGNKKGSYNIGVN |
| | YEF (676 amino acids) |
| Japanese | MKTMKLLPLKIAVTSAMIIGLGAASTANAQLAEQFFPNIFSNHAPVKQHYHNVVVGDTSIV |
| Z7476 | ENLQDSDDTQLKFYSNDEYSVPDSLLFNKMLHEQQLNGFKKGDTIIPLDENGKPVYKVDY |
| (SEQ ID | KLDGQEPRRVYSVTTKIATQDDVDNSPYSRGIQGDIDDLYEANKENVNRLIEHGDKIFANE |
| NO: 21) | ESVQYLNKEVQNNIENIYELAQQQDQHSSDIKTLKKNVEEGLLELSGRLIDQKADIAQNQA |
| | NIQDLAAYNELQDQYAQKQTEAIDALNKASSENTQNIEDLAAYNELQDAYAKQQTEAIDAL |
| | NKASSENTQNIEDLAAYNELQDAYAKQQTEAIDALNKASSENTQNIEDLAAYNELQDAYA |
| | KQQTEAIDALNKASSENTQNIEDLAAYNELQDAYAKQQTEAIDALNKASSENTQNIEDLAA |
| | YNELQDAYAKQQTEAIDALNKASSENTQNIAKNQADIANNINNIYELAQQQDQHSSDIKTL |
| | AKVSAANTDRIAKNKADADASFETLTKNQNTLIEKDKEHDKLITANKTAIDANKASADTKFA |
| | ATADAITKNGNAITKNAKSITDLGTKVDGFDGRVTALDTKVNAFDGRITALDSKVENGMAA |
| | QAALSGLFQPYSVGKFNATAALGGYGSKSAVAIGAGYRVNPNLAFKAGAAINTSGNKKG |
| | SYNIGVNYEF (678 amino acids) |
| Belgian | MKTMKLLPLKIAVTSAMIIGLGAASTANAQSRDRSLEDIQDSISKLVQDDINTLKQDQQKM |
| Z7530 | NKYLLLNQLANTLITDELNNNVIKNTNSIEALGDEIGWLENDIADLEEGVEELTKNQNTLIEK |
| (SEQ ID | DEEHDRLIAQNQADIQTLENNVVEELFNLSGRLIDQEADIAKNNASIEELYDFDNEVAERIG |
| NO: 22) | EIHAYTEEVNKTLENLITNSVKNTDNIDKNKADIDNNINHIYELAQQQDQHSSDIKTLKNNV |
| | EEGLLELSGHLIDQKADLTKDIKALESNVEEGLLDLSGRLLDQKADLTKDIKALESNVEEGL |
| | LDLSGRLLDQKADIAQNQTDIQDLAAYNELQDQYAQKQTEAIDALNKASSENTQNIEDLAA |
| | YNELQDAYAKQQTEAIDALNKASSENTQNIAKNQADIANNINNIYELAQQQDQHSSDIKTL |
| | AKASAANTNRIATAELGIAENKKDAQIAKAQANANKTAIDENKASADTKFAATADAITKNG |
| | NAITKNAKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNIGVNYEF |
| | (613 amino acids) |
| German | MKTMKLLPLKIAVTSALIVGLGAASTANAQATNKDITLEDVLKSIEEIDPYELRDYIEYPTAIE |
| Z8063 | RFLLLSQYGNTLTLEEFDNDIELLDQDVEDLEESVTELAKNQNSLIEQGEAIKEDLQGLAD |
| (SEQ ID | FVERQEDKILQNETSIKKNTQRNLVNGFEIEKNKDAIAKNNESIEDLYDFGHEVAKSIGEIH |
| NO: 23) | AHNEAQNETLKDLITNSVKNTDNITKNKADIQALESNVEKGLLELSGHLIDQKADIDNNINNI |
| | HELAQQQDQHSSDIKTLKKNVEEGLLELSGHLIDQKSDIAQNQANIQDLATYNELQDQYA |
| | QKQTEAIDALNKASSENTQNIEDLAAYNELQDAYAKQQTEAIDALNKASSENTQNIAKNQA |
| | DIANNINNIYELAQQQDQHSSDIKTLAKASAANTDRIAKNKADADASFETLTKNQNTLIEKD |
| | KEHDKLITANKTAIDANKASADTKFAATADAITKNGNAITKNAKSITDLGTKVDGFDSRVTA |
| | LDTKVNAFDGRITALDSKVENGMAAQAALSGLFQPYSVGKFNATAALGGYGSKSAVAIGA |
| | GYRVNPNLAFKAGAAINTSGNKKGSYNIGVNYEF (589 amino acids) |
| American | MKTMKLLPLKIAVTSAMMVGLGMASTANAQQQKSPKTEIFLPNLFDNDNTELTDPLYHNM |
| O12E | ILGNTALLTQENQYKFYADDGNGVPDSLLFNKILHDQLLHGFKEGDTIIPLDENGKPVYKL |
| (SEQ ID | DSIVEQGKTKTVYSVTTKTATADDVNSAYSRGIQGDIDDLYEANKENVNRLIEHGDKIFAN |
| NO: 24) | EESVQYLNREVQNNIENIHELAQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGAAINTSG |
| | NKKGSYNIGVNYEF (684 amino acids) |
| Greek | MKTMKLLPLKIAVTSALIVGLGAASTANAQQQQKTKTEVFLPNLFYNDYIEETDLLYHNMIL |
| MC317 | GDTAALVDRQNYSNSQLKFYSNDEESVPDSLLFSKMLNNQQLNGFKAGDIIIPVDANGQV |
| (SEQ ID | IYQKDTRVEGGKTRTVLSVTTKIATQQDVDSAYSRGIQGKVNDLDDEMNFLNHDITSLYD |
| NO: 25) | VTANQQDDIKGLKKGVKDLKKGVKGLNKELKELDKEVGVLSRDIGSLNDDVAQNNESIED |
| | LYDFSQEVADSIGEIHAHNKAQNETLQDLITNSVENTNNITKNKADIQALENNVVEELFNLS |
| | GRLIDQKADLTKDIKTLESNVEEGLLELSGHLIDQKADIAKNQADIAQNQANIQDLAAYNEL |
| | QDAYAKQQTEAIDALNKASSENTQNIEDLAAYNELQDAYAKQQTEAIDALNKASSENTQNI |
| | AKNQADIANNINNIYELAQQQDQHSSDIKTLAKASAANTDRIAKNKADADASFETLTKNQN |
| | TLIEKDKEHDKLITANKTAIDENKASADTKFAATADAITKNGNAITKNAKSITDLGTKVDGFD |
| | GRVTALDTKVNAFDGRITALDSKVENGMAAQAALSGLFQPYSVGKFNATAALGGYGSKS |
| | AVAIGAGYRVNPNLAFKAGAAINTSGNKKGSYNIGVNYEF (650 amino acids) |
| American | MKTMKLLPLKIAVTSALIVGLGAVSTTNAQAQSRSLDQIQTKLADLAGKIAAGKNGGGQNN |
| V1122 | QNNQNDINKYLFLSQYANILTMEELNNNVVKNSSSIETLETDFGWLENDVADLEDGVEEL |
| (SEQ ID | TKNQNTLIEKDEEHDRLIAQNQADIQTLENNVVEELFNLSDRLIDQKADIAKNQADIAQNNE |
| NO: 26) | SIEELYDFDNEVAEKIGEIHAYTEEVNKTLQDLITNSVKNTDNIDKNKADIDNNINHIYELAQ |
| | QQDQHSSDIKTLKNNVEEGLLELSGHLIDQKADLTKDIKTLENNVEEGLLDLSGRLIDQKA |
| | DIAKNQADIAQNQTDIQDLAAYNELQDQYAQKQTEAIDALNKASSENTQNIEDLAAYNELQ |
| | DAYAKQQTEAIDALNKASSENTQNIAKNQADIANNINNIYELAQQQDQHSSDIKTLAKASA |
| | ANTDRIAKNKADADASFETLTKNQNTLIEKDKEHDKLITANKTAIDENKASADTKFAATADA |
| | ITKNGNAITKNAKSITDLGTKVDGFDGRVTALDTKVNAFDGRITALDSKVENGMAAQAALS |
| | GLFQPYSVGKFNATAALGGYGSKSAVAIGAGYRVNPNLAFKAGAAINTSGNKKGSYNIGV |
| | NYEF (616 amino acids) |
| American | MKTMKLLPLKIAVTSALIVGLGTASTANAQVASPANQKIQQKIKKVRKELRQDIKSLRNDID |
| P44 | SNTADIGSLNDDVADNQDDILDNQADIAKNQDDIEKNQADIKELDKEVGVLSREIGSLNDDI |
| (SEQ ID | ADNYTDIIDNYTDIIDNQANIAKNQDDIEKNQADIKELDKEVGVLSREIGSLNDDVADNQDDI |
| NO: 27) | AKNQADIQTLENNVEEGLLELSGHLLDQKADIDNNINNIYELAQQQDQHSSDIKTLKKNVE |
| | EGLLELSGHLIDQKTDIAQNQANIQDLATYNELQDQYAQEQTEAIDALNKASSENTQNIAK |
| | NSNRIKALESNVEEGLLELSGHLIDQKADLTKDIKALESNVEEGLLELSGHLIDQKADIAQN |
| | QANIQDLAAYNELQDQYAQKQTEAIDALNKASSENTQNIEDLAAYNELQDAYAKQQTEAI |
| | DALNKASSENTQNIAKNQADIANNINNIYELAQQQDQHSSDIKTLAKASAANTDRIAKNKA |
| | DADASFETLTKNQNTLIEKDKEHDKLITANKTAIDANKVSADTKFAATADAITKNGNAITKN |
| | AKSITDLGTKVDAFDSRVTALDTKVNAFDGRITALDSKVENGMAAQAALSGLFQPYSVGK |
| | FNATAALGGYGSKSAVAIGAGYRVNPNLAFKAGAAINTSGNKKGSYNIGVNYEF |
| | (668 amino acids) |
| American | MKTMKLLPLKIAVTSAMIVGLGATSTVNAQVVEQFFPNIFFNENHDELDDAYHNMILGDTA |
| V1171 | IVSNSQDNSTQLKFYSNDEDSVPDSLLFSKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQ |
| NO: 28) | TEAIDALNKASSANTDRIDTAEERIDKNEYDIKALESNVEEGLLELSGHLIDQKADLTKDIKA |
| | LESNVEEGLLELSGHLIDQKADLTKDIKALESNVEEGLLDLSGRLIDQKADIAQNQANIQDL |
| | AAYNELQDAYAKQQTEAIDALNKASSENTQNIEDLAAYNELQDAYAKQQTEAIDALNKAS |
| | SENTQNIEDLAAYNELQDAYAKQQTEAIDALNKASSENTQNIEDLAAYNELQDAYAKQQT |
| | EAIDALNKASSENTQNIAKNQADIANNINNIYELAQQQDQHSSDIKTLAKASAANTDRIAKN |
| | KADADASFETLTKNQNTLIEKDKEHDKLITANKTAIDANKASADTKFAATADAITKNGNAIT |
| | KNAKSITDLGTKVDGFDGRVTALDTKVNALDTKVNAFDGRITALDSKVENGMAAQAALSG |
| | LFQPYSVGKFNATAALGGYGSKSAVAIGAGYRVNPNLAFKAGAAINTSGNKKGSYNIGVN |
| | YEF (674 amino acids) |
| American | MKTMKLLPLKIAVTSAMIIGLGAASTANAQSRDRSLEDIQDSISKLVQDDIDTLKQDQQKM |
| TTA24 | NKYLLLNQLANTLITDELNNNVIKNTNSIEALGDEIGWLENDIADLEEGVEELTKNQNTLIEK |
| (SEQ ID | DEEHDRLIAQNQADIQTLENNVVEELFNLSGRLIDQEADIAKNNASIEELYDFDNEVAERIG |
| NO: 29) | EIHAYTEEVNKTLENLITNSVKNTDNIDKNKADIDNNINHIYELAQQQDQHSSDIKTLKNNV |
| | EEGLLELSGHLIDQKADLTKDIKALESNVEEGLLDLSGRLLDQKADLTKDIKALESNVEEGL |
| | LDLSGRLLDQKADIAQNQTDIQDLAAYNELQDQYAQKQTEAIDALNKASSENTQNIEDLAA |
| | YNELQDAYAKQQTEAIDALNKASSENTQNIAKNQADIANNINNIYELAQQQDQHSSDIKTL |
| | AKASAANTNRIATAELGIAENKKDAQIAKAQANANKTAIDENKASADTKFAATADAITKNG |
| | NAITKNAKSITDLGTKVDGFDGRVTALDTKVNAFDGRITALDSKVENGMAAQAALSGLFQ |
| | PYSVGKFNATAALGGYGSKSAVAIGAGYRVNPNLAFKAGAAINTSGNKKGSYNIGVNYEF |
| | (613 amino acids) |
| American | MKTMKLLPLKIAVTSAMIVGLGATSTVNAQVVEQFFPNIFFNENHDELDDAYHNMILGDTA |
| O35E | IVSNSQDNSTQLKFYSNDEDSVPDSLLFSKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQ |
| NO: 30) | TEAIDALNKASSANTDRIDTAEERIDKNEYDIKALESNVEEGLLELSGHLIDQKADLTKDIKA |
| | LESNVEEGLLELSGHLIDQKADLTKDIKALESNVEEGLLDLSGRLLDQKADIAKNQADIAQN |
| | QTDIQDLAAYNELQDAYAKQQTEAIDALNKASSENTQNIAKNQADIANNINNIYELAQQQD |
| | QHSSDIKTLAKASAANTDRIAKNKADADASFETLTKNQNTLIEKDKEHDKLITANKTAIDAN |
| | KASADTKFAATADAITKNGNAITKNAKSITDLGTKVDGFDGRVTALDTKVNALDTKVNAFD |
| | GRITALDSKVENGMAAQAALSGLFQPYSVGKFNATAALGGYGSKSAVAIGAGYRVNPNL |
| | AFKAGAAINTSGNKKGSYNIGVNYEF (576 amino acids) |
| American | MKTMKLLPLKIAVTSAMMVGLGMASTANAQQQKSPKTEIFLPNLFDNDNTELTDPLYHNM |
| SP12-6 | ILGNTALLTQENQYKFYADDGNGVPDSLLFNKILHDQLLHGFKEGDTIIPLDENGKPVYKL |
| (SEQ ID | DSIVEQGKTKTVYSVTTKTATADDVNSAYSRGIQGDIDDLYEANKENVNRLIEHGDKIFAN |
| NO: 31) | EESVQYLNREVQNNIENIHELAQQQDQHSSDIKTLKKNVEKDLLDLSGRLIAQKEDIAQNQ |
| | TDIQDLATYNELQDQYAQKQTEAIDALNKASSENTQNIAKNSNHIKTLENNIEEGLLELSGH |
| | LIDQKADLTKDIKALESNVEEGLLDLSGRLIDQKADIAQNQANIQDLAAYNELQDAYAKQQ |
| | TEAIDALNKASSENTQNIEDLAAYNELQDAYAKQQTEAIDALNKASSENTQNIEDLAAYNE |
| | LQDAYAKQQTEAIDALNKASSENTQNIAKNQADIANNINNIYELAQQQDQHSSDIKTLAKA |
| | SAANTDRIAKNKADADASFETLTKNQNTLIEKDKEHDKLITANKTAIDANKASADTKFAATA |
| | DAITKNGNAITKNAKSITDLGTKVDGFDGRVTALDTKVNALDTKVNAFDGRITALDSKVEN |
| | GMAAQAALSGLFQPYSVGKFNATAALGGYGSKSAVAIGAGYRVNPNLAFKAGAAINTSG |
| | NKKGSYNIGVNYEF (684 amino acids) |
| American | MKTMKLLPLKIAVTSAMIIGLGAASTANAQATETFLPNLFDNDYTETTDPLYHGMILGNTAI |
| SP12-5 | TQDTQYKFYAENGNEVPDSLFFNKILHDQQLNGFKEGDTIIPLDENGKPVYKLDEITENGV |
| (SEQ ID | KRKVYSVTTKTATREDVEQSAYSRGIQGDIDDLYEANKENVNRLIEHGDKIFANEESVQYL |
| NO: 32) | NKEVQNNIENIHELAQQQDQHSSDIKTLKKNVEEGLLELSGRLIAQKEDIAQNQTDIQDLA |
| | TYNELQDQYAQKQTEAIDALNKASSENTQNIAKNSNHIKTLENNIEEGLLELSGHLIDQKA |
| | DLTKDIKALESNVEEGLLDLSGRLLDQKADIAKNQADIAQNQTDIQDLAAYNELQDQYAQK |
| | QTEAIDALNKASSENTQNIEDLAAYNELQDAYAKQQTEAIDALNKASSENTQNIEDLAAYN |
| | ELQDAYAKQQTEAIDALNKASSENTQNIAKNQADIANNINNIYELAQQQDQHSSDIKTLAK |
| | ASAANTDRIAKNKADADASFETLTKNQNTLIEKDKEHDKLITANKTAIDANKASADTKFAAT |
| | ADAITKNGNAITKNAKSITDLGTKVDGFDGRVTALDTKVNALDTKVNAFDGRITALDSKVE |
| | NGMAAQAALSGLFQPYSVGKFNATAALGGYGSKSAVAIGAGYRVNPNLAFKAGAAINTS |
| | GNKKGSYNIGVNYEF (686 amino acids) |
| Swedish | MKTMKLLPLKIAVTSAMIIGLGAASTANAQAKNDITLEDLPYLIKKIDQNELEADIGDITALEK |
| BC5 | YLALSQYGNILALEELNKALEELDEDVGWNQNDIANLEDDVETLTKNQNALAEQGEAIKE |
| (SEQ ID | DLQGLADFVEGQEGKILQNETSIKKNTQRNLVNGFEIEKNKDAIAKNNESIEDLYDFGHEV |
| NO: 33) | AESIGEIHAHNEAQNETLKGLITNSIENTNNITKNKADIQALENNVVEELFNLSGRLIDQKAD |
| | IDNNINNIYELAQQQDQHSSDIKTLKKNVEEGLLELSGHLIDQKTDIAQNQANIQDLATYNE |
| | LQDQYAQKQTEAIDALNKASSENTQNIEDLAAYNELQDAYAKQQTEAIDALNKASSENTQ |
| | NIEDLAAYNELQDAYAKQQTEAIDALNKASSENTQNIAKNQADIANNINNIYELAQQQDQH |
| | SSDIKTLAKASAANTDRIAKNKADADASFETLTKNQNTLIEKDKEHDKLITANKTAIDANKA |
| | SADTKFAATADAITKNGNAITKNAKSITDLGTKVDGFDSRVTALDTKVNAFDGRITALDSKV |
| | ENGMAAQAALSGLFQPYSVGKFNATAALGGYGSKSAVAIGAGYRVNPNLAFKAGAAINT |
| | SGNKKGSYNIGVNYEF (630 amino acids) |
| American | MKTMKLLPLKIAVTSALIVGLGAASTANAQAQDRSLEQIQDKLANLVEKIEQAKSQNGQSQ |
| 7169 | KDINQYLLLSQYANVLTMEELNNNVVKNSSSIETLDNDIAWLNDDLIDLDKEVGVLSRDIGS |
| (SEQ ID | LHDDVAQNQADIKTLKNNVVEELFNLSDRLIDQEADIAQNNESIEDLYDFGREVAESIGEIH |
| NO: 34) | AHNEAQNETLKDLITNSVKNTDNITKNKADIQALENDVGKELLNLSGRLIDQKADIDNNINHI |
| | YELAQQQDQHSSDIKTLKNNVEEGLLELSGHLIDQKADLTKDIKALESNVEEGLLDLSGRL |
| | LDQKADIAQNQANIQDLAAYNELQDAYAKQQTEAIDALNKASSENTQNIEDLAAYNELQD |
| | AYAKQQTEAIDALNKASSENTQNIAKNQADIANNINNIYELAQQQDQHSSDIKTLAKASAA |
| | NTDRIAKNKADADASFETLTKNQNTLIEKDKEHDKLITANKTAIDANKASADTKFAATADAI |
| | TKNGNAITKNAKSITDLGTKVDGFDSRVTALDTKVNAFDGRITALDSKVENGMAAQAALS |
| | GLFQPYSVGKFNATAALGGYGSKSAVAIGAGYRVNPNLAFKAGAAINTSGNKKGSYNIGV |
| | NYEF (616 amino acids) |
| Finnish | MKTMKLLPLKIAVTSAMIIGLGATSTVNAQVVEQFFPNIFFNENHDELDDAYHNMILGDTAI |
| FIN2344 | VSNSQDNSTQLKFYSNDEDSVPDSLLFSKLLHEQQLNGFKAGDTIIPLDKDGKPVYTKDT |
| (SEQ ID | RTKDGKVETVYSVTTKIATQDDVEQSAYSRGIQGDIDDLYDINREVNEYLKATHDYNERQ |
| NO: 35) | TEAIDALNKASSANTDRIDTAEERIDKNEYDIKALESNVGKDLLDLSGRLIAQKEDIDNNINH |
| | IYELAQQQDQHSSDIKTLKNNVEEGLLELSGHLIDQKADLTKDIKTLESNVEEGLLDLSGRL |
| | IDQKADIAQNQANIQDLAAYNELQDQYAQKQTEAIDALNKASSENTQNIEDLAAYNELQDA |
| | YAKQQTEAIDALNKASSENTQNIAKNQADIANNINNIYELAQQQDQHSSDIKTLAKVSAAN |
| | TDRIAKNKADADASFETLTKNQNTLIEKDKEHDKLITANKTAIDANKASADTKFAATADAIT |
| | KNGNAITKNAKSITDLGTKVDGFDGRVTALDTKVNAFDGRITALDSKVENGMAAQAALSG |
| | LFQPYSVGKFNATAALGGYGSKSAVAIGAGYRVNPNLAFKAGAAINTSGNKKGSYNIGVN |
| | YEF (614 amino acids) |
| American | MKTMKLPPLKIAVTSAMIIGLGAASTANAQTTETFLPNLFDNDYTETTDPLYHGMILGDTAI |
| V1118 | TQDTQYKFYAENGNEVPDSLFFNKILHDQLLNGFKAGDTIIPLDENGKPVYKLDERTENG |
| (SEQ ID | VKRKVYSVTTKTATQADVEQSAYSRGIQGDIDDLYEANKENVNRLIEHGDKIFANEESVQ |
| NO: 36) | YLNREVQNNIENIHELAQQQDQHSSDIKTLKKNVEKDLLDLSGRLIAQKEDIAQNQTDIQD |
| | LATYNELQDQYAQKQTEAIDALNKASSENTQNIAKNSNHIKTLENNIEECLLELSGHLIDQK |
| | ADLTKDIKALESNVEEGLLDLSGRLIDQKADIAQNQANIQDLAAYNELQDAYAKQQTEAID |
| | ALNKASSENTQNIEDLAAYNELQDAYAKQQTEAIDALNKASSENTQNIEDLAAYNELQDA |
| | YAKQQTEAIDALNKASSENTQNIAKNQADIANNINNIYELAQQQDQHSSDIKTLAKASAAN |
| | TDRIAKNKADADASFETLTKNQNTLIEKDKEHDKLITANKTAIDANKASADTKFAATADAIT |
| | KNGNAITKNAKSITDLGTKVDGFDGRVTALDTKVNALDTKVNAFDGRITALDSKVENGMA |
| | AQAALSGLFQPYSVGKFNATAALGGYGSKSAVAIGAGYRVNPNLAFKAGAAINTSGNKK |
| | GSYNIGVNYEF (679 amino acids) |
| American | MKTMKLLPLKIAVTSALIVGLGAASTANAQETLEEVLESIKQINEQDLQDDIGYNSALDRYL |
| V1145 | VLSQYGNLLIAKELNENVEKNSNSIAKNSNSIADLEADVGYLAENQNTLIEQNETINQELEG |
| (SEQ ID | ITHELESFIAYAHAQDQKNLVNEFEIEKNKDAIAKNNESIEDLYDFGHEVAESIGEIHAYTEE |
| NO: 37) | VNKTLENLITNSVKNTDNITKNKADIQALESNVEKELLNLSGRLIDQKADIDNNINHIYELAQ |
| | QQDQHSSDIKTLKKNVEEGLLELSGHLIDQKSDIAQNQTDIQDLATYNELQDQYAQKQTE |
| | AIDALNKASSENTQNIEDLAAYNELQDAYAKQQTEAIDALNKASSENTQNIEDLAAYNELQ |
| | DAYAKQQTEAIDALNKASSENTQNIEDLAAYNELQDAYAKQQTEAIDALNKASSENTQNIE |
| | DLAAYNELQDAYAKQQTEAIDALNKASSENTQNIEDLAAYNELQDAYAKQQTEAIDALNK |
| | ASSENTQNIAKNQADIANNINNIYELAQQQDQHSSDIKTLAKASAANTDRIAKNKADADAS |
| | FETLTKNQNTLIEKDKEHDKLITANKTAIDANKASADTKFAATADAITKNGNAITKNAKSITD |
| | LGTKVDGFDSRVTALDTKVNAFDGRITALDSKVENGMAAQAALSGLFQPYSVGKFNATA |
| | ALGGYGSKSAVAIGAGYRVNPNLAFKAGAAINTSGNKKGSYNIGVNYEF (724 amino |
| | acids) |
| American | MKTMKLLPLKIAVTSALIVGLGAASTANAQAQARDRSLEDIQALIGNIDVDKIRSQKQKNPE |
| V1156 | IFQYLLLNQLSNTLITDELNNNVIKNTNSIETLDNDIAWLNDDLIDLDKEVGVLSRDIGSLHD |
| (SEQ ID | DVAQNQADIKTLENNVVEELFNLSDRLIDQEAEIAQNNESIEDLYDFGREVAESIGEIHAHN |
| NO: 38) | EAQNETLKDLITNSVKNTDNIDKNKADIQALENNVEEGLLELSGHLIDQKADLTKDIKALES |
| | NVEEGLLDLSGRLLDQKADIAKNQADIAQNQTDIQDLAAYNELQDQYAQKQTEAIDALNK |
| | ASSENTQNIEDLAAYNELQDAYAKQQTEAIDALNKASSENTQNIEDLAAYNELQDAYAKQ |
| | QTEAIDALNKASSENTQNIAKNQADIANNINNIYELAQQQDQHSSDIKTLAKVSAANTDRIA |
| | KNKADADASFETLTKNQNTLIEKDKEHDKLITANKTAIDANKASADTKFAATADAITKNGNA |
| | ITKNAKSITDLGTKVDGFDSRVTALDTKVNAFDGRITALDSKVENGMAAQAALSGLFQPYS |
| | VGKFNATAALGGYGSKSAVAIGAGYRVNPNLAFKAGAAINTSGNKKGSYNIGVNYEF |
| | (611 amino acids) |

```
SEQ ID NO: 39
<210> 1
<211> 360
<212> PRT
<213> Haemophilus influenzae
<400> 1
Met Lys Leu Lys Thr Leu Ala Leu Ser Leu Leu Ala Ala Gly Val Leu
1 5 10 15
Ala Gly Cys Ser Ser His Ser Ser Asn Met Ala Asn Thr Gln Met Lys
                20 25 30
Ser Asp Lys Ile Ile Ile Ala His Arg Gly Ala Ser Gly Tyr Leu Pro
          35 40 45
Glu His Thr Leu Glu Ser Lys Ala Leu Ala Phe Ala Gln Gln Ala Asp
    50 55 60
Tyr Leu Glu Gln Asp Leu Ala Met Thr Lys Asp Gly Arg Leu Val Val
65 70 75 80
Ile His Asp His Phe Leu Asp Gly Leu Thr Asp Val Ala Lys Lys Phe
                    85 90 95
Pro His Arg His Arg Lys Asp Gly Arg Tyr Tyr Val Ile Asp Phe Thr
                100 105 110
Leu Lys Glu Ile Gln Ser Leu Glu Met Thr Glu Asn Phe Glu Thr Lys
          115 120 125
Asp Gly Lys Gln Ala Gln Val Tyr Pro Asn Arg Phe Pro Leu Trp Lys
    130 135 140
Ser His Phe Arg Ile His Thr Phe Glu Asp Glu Ile Glu Phe Ile Gln
145 150 155 160
Gly Leu Glu Lys Ser Thr Gly Lys Lys Val Gly Ile Tyr Pro Glu Ile
                  165 170 175
Lys Ala Pro Trp Phe His His Gln Asn Gly Lys Asp Ile Ala Ala Glu
                180 185 190
Thr Leu Lys Val Leu Lys Lys Tyr Gly Tyr Asp Lys Lys Thr Asp Met
          195 200 205
Val Tyr Leu Gln Thr Phe Asp Phe Asn Glu Leu Lys Arg Ile Lys Thr
    210 215 220
Glu Leu Leu Pro Gln Met Gly Met Asp Leu Lys Leu Val Gln Leu Ile
225 230 235 240
Ala Tyr Thr Asp Trp Lys Glu Thr Gln Glu Lys Asp Pro Lys Gly Tyr
                    245 250 255
Trp Val Asn Tyr Asn Tyr Asp Trp Met Phe Lys Pro Gly Ala Met Ala
                260 265 270
Glu Val Val Lys Tyr Ala Asp Gly Val Gly Pro Gly Trp Tyr Met Leu
          275 280 285
Val Asn Lys Glu Glu Ser Lys Pro Asp Asn Ile Val Tyr Thr Pro Leu
    290 295 300
Val Lys Glu Leu Ala Gln Tyr Asn Val Glu Val His Pro Tyr Thr Val
305 310 315 320
Arg Lys Asp Ala Leu Pro Glu Phe Phe Thr Asp Val Asn Gln Met Tyr
                  325 330 335
Asp Ala Leu Leu Asn Lys Ser Gly Ala Thr Gly Val Phe Thr Asp Phe
              340 345 350
Pro Asp Thr Gly Val Glu Phe Leu
          355 360
SEQ ID NO: 40
<210> 2
<211> 348
<212> PRT
<213> Artificial
<220>
<223> Protein D fragment with MDP tripeptide from NS1
<400> 2
Met Asp Pro Ser Ser His Ser Ser Asn Met Ala Asn Thr Gln Met Lys
1 5 10 15
Ser Asp Lys Ile Ile Ile Ala His Arg Gly Ala Ser Gly Tyr Leu Pro
              20 25 30
Glu His Thr Leu Glu Ser Lys Ala Leu Ala Phe Ala Gln Gln Ala Asp
          35 40 45
Tyr Leu Glu Gln Asp Leu Ala Met Thr Lys Asp Gly Arg Leu Val Val
    50 55 60
Ile His Asp His Phe Leu Asp Gly Leu Thr Asp Val Ala Lys Lys Phe
65 70 75 80
Pro His Arg His Arg Lys Asp Gly Arg Tyr Tyr Val Ile Asp Phe Thr
                    85 90 95
Leu Lys Glu Ile Gln Ser Leu Glu Met Thr Glu Asn Phe Glu Thr Lys
              100 105 110
Asp Gly Lys Gln Ala Gln Val Tyr Pro Asn Arg Phe Pro Leu Trp Lys
        115 120 125
Ser His Phe Arg Ile His Thr Phe Glu Asp Glu Ile Glu Phe Ile Gln
    130 135 140
Gly Leu Glu Lys Ser Thr Gly Lys Lys Val Gly Ile Tyr Pro Glu Ile
145 150 155 160
Lys Ala Pro Trp Phe His His Gln Asn Gly Lys Asp Ile Ala Ala Glu
                    165 170 175
Thr Leu Lys Val Leu Lys Lys Tyr Gly Tyr Asp Lys Lys Thr Asp Met
              180 185 190
Val Tyr Leu Gln Thr Phe Asp Phe Asn Glu Leu Lys Arg Ile Lys Thr
          195 200 205
Glu Leu Leu Pro Gln Met Gly Met Asp Leu Lys Leu Val Gln Leu Ile
    210 215 220
Ala Tyr Thr Asp Trp Lys Glu Thr Gln Glu Lys Asp Pro Lys Gly Tyr
225 230 235 240
Trp Val Asn Tyr Asn Tyr Asp Trp Met Phe Lys Pro Gly Ala Met Ala
                  245 250 255
Glu Val Val Lys Tyr Ala Asp Gly Val Gly Pro Gly Trp Tyr Met Leu
                260 265 270
Val Asn Lys Glu Glu Ser Lys Pro Asp Asn Ile Val Tyr Thr Pro Leu
          275 280 285
Val Lys Glu Leu Ala Gln Tyr Asn Val Glu Val His Pro Tyr Thr Val
    290 295 300
Arg Lys Asp Ala Leu Pro Glu Phe Phe Thr Asp Val Asn Gln Met Tyr
305 310 315 320
Asp Ala Leu Leu Asn Lys Ser Gly Ala Thr Gly Val Phe Thr Asp Phe
                  325 330 335
Pro Asp Thr Gly Val Glu Phe Leu Lys Gly Ile Lys
              340 345
SEQ ID NO: 41
<210> 3
<211> 989
<212> DNA
<213> Haemophilus influenzae
<400> 3
```

| | |
|---|---|
| gcgaataccc aaatgaaatc agacaaaatc attattgctc accgtggtgc tagcggttat | 60 |
| ttaccagagc atacgttaga atctaaagca cttgcgtttg cacaacacgc agattattta | 120 |
| gagcaagatt tagcaatgac taaggatggt cgtttagtgg ttattcacga tcacttttta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgactga aaactttgaa | 300 |
| accaaagacg gcaaacaagc gcaagtttat cctaatcgtt tcccactttg gaaatcacat | 360 |
| tttagaattc acacctttga agatgaaatt gagtttatcc aaggcttaga aaaatcgact | 420 |
| ggcagaaaag tagggattta tccagaaatc aaagcacctt ggttccacca tcaaaatggc | 480 |
| aaagatattg cagctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggaatgga tttaaaatta gttcaattaa ttgcttatac agattggaaa | 660 |
| gaaacacaag aaaaagaccc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactgccc | 900 |
| gagtttttca cagacgtaaa tcaaatgtat gatgccttat tgaataaatc aggggcaaca | 960 |
| ggtgtattta ctgatttccc agatactgg | 989 |

SEQ ID NO: 42
<210> 4
<211> 989
<212> DNA
<213> Haemophilus influenzae
<400> 4

| | |
|---|---|
| gcgaataccc aaatgaaatc agacaaaatc attattgctc accgtggtgc tagcggttat | 60 |
| ttaccagagc atacgttaga atctaaagca cttgcgtttg cacaacacgc agattattta | 120 |
| gagcaagatt tagcaatgac taaggatggt cgtttagtgg ttattcacga tcacttttta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgactga aaactttgaa | 300 |
| accaaagacg gcaaacaagc gcaagtttat cctaatcgtt tcccactttg gaaatcacat | 360 |
| tttagaattc acacctttga agatgaaatt gagtttatcc aaggcttaga aaaatcgact | 420 |
| ggcagaaaag tagggattta tccagaaatc aaagcacctt ggttccacca tcaaaatggc | 480 |
| aaagatattg cagctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggaatgga tttaaaatta gttcaattaa ttgcttatac agattggaaa | 660 |
| gaaacacaag aaaaagaccc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactgccc | 900 |
| gagtttttca cagacgtaaa tcaaatgtat gatgccttat tgaataaatc aggggcaaca | 960 |
| ggtgtattta ctgatttccc agatactgg | 989 |

SEQ ID NO: 43
<210> 5
<211> 989
<212> DNA
<213> Haemophilus influenzae
<400> 5

| | |
|---|---|
| gcaaataccc aaatgaaatc tgacaaaatc atcattgctc atcgtggtgc tagcggttat | 60 |
| ttaccagagc atacgttaga atctaaagca cttgcgtttg cacagcacgc tgattactta | 120 |
| gagcaagatt tagcaatgac taaggatggt cgtttagtgg ttattcacga tcacttttta | 180 |
| gatggcttga ctgatgttgc gaaaaaattc ccacatcgtc accgtaaaga tggtcgttac | 240 |
| tatgtcatcg actttacctt aaaagaaatt caaagtttag aaatgacaga aaactttgaa | 300 |
| accaaagatg gcaaacagac acaagtttat cctaatcgtt tccccctttg gcaatcccat | 360 |
| ttccgtattc acacctttga agatgaaatt gaatttattc aaggtttaga aaaatcgacg | 420 |
| ggcaaaaaag tagggattta tccagaaatc aaagcacctt ggttccacca tcaaaatggc | 480 |
| aaagatattg ctgctgaaac gctcaaagtg ttaaaaaaat atggctatga taagaaaacc | 540 |
| gatatggttt acttacaaac tttcgatttt aatgaattaa aacgtatcaa aacggaatta | 600 |
| cttccacaaa tgggtatgga tttgaaatta gttcaattaa ttgcttatac agattggaaa | 660 |
| gaaacacaag aaaaagattc aaagggttat tgggtaaact ataattacga ttggatgttt | 720 |
| aaacctggtg caatggcaga agtggttaaa tatgccgatg gtgttggccc aggttggtat | 780 |
| atgttagtta ataaagaaga atccaaacct gataatattg tgtacactcc gttggtaaaa | 840 |
| gaacttgcac aatataatgt ggaagtgcat ccttacaccg tgcgtaaaga tgcactacct | 900 |
| gcgtttttca cagacgtaaa tcaaatgtat gatgccttat tgaataaatc aggggcaaca | 960 |
| ggtgtattta ctgatttccc agatactgg | 989 |

SEQ ID NO: 44
<210> 6
<211> 1259
<212> DNA
<213> H.influenzae
<400> 6

| | |
|---|---|
| acctacggta ctaaataatt agcttaaaaa aggcggcggg caaattgctt agtcgccttt | 60 |
| tttgtaacta aaatctaaaa aaaaccataa aaatttaccg cactcttaag gagaaaatac | 120 |
| ttatgaaact taaaacttta gccctttctt tattagcagc tggcgtacta gcaggttgta | 180 |
| gcagccattc atcaaatatg gcgaataccc aaatgaaatc agacaaaatc attattgctc | 240 |
| accgtggtgc tagcggttat ttaccagagc atacgttaga atctaaagca cttgcgtttg | 300 |
| cacaacaggc tgattattta gagcaagatt tagcaatgac taaggatggt cgtttagtgg | 360 |
| ttattcacga tcacttttta gatggcttga ctgatgttgc gaaaaaattc ccacatcgtc | 420 |
| accgtaaaga tggccgttac tatgtcatcg actttacctt aaaagaaatt caaagtttag | 480 |
| aaatgacaga aaactttgaa accaaagatg gcaaacaagc gcaagtttat cctaatcgtt | 540 |
| tcccactttg gaaatcacat tttagaattc acacctttga agatgaaatt gaatttatcc | 600 |
| aaggcttaga aaaatccact ggcaaaaaag tagggattta tccagaaatc aaagcacctt | 660 |
| ggttccacca tcaaaatggt aaagatattg ctgctgaaac gctcaaagtg ttaaaaaaat | 720 |
| atggctatga taagaaaacc gatatggttt acttacaaac tttcgatttt aatgaattaa | 780 |
| aacgtatcaa aacggaatta cttccacaaa tgggtatgga tttgaaatta gttcaattaa | 840 |
| ttgcttatac agattggaaa gaaacacaag aaaaagatcc aaagggttat tgggtaaact | 900 |
| ataattacga ttggatgttt aaacctggag caatggcaga agtggttaaa tatgccgatg | 960 |
| gtgttggtcc aggttggtat atgttagtta ataaagaaga atccaaacct gataatattg | 1020 |
| tgtacactcc gttggtaaaa gaacttgcac aatataatgt ggaagtgcat ccttacaccg | 1080 |
| tgcgtaaaga tgcactaccc gcgtttttca cagatgtaaa tcaaatgtat gatgccttat | 1140 |
| tgaataaatc aggggcaaca ggtgtattta ctgatttccc agatactggc gtggaattct | 1200 |
| taaaaggaat aaaataatat ccctcacaac cgtgggtaaa catacccacg ttaactagg | 1259 |

SEQ ID NO: 45
<210> 7
<211> 1258
<212> DNA
<213> H.influenzae
<400> 7

| | |
|---|---|
| acttacggta ctaaataatt agcttaaaaa aggcggtggg taaattgctt agtcgccttt | 60 |
| tttgtaacta aaatctaaaa aaaccataaa aatttaccgc actcttaagg agaaaatact | 120 |
| tatgaaactt aaaactttag ccctttcttt attagcagct ggcgtactag caggttgtag | 180 |
| cagccattca tcaaatatgg cgaataccca aatgaaatca gacaaaatca ttattgctca | 240 |
| ccgtggtgct agcggttatt taccagagca tacgttagaa tctaaagcac ttgcgtttgc | 300 |
| acaacaggct gattatttag agcaagattt agcaatgact aaggatggtc gtttagtggt | 360 |
| tattcacgat cactttttag atggcttgac tgatgttgcg aaaaaattcc cacatcgtca | 420 |
| ccgtaaagat ggtcgttact atgtcatcga ctttacctta aaagaaattc aaagtttaga | 480 |
| aatgacagaa aactttgaaa ccaaagacgg caaacaagcg caagtttatc ctaatcgttt | 540 |
| cccactttgg aaatcacatt ttagaattca tacctttgaa gatgaaattg aatttatcca | 600 |
| aggcttagaa aaatccactg gcaaaaaagt agggatttat ccagaaatca aagcaccttg | 660 |
| gttccaccat caaaatggta aagatattgc tgctgaaacg ctcaaagtgt taaaaaaata | 720 |
| tggctatgat aagaaaaccg atatggttta cttacaaact ttcgatttta atgaattaaa | 780 |
| acgtatcaaa acggaattac ttccacaaat ggggatggat ttgaaattag ttcaattaat | 840 |
| tgcttataca gattggaaag aaacacaaga aaaagaccca aagggttatt gggtaaacta | 900 |
| taattacgat tggatgttta aacctggagc aatggcagaa gtggttaaat atgccgatgg | 960 |
| tgttggtcca ggttggtata tgttagttaa taaagaagaa tccaaacctg ataatattgt | 1020 |
| gtacactccg ttggtaaaag aacttgcaca atataatgtg gaagtgcatc cttacaccgt | 1080 |
| gcgtaaagat gcactgcccg agtttttcac agacgtaaat caaatgtatg atgtcttatt | 1140 |
| gaataaatca ggggcaacag gtgtatttac tgatttccca gatactggcg tggaattctt | 1200 |
| aaaaggaata aaataatatc cctcacaacc gtgggtaaac atacccacgt taactagg | 1258 |

SEQ ID NO: 46
<210> 8
<211> 1252
<212> DNA
<213> H.influenzae
<400> 8

| | |
|---|---|
| acttacggta ctaaataatt agcttaaaaa aggcggtggg caaattgctt agtcgccttt | 60 |
| tttgtaacta aaatctaaaa aaaccataaa aatttaccgc actttcaagg agaaaatact | 120 |
| tatgaaactt aaaactttag ccctttcttt attagcagct ggcgtactag caggttgtag | 180 |
| cagccattca tcaaatatgg cgaaaaccca aatgaaatca gacaaaatca ttattgctca | 240 |
| ccgtggtgct agcggttatt taccagagca tacgttagaa tctaaagcac ttgcgtttgc | 300 |
| acaacaggct gattatttag agcaagattt agcaatgact aaggatggtc gtttagtggt | 360 |
| tattcacgat cactttttag atggcttgac tgatgttgcg aaaaaattcc cacatcgtca | 420 |
| ccgtaaagat ggtcgttact atgtcatcga ctttacctta aaagaaattc aaagtttaga | 480 |
| aatgacagaa aactttgaaa ccaaagacgg caaacaagcg caagtttatc ctaatcgttt | 540 |
| ccccctttgg caatcccatt tccgtattca cacctttgaa gatgaaattg aatttatcca | 600 |
| aggcttagaa aaatcgactg gcagaaaagt agggatttat ccagaaatca aagcaccttg | 660 |
| gttccaccat caaaatggta aagatattgc tgctgaaacg ctcaaagtgt tgaaaaaata | 720 |
| tggctatgat aagaaaaccg atatggttta cttacaaact ttcgacttta atgaattaaa | 780 |
| acgtatcaaa acggaattac ttccacaaat gggtatggat ttgaaattag ttcaattaat | 840 |
| tgcttataca gattggaaag aaacacaaga aaaagattca aagggttatt gggtaaacta | 900 |
| taattacgat tggatgttta aacctggtgc aatggcagaa gtggttaaat atgccgatgg | 960 |
| tgttggccca ggttggtata tgttagttaa taaagaagaa tccaaacctg ataatattgt | 1020 |
| gtacactccg ttggtaaaag aacttgcaaa atataatgtg gaagtgcatc cttacaccgt | 1080 |
| gcgtaaagat gcactgcctg cgtttttcac agacgtaaat caaatgtatg atgctttatt | 1140 |
| gaataaatca ggggcaacag gtgtatttac tgatttccca gatactggcg tggaattctt | 1200 |
| aaaaggaata gaataatatc cctcacaacc gtgggtaaac atacccacgg tt | 1252 |

SEQ ID NO: 47
<210> 9
<211> 1864
<212> DNA
<213> Haemophilus influenzae
<400> 9

| | |
|---|---|
| gatcggcggt ggcgtattag cggtgttatt actcttaatc gtaatggttg aagaaggaaa | 60 |
| acacaaagcg aaattaggcg atacttacgg tactaaataa ttagcttaaa aaaggcggtg | 120 |
| ggcaaattgc ttagtcgcct tttttgtaac taaaatctaa aaactctata aaaatttacc | 180 |
| gcactcttaa ggagaaaata cttatgaaac ttaaaacttt agccctttct ttattagcag | 240 |
| ctggcgtact agcaggttgt agcagccatt catcaaatat ggcgaatacc caaatgaaat | 300 |
| cagacaaaat cattattgct caccgtggtg ctagcggtta tttaccagag catacgttag | 360 |
| aatctaaagc acttgcgttt gcacaacagg ctgattattt agagcaagat ttagcaatga | 420 |
| ctaaggatgg tcgtttagtg gttattcacg atcacttttt agatggcttg actgatgttg | 480 |
| cgaaaaaatt cccacatcgt catcgtaaag atggccgtta ctatgtcatc gactttacct | 540 |
| taaaagaaat tcaaagttta gaaatgacag aaaactttga aaccaaagat ggcaaacaag | 600 |
| cgcaagttta tcctaatcgt ttccctcttt ggaaatcaca ttttagaatt catacctttg | 660 |
| aagatgaaat tgaatttatc caaggcttag aaaaatccac tggcaaaaaa gtagggattt | 720 |
| atccagaaat caaagcacct tggttccacc atcaaaatgg taaagatatt gctgctgaaa | 780 |
| cgctcaaagt gttaaaaaaa tatggctatg ataagaaaac cgatatggtt tacttacaaa | 840 |
| ctttcgattt taatgaatta aaacgtatca aaacggaatt acttccacaa atgggaatgg | 900 |
| atttgaaatt agttcaatta attgcttata cagattggaa agaaacacaa gaaaaagacc | 960 |
| caaagggtta ttgggtaaac tataattacg attggatgtt taaacctggt gcaatggcag | 1020 |
| aagtggttaa atatgccgat ggtgttggcc caggttggta tatgttagtt aataaagaag | 1080 |
| aatccaaacc tgataatatt gtgtacactc cgttggtaaa agaacttgca caatataatg | 1140 |
| tggaagtgca tccttacacc gtgcgtaaag atgcactgcc cgagtttttc acagacgtaa | 1200 |
| atcaaatgta tgatgcctta ttgaataaat caggggcaac aggtgtattt actgatttcc | 1260 |
| cagatactgg cgtggaattc ttaaaaggaa taaaataata tccctcacaa ccgtgggtaa | 1320 |
| acatacccac ggttaactag gtttctatat cgtagaaact aaaaatctac tctaacagag | 1380 |
| taacatcata atcaatctag gtgttctaac ctagaattca aataaggagg ctatttcaaa | 1440 |
| acactccgta ttctttttta ataaattctc ttccctttac ttagggaaaa cactcttcat | 1500 |
| ttcaaccgca cttctaagga gtgctctatg gataaatcat taaaagcgaa ctgtattggc | 1560 |
| gagtttttag gtacagcctt attgattttc tttggtgtgg gctgcgttgc agcactaaaa | 1620 |
| gtagcaggcg ctagttttgg cttgtgggaa atcagcatta tgtgggggat gggcgttgca | 1680 |
| cttgcagtat atgcaacagc gggtttatct ggcgcacatt taaaccctgc agtaaccatt | 1740 |
| gccctttgga aatttgcttg ctttgatggc aaaaaagtaa ttccttacat catttcacaa | 1800 |
| atgctcggcg cattctttgc tgccgcatta gtttatgcct tataccgcaa tgtttttatc | 1860 |
| gatc | 1864 |

```
SEQ ID NO: 48
210> 10
<211> 25
<212> PRT
<213> H. influenzae
<400> 10
Pro Lys Arg Tyr Ala Arg Ser Val Arg Gln Tyr Lys Ile Leu Asn Cys
1 5 10 15
Ala Asn Tyr His Leu Thr Gln Val Arg
              20 25
SEQ ID NO: 49
<210> 11
<211> 160
<212> PRT
<213> H. influenzae
<400> 11
Met Lys Lys Ile Ile Leu Thr Leu Ser Leu Gly Leu Leu Thr Ala Cys
1 5 10 15
Ser Ala Gln Ile Gln Lys Ala Glu Gln Asn Asp Val Lys Leu Ala Pro
              20 25 30
Pro Thr Asp Val Arg Ser Gly Tyr Ile Arg Leu Val Lys Asn Val Asn
          35 40 45
Tyr Tyr Ile Asp Ser Glu Ser Ile Trp Val Asp Asn Gln Glu Pro Gln
    50 55 60
Ile Val His Phe Asp Ala Val Val Asn Leu Asp Lys Gly Leu Tyr Val
65 70 75 80
Tyr Pro Glu Pro Lys Arg Tyr Ala Arg Ser Val Arg Gln Tyr Lys Ile
                  85 90 95
Leu Asn Cys Ala Asn Tyr His Leu Thr Gln Val Arg Thr Asp Phe Tyr
              100 105 110
Asp Glu Phe Trp Gly Gln Gly Leu Arg Ala Ala Pro Lys Lys Gln Lys
        115 120 125
Lys His Thr Leu Ser Leu Thr Pro Asp Thr Thr Leu Tyr Asn Ala Ala
    130 135 140
Gln Ile Ile Cys Ala Asn Tyr Gly Glu Ala Phe Ser Val Asp Lys Lys
145 150 155 160
SEQ ID NO: 50
<210> 12
<211> 149
<212> PRT
<213> H. influenzae
<400> 12
Met Lys Leu Thr Thr Gln Gln Thr Leu Lys Lys Gly Phe Thr Leu Ile
1 5 10 15
Glu Leu Met Ile Val Ile Ala Ile Ile Ala Ile Leu Ala Thr Ile Ala
              20 25 30
Ile Pro Ser Tyr Gln Asn Tyr Thr Lys Lys Ala Ala Val Ser Glu Leu
            35 40 45
Leu Gln Ala Ser Ala Pro Tyr Lys Ala Asp Val Glu Leu Cys Val Tyr
    50 55 60
Ser Thr Asn Glu Thr Thr Asn Cys Thr Gly Gly Lys Asn Gly Ile Ala
65 70 75 80
Ala Asp Ile Thr Thr Ala Lys Gly Tyr Val Lys Ser Val Thr Thr Ser
                      85 90 95
Asn Gly Ala Ile Thr Val Lys Gly Asp Gly Thr Leu Ala Asn Met Glu
              100 105 110
Tyr Ile Leu Gln Ala Thr Gly Asn Ala Ala Thr Gly Val Thr Trp Thr
            115 120 125
Thr Thr Cys Lys Gly Thr Asp Ala Ser Leu Phe Pro Ala Asn Phe Cys
    130 135 140
Gly Ser Val Thr Gln
145
SEQ ID NO: 51
<210> 72
<211> 275
<212> PRT
<213> Artificial
<220>
<223> fusion protein LVL735 (protein)
<400> 72
Met Lys Tyr Leu Leu Pro Thr Ala Ala Ala Gly Leu Leu Leu Leu Ala
1 5 10 15
Ala Gln Pro Ala Met Ala Ile Gln Lys Ala Glu Gln Asn Asp Val Lys
              20 25 30
Leu Ala Pro Pro Thr Asp Val Arg Ser Gly Tyr Ile Arg Leu Val Lys
          35 40 45
Asn Val Asn Tyr Tyr Ile Asp Ser Glu Ser Ile Trp Val Asp Asn Gln
    50 55 60
Glu Pro Gln Ile Val His Phe Asp Ala Val Val Asn Leu Asp Lys Gly
65 70 75 80
Leu Tyr Val Tyr Pro Glu Pro Lys Arg Tyr Ala Arg Ser Val Arg Gln
                    85 90 95
Tyr Lys Ile Leu Asn Cys Ala Asn Tyr His Leu Thr Gln Val Arg Thr
                100 105 110
Asp Phe Tyr Asp Glu Phe Trp Gly Gln Gly Leu Arg Ala Ala Pro Lys
        115 120 125
Lys Gln Lys Lys His Thr Leu Ser Leu Thr Pro Asp Thr Thr Leu Tyr
    130 135 140
Asn Ala Ala Gln Ile Ile Cys Ala Asn Tyr Gly Glu Ala Phe Ser Val
145 150 155 160
Asp Lys Lys Gly Gly Thr Lys Lys Ala Ala Val Ser Glu Leu Leu Gln
                  165 170 175
Ala Ser Ala Pro Tyr Lys Ala Asp Val Glu Leu Cys Val Tyr Ser Thr
                180 185 190
Asn Glu Thr Thr Asn Cys Thr Gly Gly Lys Asn Gly Ile Ala Ala Asp
        195 200 205
Ile Thr Thr Ala Lys Gly Tyr Val Lys Ser Val Thr Thr Ser Asn Gly
      210 215 220
Ala Ile Thr Val Lys Gly Asp Gly Thr Leu Ala Asn Met Glu Tyr Ile
225 230 235 240
Leu Gln Ala Thr Gly Asn Ala Ala Thr Gly Val Thr Trp Thr Thr Thr
                  245 250 255
Cys Lys Gly Thr Asp Ala Ser Leu Phe Pro Ala Asn Phe Cys Gly Ser
              260 265 270
Val Thr Gln
          275
SEQ ID NO: 52
<210> 73
<211> 253
<212> PRT
<213> Artificial
<220>
<223> PE-PilA fusion protein without signal peptide
<400> 73
Ile Gln Lys Ala Glu Gln Asn Asp Val Lys Leu Ala Pro Pro Thr Asp
1 5 10 15
Val Arg Ser Gly Tyr Ile Arg Leu Val Lys Asn Val Asn Tyr Tyr Ile
                20 25 30
Asp Ser Glu Ser Ile Trp Val Asp Asn Gln Glu Pro Gln Ile Val His
        35 40 45
Phe Asp Ala Val Val Asn Leu Asp Lys Gly Leu Tyr Val Tyr Pro Glu
    50 55 60
Pro Lys Arg Tyr Ala Arg Ser Val Arg Gln Tyr Lys Ile Leu Asn Cys
65 70 75 80
Ala Asn Tyr His Leu Thr Gln Val Arg Thr Asp Phe Tyr Asp Glu Phe
                    85 90 95
Trp Gly Gln Gly Leu Arg Ala Ala Pro Lys Lys Gln Lys Lys His Thr
                100 105 110
Leu Ser Leu Thr Pro Asp Thr Thr Leu Tyr Asn Ala Ala Gln Ile Ile
          115 120 125
Cys Ala Asn Tyr Gly Glu Ala Phe Ser Val Asp Lys Lys Gly Gly Thr
    130 135 140
Lys Lys Ala Ala Val Ser Glu Leu Leu Gln Ala Ser Ala Pro Tyr Lys
145 150 155 160
Ala Asp Val Glu Leu Cys Val Tyr Ser Thr Asn Glu Thr Thr Asn Cys
                  165 170 175
Thr Gly Gly Lys Asn Gly Ile Ala Ala Asp Ile Thr Thr Ala Lys Gly
                180 185 190
Tyr Val Lys Ser Val Thr Thr Ser Asn Gly Ala Ile Thr Val Lys Gly
          195 200 205
Asp Gly Thr Leu Ala Asn Met Glu Tyr Ile Leu Gln Ala Thr Gly Asn
    210 215 220
Ala Ala Thr Gly Val Thr Trp Thr Thr Thr Cys Lys Gly Thr Asp Ala
225 230 235 240
Ser Leu Phe Pro Ala Asn Phe Cys Gly Ser Val Thr Gln
                  245 250
MC-009
SEQ ID NO: 53
MAKNDITLEDLPYLIKKIDQNELEADIGDITALEKYLALSQYGNILALEELNKALEELDEDVGWNQN
DIANLEDDVETLTKNQNALAEQGEAIKEDLQGLADFVEGQEGKILQNETSIKKNTQRNLVNGFEIE
KNKDAIAKNNESIEDLYDFGHEVAESIGEIHAHNEAQNETLKGLITNSIENTNNITKNKADIQALEN
NVVEELFNLSGRLIDQKADIDNNINNIYELAQQQDQHSSDIKTLKKNVEEGLLELSGHLIDQKTDIA
QNQANIQDLATYNELQDQYAQKQTEAIDALNKASSENTQNIEDLAAYNELQDAYAKQQTEAIDAL
NKASSENTQNIEDLAAYNELQDAYAKQQTEAIDALNKASSENTQNIAKNQADIANNINNIYELAQQ
QDQHSSDIKTLAKASAANTDRIAKNKADADASFETLTKNQNTLIEKDKEHDKLITANKTAIDANKA
SADTKFAATADAITKNGNAITKNAKSITDLGTKVDGFDSRVTALDTKVNAFDGRITALDSKVENG
MAAQAAHH
UspA1 with signal peptide (Genbank Accession AAD43461.1)
SEQ ID NO: 54
MNKIYKVKKNAAGHLVACSEFAKGHTKKAVLGSLLIVGILGMATTASAQKVGKATNKISGGDNNTA
NGTYLTIGGGDYNKTKGRYSTIGGGLFNEATNEYSTIGSGGYNKAKGRYSTIGGGGYNEATNQYS
TIGGGDNNTAKGRYSTIGGGGYNEATIENSTVGGGGYNQAKGRNSTVAGGYNNEATGTDSTIAG
GRKNQATGKGSFAAGIDNKANADNAVALGNKNTIEGENSVAIGSNNTVKKGQQNVFILGSNTDTT
NAQNGSVLLGHNTAGKAATIVNSAEVGGLSLTGFAGASKTGNGTVSVGKKGKERQIVHVGAGEIS
DTSTDAVNGSQLHALATVVAQNKADIKDLDDEVGLLGEEINSLEGEIFNNQDAIAKNQADIKTLESN
VEEGLLDLSGRLLDQKADIDNNINNIYELAQQQDQHSSDIKTLKNNVEEGLLDLSGRLIDQKADLTK
DIKALESNVEEGLLDLSGRLIDQKADIAKNQADIAQNQTDIQDLAAYNELQDAYAKQQTEAIDALNK
ASSANTDRIATAELGIAENKKDAQIAKAQANENKDGIAKNQADIQLHDKKITNLGILHSMVARAVGN
NTQGVATNKADIAKNQADIANNIKNIYELAQQQDQHSSDIKTLAKVSAANTDRIAKNKAEADASFET
LTKNQNTLIEQGEALVEQNKAINQELEGFAAHADVQDKQILQNQADITTNKTAIEQNINRTVANGFEI
EKNKAGIATNKQELILQNDRLNRINETNNHQDQKIDQLGYALKEQGQHFNNRISAVERQTAGGIAN
AIAIATLPSPSRAGEHHVLFGSGYHNGQAAVSLGAAGLSDTGKSTYKIGLSWSDAGGLSGGVGGS
YRWK
UspA1 without signal peptide (Genbank Accession AAD43461.1)
SEQ ID NO: 55
QKVGKATNKISGGDNNTANGTYLTIGGGDYNKTKGRYSTIGGGLFNEATNEYSTIGSGGYNKAKG
RYSTIGGGGYNEATNQYSTIGGGDNNTAKGRYSTIGGGGYNEATIENSTVGGGGYNQAKGRNST
VAGGYNNEATGTDSTIAGGRKNQATGKGSFAAGIDNKANADNAVALGNKNTIEGENSVAIGSNNT
VKKGQQNVFILGSNTDTTNAQNGSVLLGHNTAGKAATIVNSAEVGGLSLTGFAGASKTGNGTVSV
GKKGKERQIVHVGAGEISDTSTDAVNGSQLHALATVVAQNKADIKDLDDEVGLLGEEINSLEGEIFN
NQDAIAKNQADIKTLESNVEEGLLDLSGRLLDQKADIDNNINNIYELAQQQDQHSSDIKTLKNNVEE
GLLDLSGRLIDQKADLTKDIKALESNVEEGLLDLSGRLIDQKADIAKNQADIAQNQTDIQDLAAYNEL
QDAYAKQQTEAIDALNKASSANTDRIATAELGIAENKKDAQIAKAQANENKDGIAKNQADIQLHDKK
ITNLGILHSMVARAVGNNTQGVATNKADIAKNQADIANNIKNIYELAQQQDQHSSDIKTLAKVSAAN
TDRIAKNKAEADASFETLTKNQNTLIEQGEALVEQNKAINQELEGFAAHADVQDKQILQNQADITTN
KTAIEQNINRTVANGFEIEKNKAGIATNKQELILQNDRLNRINETNNHQDQKIDQLGYALKEQGQHF
NNRISAVERQTAGGIANAIAIATLPSPSRAGEHHVLFGSGYHNGQAAVSLGAAGLSDTGKSTYKIGL
SWSDAGGLSGGVGGSYRWK
UspA2H with signal peptide from Strain RH4 (GenBank: AGH27471.1)
SEQ ID NO: 56
MNKIYKVKKNAAGHLVACSEFAKGHTKKAVLGSLLIVGILGMATTASAQDRAGNQRNGAHSAIGG
GSINIADGDYSTIGGGHINTTKGNYSTIGGGNENKTTGNYSTIGGGYGNEAEGKHSTIGGGDENKA
KGDYSTIGGGDDNEATGTHSTIGGGDDNKATGDYSIIGGGYGNRATAEDATVSGGGYNQAAGKS
STVSGGYDNLAEGESSAIGGGELNSTIGSHSTVAGGFLNGAVGDNATVSGGEQNQAIGKYSTVSG
GYGNQATGIGSFAAGIENQANTENAVAVGKKNIINGDNSAAIGSNNTVEKGQKDVFILGSNTKDAQ
SNSVLLGNETTGKAATVVDSANVGGLGLTGFAGVSKVGNGTVSVGSQGKERQIVNVGAGEISATS
TDAVNGSQLHALATAVDNNQYDIINNQGDIERNQDDIKDLQKEVKGLDNEVGELSRDINSLHVTD
NQQDDIKELKRGVKELDKEMNVLSRDIVSLNDDVADNQADIAKNQADIKTLESNVEEGLLDLSGRL
LDQKADIANNVNHIYELAQQQDQHSSDIKTLKKNVEEGLLELSGHLIDQKSDIAQNQTDIQDLAAYN
ELQDQYAQKQTEAIDALNKASSENTQNIEDLAAYNELQDAYAKQQTEAIDALNKASSENTQNIAKN
QADIANNINNIYELAQQQDQHSSDIKTLAKASAANTDRIAKNKADADASFETLTKNQNTLIEKDKEH
DKLITANKTAIDANKASADTKFAATADAITKNGNAITKNAKSITDLGTKVDGFDGRVTALDTKVNAFD
GRITALDSKVENGMAAQAALSGLFQPYSVGKFNATAALGGYGSKSAVAIGAGYRVNPNLAFKAGA
AINTSGNKKGSYNIGVNYEF
UspA2H without signal peptide from Strain RH4 (GenBank: AGH27471.1)
SEQ ID NO: 57
QDRAGNQRNGAHSAIGGGSINIADGDYSTIGGGHINTTKGNYSTIGGGNENKTTGNYSTIGGGYG
NEAEGKHSTIGGGDENKAKGDYSTIGGGDDNEATGTHSTIGGGDDNKATGDYSIIGGGYGNRATA
EDATVSGGGYNQAAGKSSTVSGGYDNLAEGESSAIGGGELNSTIGSHSTVAGGFLNGAVGDNAT
VSGGEQNQAIGKYSTVSGGYGNQATGIGSFAAGIENQANTENAVAVGKKNIINGDNSAAIGSNNTV
EKGQKDVFILGSNTKDAQSNSVLLGNETTGKAATVVDSANVGGLGLTGFAGVSKVGNGTVSVGS
QGKERQIVNVGAGEISATSTDAVNGSQLHALATAVDNNQYDIINNQGDIERNQDDIKDLQKEVKGL
DNEVGELSRDINSLHDVTDNQQDDIKELKRGVKELDKEMNVLSRDIVSLNDDVADNQADIAKNQA
DIKTLESNVEEGLLDLSGRLLDQKADIANNVNHIYELAQQQDQHSSDIKTLKKNVEEGLLELSGHLID
QKSDIAQNQTDIQDLAAYNELQDQYAQKQTEAIDALNKASSENTQNIEDLAAYNELQDAYAKQQTE
AIDALNKASSENTQNIAKNQADIANNINNIYELAQQQDQHSSDIKTLAKASAANTDRIAKNKADADAS
FETLTKNQNTLIEKDKEHDKLITANKTAIDANKASADTKFAATADAITKNGNAITKNAKSITDLGTKVD
GFDGRVTALDTKVNAFDGRITALDSKVENGMAAQAALSGLFQPYSVGKFNATAALGGYGSKSAVA
IGAGYRVNPNLAFKAGAAINTSGNKKGSYNIGVNYEF
UspA2V with signal peptide from Strain S43:4 (GenBank: AEQ18817.1)
SEQ ID NO: 58
MKTMKLLPLKIAVTSAMIVGLGAASNANAQTSNSGTNQIVEEFLPKLTQALNDPQYQHEFEDALDQ
YHNMVVGNTSFVSTVDVANQTVGNTALKFYSDSENTLPSSMLFDQILTNQRLNGFKEGQPFIPVD
DNGNPITQVDDVLESNGQTKKVRSVTTRIATAEDVTKSPYAQGIQKDIDDIYEFNHEVVDNIATLNR
ATEILTEAGNHHDELIRKNKADIQVSNGKIESLKTLYETTVRTVGNNESNIKTNKDAIAKNKANIKTLA
QAQLESVTFLGEQIQENKDDISAVTGKAFKNQIDIQRNNDSIEDLYEANNDNVERLVEHDRAIEILAE
AGNHHDTLIRKNKADIQDLAAHNELQTEAIDALNKASSANTKRITTAELGIAENKKDALIAKAQADKN
KNDIQDLAKVQRAGVEVMAELNKNIAGTQTDIANNKITLAQHAKKIDENQQAIEAKLGGKADLQKLS
ELKTSVDKNQAGIQLHDQKINNLGILHTMVARAVGANKTAIDQNKADIAENKEDIQNNINNIYELAQQ
QDQHSSDIKTLAKASSANTDNIAKNKADADASFKTLTKNQNTLIEKDKAQDALITANKADADASFKT
LTKNQNTLIEKDKAQDKLITANKADADAKFTGVQTDIANNKTTLAQHAKKINDNQQAIEAKLGGKAD
AQALNNLNDKVNGFDGRISALDTKVNAFDGRITALDSKVENGMAAQAALSGLFQPYSVGKFNATA
ALGGYGSKSAVAIGAGYRVNPNLAIKAGAAINTSGDKKGSYNIGVNYEF
UspA2V without signal peptide from Strain S43:4 (GenBank: AEQ18817.1)
SEQ ID NO: 59
QTSNSGTNQIVEEFLPKLTQALNDPQYQHEFEDALDQYHNMVVGNTSFVSTVDVANQTVGNTALK
FYSDSENTLPSSMLFDQILTNQRLNGFKEGQPFIPVDDNGNPITQVDDVLESNGQTKKVRSVTTRI
ATAEDVTKSPYAQGIQKDIDDIYEFNHEVVDNIATLNRATEILTEAGNHHDELIRKNKADIQVSNGKI
ESLKTLYETTVRTVGNNESNIKTNKDAIAKNKANIKTLAQAQLESVTFLGEQIQENKDDISAVTGKAF
KNQIDIQRNNDSIEDLYEANNDNVERLVEHDRAIEILAEAGNHHDTLIRKNKADIQDLAAHNELQTEA
IDALNKASSANTKRITTAELGIAENKKDALIAKAQADKNKNDIQDLAKVQRAGVEVMAELNKNIAGT
QTDIANNKITLAQHAKKIDENQQAIEAKLGGKADLQKLSELKTSVDKNQAGIQLHDQKINNLGILHTM
VARAVGANKTAIDQNKADIAENKEDIQNNINNIYELAQQQDQHSSDIKTLAKASSANTDNIAKNKAD
ADASFKTLTKNQNTLIEKDKAQDALITANKADADASFKTLTKNQNTLIEKDKAQDKLITANKADADAK
FTGVQTDIANNKTTLAQHAKKINDNQQAIEAKLGGKADAQALNNLNDKVNGFDGRISALDTKVNAF
DGRITALDSKVENGMAAQAALSGLFQPYSVGKFNATAALGGYGSKSAVAIGAGYRVNPNLAIKAG
AAINTSGDKKGSYNIGVNYEF
SEQ ID NO: 60
YNELQDAYAQKQTE
SEQ ID NO: 61
YNELQDAYAKQQTE
SEQ ID NO: 62
YNELQDQYAQKQTE
SEQ ID NO: 63
YNELQDQYAKQQTE
SEQ ID NO: 64
YNELQD-[A/Q]-YA-[QK/KQ]-QTE
Amino acids 20-160 of Protein E
SEQ ID NO: 65
IQKAEQNDVKL APPTDVRSGY IRLVKNVNYY IDSESIWVDN QEPQIVHFDA VVNLDKGLYV
YPEPKRYARS VRQYKILNCA NYHLTQVRTD FYDEFWGQGL RAAPKKQKKH TLSLTPDTTL
YNAAQIICAN YGEAFSVDKK
Amino acids 40-149 of PilA from H. influenzae strain 86-028NP
SEQ ID NO: 66
TKKAAVSELLQ ASAPYKADVE LCVYSTNETT NCTGGKNGIA ADITTAKGYV KSVTTSNGAI
TVKGDGTLAN MEYILQATGN AATGVTWTTT CKGTDASLFP ANFCGSVTQ.
SEQ ID NO: 67
SSHSSNMANT
O46E uspA2H
SEQ ID NO: 68
MNKIYKVKKNAAGHSVACSEFAKGHTKKAVLGSLLIVGALGMATTASAQTGSTNAANGNIISGVGA
YVGGGVINQAKGNYPTVGGGFDNRATGNYSVISGGFDNQAKGEHSTIAGGESNQATGRNSTVAG
GSNNQAVGTNSTVAGGSNNQAKGANSFAAGVGNQANTDNAVALGKNNTINGNNSAAIGSENTVN
ENQKNVFILGSNTTNAQSGSVLLGHETSGKEATAVSRARVNGLTLKNFSGVSKADNGTVSVGSQ
GKERQIVHVGAGQISDDSTDAVNGSQLYALATAVDDNQYDIEINQDNIKDLQKEVKGLDKEVGVLS
RDIGSLHDDVADNQADIAKNKADIKELDKEMNVLSRDIVSLNDDVADNQADIAKNQADIKTLENNVE
EGLLDLSGRLIDQKADIDNNINHIYELAQQQDQHSSDIKTLKKNVEEGLLELSGHLIDQKADIAQNQT
DIQDLATYNELQDQYAQKQTEAIDALNKASSENTQNIEDLAAYNELQDAYAKQQTEAIDALNKASS
ENTQNIEDLAAYNELQDAYAKQQTEAIDALNKASSENTQNIEDLAAYNELQDAYAKQQTEAIDALN
KASSENTQNIQDLAAYNELQDAYAKQQTEAIDALNKASSENTQNIQDLAAYNELQDAYAKQQTEAI
DALNKASSENTQNIAKNQADIANNINNIYELAQQQDQHSSDIKTLAKASAANTDRIAKNKADADASF
ETLTKNQNTLIEKDKEHDKLITANKTAIDANKASADTKFAATADAITKNGNAITKNAKSITDLGTKVDG
FDGRVTALDTKVNAFDGRITALDSKVENGMAAQAALSGLFQPYSVGKFNATAALGGYGSKSAVAI
GAGYRVNPNLAFKAGAAINTSGNKKGSYNIGVNYEF
E22USPA2H
SEQ ID NO: 69
MNKIYKVKKNAAGHSVACSEFAKGHTKKAVLGSLLIVGALGMATTASAQLVSTTQPNDYRSSTTG
NNHLGSSWSIIGAGHDNIVYRSASNSGILSGYKNRVNGSTSAIVGGYDNETRGKYTFVGGGYKNL
AEGHQSAIGGGYANWAEGDNATIAGGFENFAAGNQSAIGGGYANLAEGDDATIAGGFENRAEGN
QSAIGGGYANFAAGDYTFVGGGYENRAEGNQSAIGGGYANLAEGDNATIAGGFENRAKGINSVV
SGGYANQATGESSTIAGGFENRAEGIDSVVSGGYANQANGAQSTVAGGYNNQATGESSTIAGGS
NNQATGTGSFAAGVENKANADNAVALGKNNIINGDNSAAIGSNNTVKKGQKDVFILGSNTSGAQS
NSVLLGNETTGKKATAVENATVGDLSLTGFAGVSKANSGTVSVGSEGKERQIVHVGAGRISNDST
DAVNGSQLYALAAAVDDNQYDIEKNQDDIKELKRGVKELDKEMNVLSRDIVSLNDDVAQNQSDIKT
LKNNVEEGLLELSGHLIDQKADLTKDIKALENNVEEGLLDLSGRLLDQKADIAKNQADIAQNQTDIQ
DLAAYNELQDAYAKQQTEAIDALNKASSENTQNIEDLAAYNELQDAYAKQQTEAIDALNKASSENT
QNIAKNQADIANNINNIYELAQQQDQHSSDIKTLAKASAANTDRIAKNKADADASFETLTKNQNTLIE
KDKEHDKLITANKTAIDANKASADTKFAATADAITKNGNAITKNAKSITDLGTKVDGFDGRVTALDTK
VNAFDGRITALDSKVENGMAAQAALSGLFQPYSVGKFNATAALGGYGSKSAVAIGAGYRVNPNLA
FKAGAAINTSGNKKGSYNIGVNYEF
Mcat454epiUspA2H
SEQ ID NO: 70
VNKIYKVKKNVAGHSVACSEFAKGHTKKAVLGSLLIVGALGMATTASAQQNNKGFGAYIGVENSG
NLATGQYSVAVGGLGNEATGQYSVINGGYFNIADGVHSIIGGGKDNKAKGNYSTIGGGDSNKAEG
NYSTIAGGRNNEATGEGSFAAGIDNKANAKNAVALGNKNSIEGTNSVAIGSNNTVAQDNTFILGSN
TTGVQRNSVLLGNNTAGQEATIVEKAEVGGVNLTGFAGVSKTSNGTVSVGSKNNERQIVNVGAG
RISKDSTDAVNGSQLYALATAVDDSQYDIEKNQDDIEKNQDDIKELKRGVKDLKKGVKGLDKEVKD
LNKKVGVLDRDIGSLQDDVADNQADIANNQADIKDLDKEVGVLSRDIGSLHDDVAQNQSDITLKN
NVEEGLLELSGHLLDQKADIDNNINNIYELAQQQDQHSSDIKTLKKNVEEGLLELSGHLIDQKADIAQ
NQTDIQDLATYNELQDQYAQKQTEAIDALNKASSENTQNIAKNSNRIKALESNVEEGLLELSGHLID
QKADLTKDIKALESNVEEGLLDLSGRLIDQKADIAQNQANIQDLAAYNELQDQYAQKQTEAIDALNK
ASSENTQNIEDLAAYNELQDAYAKQQTEAIDALNKASSENTQNIAKNQADIANNINNIYELAQQQDQ
HSSDIKTLAKASAANTDRIAKNKADADASFETLTKNQNTLIEKDKEHDKLITANKTAIDANKASADTK
FAATADAITKNGNAITKNAKSITDLGTKVDGFDSRVTALDTKVNAFDGRITALDSKVENGMAAQAAL
SGLFQPYSVGKFNATAALGGYGSKSAVAIGAGYRVNPNLAFKAGAAINTSGNKKGSYNIGVNYEF
Mcat415epiUspA2H
SEQ ID NO: 71
VNKIYKVKKNVAGHSVACSEFAKGHTKKAVLGSLLIAGALGMATTASAQVVGNTTEGLGARVNGG
DGNEAKGTYPTIGGGFFNIATGNYSTISGGNTNEAEGNYSTIGGGDTNAASGNNSTVVGGYNNTA
KGNYSTVAGGANNQANSIDSTIVGGRKNRAKGKSSTVSGGYDNLAGGKSSAIGGGEFNSAIGSRS
TVAGGELNLASGDNATVSGGKQNQAIGEYSTVAGGELNLASGDNATVSGGKQNQAEGTDSTIAG
GRNNQAIGEGSFAAGIDNKANTENAVAVGKKNIINGDNSAAIGSNNTVKKGQTGVFILGSNTKGAQ
SNSVLLGNETTGKKATAVSSATVNGLTLENFAGVSQVGNGTVSVGSKGKERQIVNVGAGKISATS
TDAVNGSQLHALATAVAYNYTDIIDNQADIAKNQDDIDDLNKKVGVLDRDIGSLQDDVADNQADIAN
NQADIKDLDDEVGVLSRDIGSLHDDVAQNQSDIKTLKNNVEEGLLDLSGRLLDQKADIDNNINNIYE
LAQQQDQHSSDIKTLKKNVEEGLLELSGHLIDQKSDIAKNQADIAQNQTDIHDLAAYNELQDAYAK
QQTEAIDALNKASSENTQNIEDLAAYNELQDAYAKQQTEAIDALNKASSENTQNIAKNQADIANNIN
NIYELAQQQDQHSSDIKTLAKASAANTDRIAKNKADADASFETLTKNQNTLIEKDKEHDKLITANKTA
IDANKASADTKFAATADAITKNGNAITKNAKSITDLGTKVDAFDGRVTALDTKVNALDTKVNAFDGRI
TALDSKVENGMAAQAALSGLFQPYSVGKFNATAALGGYGSKSAVAIGAGYRVNPNLAFKAGAAIN
TSGNKKGSYNIGVNYEF
RH4UspA2H
SEQ ID NO: 72
VNKIYKVKKNAAGHLVACSEFAKGHTKKAVLGSLLIVGILGMATTASAQDRAGNQRNGAHSAIGGG
SINIADGDYSTIGGGHINTTKGNYSTIGGGNENKTTGNYSTIGGGYGNEAEGKHSTIGGGDENKAK
GDYSTIGGGDDNEATGTHSTIGGGDDNKATGDYSIIGGGYGNRATAEDATVSGGGYNQAAGKSS
TVSGGYDNLAEGESSAIGGGELNSTIGSHSTVAGGFLNGAVGDNATVSGGEQNQAIGKYSTVSG
GYGNQATGIGSFAAGIENQANTENAVAVGKKNIINGDNSAAIGSNNTVEKGQKDVFILGSNTKDAQ
SNSVLLGNETTGKAATVVDSANVGGLGLTGFAGVSKVGNGTVSVGSQGKERQIVNVGAGEISATS
TDAVNGSQLHALATAVDNNQYDIINNQGDIERNQDDIKDLQKEVKGLDNEVGELSRDINSLHDVTD
NQQDDIKELKRGVKELDKEMNVLSRDIVSLNDDVADNQADIAKNQADIKTLESNVEEGLLDLSGRL
LDQKADIANNVNHIYELAQQQDQHSSDIKTLKKNVEEGLLELSGHLIDQKSDIAQNQTDIQDLAAYN
ELQDQYAQKQTEAIDALNKASSENTQNIEDLAAYNELQDAYAKQQTEAIDALNKASSENTQNIAKN
QADIANNINNIYELAQQQDQHSSDIKTLAKASAANTDRIAKNKADADASFETLTKNQNTLIEKDKEH
DKLITANKTAIDANKASADTKFAATADAITKNGNAITKNAKSITDLGTKVDGFDGRVTALDTKVNAFD
GRITALDSKVENGMAAQAALSGLFQPYSVGKFNATAALGGYGSKSAVAIGAGYRVNPNLAFKAGA
AINTSGNKKGSYNIGVNYEF
Mcat031epiUspA2
SEQ ID NO: 73
MKTMKLLPLKIAVTSALIVGLGAASTANAQKKASSQELNQHIRVLNKYIRDFNDDIQELDYDIQDLND
DIKGLDKKLSRDINSLRDEVTDNYTDIIDNQVDIERNQADIKDLKRGVKGLNKEVGVLDRDIGSLQD
DVADNQADIANNQDDIKDLDDEVGVLSRDIGSLHDDVAQNQSDIKTLESNVEEGLLELSGHLIDQK
ADIDNNINHIYELAQQQDQHSSDIKTLKKNVEEGLLELSGHLIDQKSDIAQNQTDIQDLATYNELQD
QYAQKQTEAIDALNKASSENTQNIQDLAAYNELQDAYAKQQTEAIDALNKASSENTQNIEDLAAYN
ELQDQYAQKQTEAIDALNKASSENTQNIQDLAAYNELQDAYAKQQTEAIDALNKASSENTQNIEDL
AAYNELQDQYAQKQTEAIDALNKASSENTQNIAKNQADIANNINNIYELAQQQDQHSSDIKTLAKAS
AANTDRIAKNKADADASFETLTKNQNTLIEKDKEHDKLITANKTAIDANKASADTKFAATADAITKNG
NAITKNAKSITDLGTKVDGFDGRVTALDTKVNAFDGRITALDSKVENGMAAQAALSGLFQPYSVGK
FNATAALGGYGSKSAVAIGAGYRVNPNLAFKAGAAINTSGNKKGSYNIGVNYEF
MC2908uspA2
SEQ ID NO: 74
MKTMKLLPLKIAVTSALIVGLGAASTANAQLVERFFPNIFLDKPLAKQHYHNVVVGDTSIVSDLQSN
SDQLKFYSDDEGLVPDSLLFNKMLHEQLLNGFKEGDTIIPLDENGKPVYKVDYKLDGKEPRKVYSV
TTKIATAEDVATSSYANGIQKDIDDLYDFDHQVTERLTQHGKTIYRNGERILANEESVQYLNKEVQN
NIEHIYELAQQQDQHSSDIKTLESNVEKGLLELSGHLIDQKADLTKDIKTLESNVEEGLLDLSGRLID
QKADLTKDIKTLESNVEEGLLDLSGRLIDQKADIAQNQANIQDLAAYNELQDQYAQKQTEAIDALNK
ASSENTQNIEDLAAYNELQDAYAKQQTEAIDALNKASSENTQNIAKNQADIANNINNIYELAQQQDQ
HSSDIKTLAKASAANTNRIATAELGIAENKKDAQIAKAQANANKTAIDENKASADTKFAATADAITKN
GNAITKNAKSITDLGTKVDGFDSRVTALDTKVNAFDGRITALDSKVENGMAAQAALSGLFQPYSVG
KFNATAALGGYGSKSAVAIGAGYRVNPNLAFKAGAAINTSGNKKGSYNIGVNYEF
Mox307uspa2
SEQ ID NO: 75
MKTMKLLPLKIAVTSAMIIGLGAASTANAQQQQQQQQQQQSRTEIFFPNIFFNENHDELDDAYHNII
LGDTALLDKQDGSQPQLKFYSNDKDSVPDSLLFSKLLHEQQLNGFKKGDTIIPLDKDGKPVYQVDY
KLDGKGKKQKRRQVYSVTTKTATDDDVNSAYSRGILGKVDDLDDEMNFLNHDITSLYDVTANQQD
AIKGLKKGVKGLNKELKELDKEVGVLSRDIGSLNDDVAQNNESIEDLYDFSQEVADSIGEIHAHNKA
QNETLQDLITNSVENTNNITKNKADIQALENNVVEELFNLSGRLIDQKADLTKDIKTLESNVEEGLLE
LSGHLIDQKADIAKNQADIAQNQANIQDLAAYNELQDAYAKQQTEAIDALNKASSENTQNIEDLAAY
NELQDAYAKQQTEAIDALNKASSENTQNIEDLAAYNELQDAYAKQQTEAIDALNKASSENTQNIAK
NQADIANNINNIYELAQQQDQHSSDIKTLAKASAANTDRIAKNKADADASFETLTKNQNTLIEKDKE
HDKLITANKTAIDENKASADTKFAATADAITKNGNAITKNAKSITDLGTKVDAFDGRVTALDTKVNAF
DGRITALDSKVENGMAAQAALSGLFQPYSVGKFNATAALGGYGSKSAVAIGAGYRVNPNLAFKAG
AAINTSGNKKGSYNIGVNYEF
SEQ ID NO: 76
NxxQDxx, SxxNTxx, QxxHxxx,
and
QxxDxxx
SEQ ID NO: 77
SxxNTxx
SEQ ID NO: 78
QxxHxxx
SEQ ID NO: 79
QxxDxxx
SEQ ID NO: 80
DLAAYNELQD[A/Q]YA[KQ/QK]QTEAIDA
(Variable Heavy [VH] Chain - FHUSPA2/10)
SEQ ID NO: 81
GAGGTTCAGCTCCAGCAGTCTGGGACTGTGCTGGCAAGGCCTGGGGCTTCCGTGAAGATGT
CCTGCAAGGCTTCTGGCTACAGCTTTACCAGCTACTGGATGCACTGGGTAAAACAGAGGCCT
GGACAGGGTCTAGAATGGATTGGTGCTATTTATCCTGGAAATAGTGATACTAGCTACAACCAG
AAGTTCAAGGGCAAGGCCAAACTGACTGCAGTCACATCCGCCAGCACTGCCTACATGGAGCT
CAGCAACCTGACGAATGAGGACTCTGCGGTCTATTCCTGTACATTACTACGTTTCCTCGATGC
TTACTGGGGCCAAGGGACTCTGGTCACTGTCTCTGCAG
(VH Chain of FHUSPA2/10 - AA Seq)
SEQ ID NO: 82
EVQLQQSGTVLARPGASVKMSCKASGYSFTSYWMHWVKQRPGQGLEWIGAIYPGNSDTSYNQK
FKGKAKLTAVTSASTAYMELSNLTNEDSAVYSCTLLRFLDAYWGQGTLVTVSA
(Variable Light [VL] Chain - FHUSPA2/10)
SEQ ID NO: 83
CAAATTGTTCTCTCCCAGTCTCCAGCAATCCTGTCTGCATCTCCAGGGGAGAAGGTCACAATG
ACTTGCAGGGCCAGCTCAAGTGTAAGTTACATGCACTGGTACCAGCAGAAGCCAGGATCCTC
CCCCAAACCCTGGATTTATGCCACATCCAACCTGGCTTCTGGAGTCCCTGCTCGCTTCAGTGG
CAGTGGGTCTGGGACCTCTTACTCTCTCACAATCAGCAGAGTGGAGGCTGAAGATGCTGCCA
CTTATTACTGCCAGCAGTGGAGTAGTAACCCACCCACGTTCGGAGGGGGGTCCAAGCTGGAA
ATAAAA
(VL Chain of FHUSPA2/10 - AA Seq)
SEQ ID NO: 84
QIVLSQSPAILSASPGEKVTMTCRASSSVSYMHWYQQKPGSSPKPWIYATSNLASGVPARFSGSG
SGTSYSLTISRVEAEDAATYYCQQWSSNPPTFGGGSKLEIK
(CDRH1)
SEQ ID NO: 85
SYWMH
(CDRH2)
SEQ ID NO: 86
AIYPGNSDTSYNQKFKG
(CDRH3)
SEQ ID NO: 87
LRFLDAY
(CDRL1)
SEQ ID NO: 88
RASSSVSYMH
(CDRL2)
SEQ ID NO: 89
ATSNLAS
(CDRL3)
SEQ ID NO: 90
QQWSSNPPT
```

1. An immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 449 continuous amino acids (e.g. less than 445 amino acids, less than 300 amino acids, less than 150 amino acids).

2. The immunogenic fragment of UspA2 according to claim 1 wherein UspA2 has at least 80% identity (e.g. at least 85%, at least 90%, at least 95%) to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

3. The immunogenic fragment of UspA2 according to claim 1 or claim 2 wherein said fragment is less than 150 amino acids.

4. The immunogenic fragment of UspA2 according to any of claims 1-3 wherein said fragment comprises at least 14 amino acids and comprises an epitope within a consensus sequence of YNELQD-[A/Q]-YA-[QK/KQ]-QTE (SEQ ID NO: 64), e.g. SEQ ID NO: 60, 61, 62 or 63.

5. The immunogenic fragment of UspA2 according to any of claims 1-4 which is capable of generating an immune response against heterologous or non-heterologous strains of M. catarrhalis.

6. The immunogenic fragment of UspA2 according to claim 5 which is capable of generating an immune response against heterologous strain(s) of M. catarrhalis.

7. The immunogenic fragment of UspA2 according to any of claims 1-6 wherein the epitope confers cross-bactericidal activity against heterologous strain(s) of M. catarrhalis.

8. The immunogenic fragment of UspA2 according to any of claims 1-7 which is cross-protective against heterologous strain(s) of M. catarrhalis.

9. The immunogenic fragment of UspA2 according to any of claims 1-8 wherein UspA2 has at least 83%, 86%, 90%, 92%, 95%, 97%, 98%, 99% or 100% identity over the entire length to SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

10. The immunogenic fragment of UspA2 according to claims 1-9 wherein UspA2 has at least 83%, 86%, 90%, 92%, 95%, 97%, 98%, 99% or 100% identity to SEQ ID NO:1.

11. The immunogenic fragment of UspA2 according to any of claims 1-10 wherein the consensus sequence is located within the stalk region of UspA2.

12. The immunogenic fragment of UspA2 according to claim 11 wherein the consensus sequence is present at up to 15 locations within the stalk region of UspA2.

13. An antigen binding protein which binds to an epitope within a consensus sequence of YNELQD-[A/Q]-YA-[QK/KQ]-QTE (SEQ ID NO: 64), e.g. SEQ ID NO: 60, 61, 62 or 63.

14. The antigen binding protein according to claim 13 which binds to an epitope with the consensus sequence of SEQ ID NO: 60, 61, 62 or 63.

15. The antigen binding protein according to claim 13 or claim 14 which is an antibody.

16. The antibody according to claim 15 which binds to UspA2 at up to 15 locations within the stalk region of UspA2.

17. The antibody according to any of claims 15-16 which is an IgG2A mouse mAB

18. The antibody according to claim 17 which is FHUSPA2/10

19. The antibody according to any of claims 15-18 comprising: a VH region comprising a sequence at least 80% identical to the sequence of SEQ ID NO: 82; and/or a VL region comprising a sequence at least 80% identical to the sequence of SEQ ID NO: 84.

20. The antibody according to any of claims 15-19 comprising: a VH region comprising SEQ ID NO: 82; and/or a VL region comprising SEQ ID NO: 84.

21. An antigen binding protein comprising any one or a combination of CDRs selected from CDRH1, CDRH2, CDRH3 from SEQ ID NO: 82, and/or CDRL1, CDRL2, CDRL3 from SEQ ID NO: 84; or (ii) a CDR variant of (i), wherein the variant has 1, 2, or 3 amino acid modifications in each CDR, which is able to bind to an epitope within the consensus sequence of SEQ ID NO: 64 and promote bactericidal activity.

22. An antigen binding protein that binds to UspA2, and competes for binding to the consensus sequence SEQ ID NO: 64 with a reference the antibody with a VH region comprising SEQ ID NO: 82 and a VL region comprising SEQ ID NO: 84.

23. An immunogenic fragment of UspA2 according to any of claims 1-12 for use in preventing or treating an infection, disease or condition caused by M. catarrhalis.

24. An immunogenic fragment of UspA2 according to claim 23 for use in preventing or treating an infection, disease or condition caused by heterologous strain(s) of M. catarrhalis.

25. An immunogenic fragment of UspA2 for use in preventing or treating an infection disease or condition caused by heterologous strain(s) of M. catarrhalis according to claim 23 or claim 24 wherein said strain(s) comprise a surface protein having a sequence with 40%-99% identity (e.g. 40-50% identity, 45-65% identity, 50%-70% identity, 50-99% identity) to SEQ ID NO:1.

26. An immunogenic fragment of UspA2 for use in preventing or treating an infection, disease or condition caused by heterologous strain(s) of M. catarrhalis according to any of claim 23-25 wherein said strain(s) comprise UspA2H, UspA2V or a variant thereof.

27. An immunogenic fragment of UspA2 for use in preventing or treating an infection, disease or condition caused by heterologous strain(s) of M. catarrhalis according to any of claims 23-26 wherein said strain(s) are UspA1⁺/UspA2⁻.

28. An immunogenic fragment of UspA2 for use in preventing or treating an infection, disease or condition caused by heterologous strain(s) of M. catarrhalis according to any of claims 23-27 which is otitis media, pneumonia and/or acute exacerbations of chronic obstructive pulmonary disease (AECOPD).

29. An immunogenic fragment of UspA2 comprising an epitope within a consensus sequence of YNELQD-[A/Q]-YA-[QK/KQ]-QTE (SEQ ID NO: 64), e.g. SEQ ID NO: 60, 61, 62 or 63, wherein said fragment is less than 509 continuous amino acids (e.g. less than 450 amino acids, less than 300 amino acids, less than 150 amino acids) for use in eliciting cross-bactericidal activity against heterologous strain(s) of M. catarrhalis.

30. An immunogenic fragment of UspA2 for use in eliciting cross-bactericidal activity against heterologous strain(s) of M. catarrhalis according to claim 29 wherein UspA2 has at least 80% identity (e.g. at least 85%, at least 90%, at least 95%) to the entire length of SEQ ID NO: 1, SEQ ID NO: 36, SEQ ID NO: 38, SEQ ID NO: 68, SEQ ID NO: 69, SEQ ID NO: 70, SEQ ID NO: 71, SEQ ID NO: 72, SEQ ID NO: 73, SEQ ID NO: 74 or SEQ ID NO: 75.

31. An immunogenic fragment of UspA2 according to claims 1-12 for use in eliciting cross-bactericidal activity against heterologous strain(s) of M. catarrhalis according to claim 29.

32. An immunogenic fragment of UspA2 for use in eliciting cross-bactericidal activity against heterologous strain(s) of M. catarrhalis according to any of claims 29-31 wherein said strain(s) comprise UspA2H, UspA2V or a variant thereof.

33. An immunogenic fragment of UspA2 for use in eliciting cross-bactericidal activity against heterologous strain(s) of M. catarrhalis according to any of claims 29-32 wherein said strain(s) cause otitis media, pneumonia and/or acute exacerbations of chronic obstructive pulmonary disease (AECOPD).

34. An immunogenic composition comprising the immunogenic fragment of any of claims 1-12.

35. The immunogenic composition according to claim 34 wherein the immunogenic fragment is present as a fusion protein.

36. The immunogenic composition according to claim 34 or claim 35 comprising at least one antigen from Haemophilis influenzae.

37. The immunogenic composition according to claim 36 comprising Protein D or an immunogenic fragment thereof.

38. The immunogenic composition according to claim 36 or claim 37 comprising Protein E or an immunogenic fragment thereof.

39. The immunogenic composition according to any of claims 36-38 comprising PiA or an immunogenic fragment thereof.

40. The immunogenic composition according to any of claims 36-39 comprising a PE-PiA fusion protein or an immunogenic fragment of Protein E and an immunogenic fragment of PiIA.

41. A vaccine comprising the immunogenic fragment of claims 1-12 or the immunogenic composition of claims 34-40.

42. The vaccine according to claim 41 further comprising an adjuvant e.g. AS01E.

43. The vaccine according to any of claims 41-42 for use in treating or preventing an infection, disease or condition caused by M. catarrhalis, in a mammal, particularly a human.

44. The vaccine composition of claim 43 for use in the treatment or prevention of acute otitis media, pneumonia and/or acute exacerbations of chronic obstructive pulmonary disease (AECOPD).

45. A method of treatment or prevention of an infection, disease or condition caused by M. catarrhalis, in a subject in need thereof comprising administering to said subject a therapeutically effective amount of an immunogenic composition according to any of claims 34-40 or the vaccine according to any of claims 41-44.

46. A method of treatment or prevention of acute exacerbations of chronic obstructive pulmonary disease (AECOPD), pneumonia and/or otitis media in a subject in need thereof comprising administering to said subject a therapeutically effective amount of an immunogenic composition according to any of claims 34-40 or the vaccine according to any of claims 41-44.

47. The immunogenic fragment, immunogenic composition or vaccine according to any of claims 1-46 for use in the manufacture of a medicament for the treatment or prevention of an infection, disease or condition caused by M. catarrhalis.

48. The immunogenic fragment, immunogenic composition or vaccine according to any of claims 1-46 for use in the manufacture of a medicament for the treatment or prevention of pneumonia, otitis media and/or acute exacerbations of chronic obstructive pulmonary disease AECOPD.


SEQUENCE LISTING (90 sequences)

SEQ ID NO: 1 - protein, 630 aa, Artificial
  UspA2 protein
MKTMKLLPLKIAVTSAMIIGLGAASTANAQAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 2 - protein, 613 aa, Artificial
  UspA2 protein
MKTMKLLPLKIAVTSAMIIGLGAASTANAQSRDRSLEDIQDSISKLVQDDINTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 3 - protein, 644 aa, Artificial
  UspA2 protein
MKTMKLLPLKIAVTSALIIGLGAASTANAQQQLQTETFLPNFLSNDNYDLTDPFYHNMILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 4 - protein, 591 aa, Artificial
  UspA2 protein
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLESNVEEGLLDLSGRLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 5 - protein, 687 aa, Artificial
  UspA2 protein
MKTMKLLPLKIAVTSAMIIGLGAASTANAQQQQQQQQQQQSRTEIFFPNIFFNENHDELDDAYHNIILGDTALLDKQDGSQPQLKFYSNDKDSVPDSLLFSKLLHEQQLNGFKKGDTIIPLDKDGKPVYQVDYKLDGKGKKQKRRQVYSVTTKTATDDDVNSAYSRGILGKVDDLDDEMNFLNHDITSLYDVTANQQDAIKGLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 6 - protein, 683 aa, Artificial
  UspA2 protein
MKTMKLLPLKIAVTSAMIVGLGMASTANAQQQKSPKTETFLPNIFFNEYADDLDTLYHNMILGDTAITHDDQYKFYADDATEVPDSLFFNKILHDQLLYGFKEGDKIIPLDENGKPVYKLDKRLENGVQKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 7 - protein, 684 aa, Artificial
  UspA2 protein
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGGTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 8 - protein, 684 aa, Artificial
  UspA2 protein
MKTMKLLPLKIAVTSAMIIGLGAASTANAQQQQKTKTEVFLPNLFDNDYYDLTDPLYHSMILGDTATLFDQQDNSKSQLKFYSNDKDSVPDSLLFSKLLHEQQLNGFKAGDTIIPLDKDGKPVYTQDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLENNIEEGLLELSGHLIDQKADLTKDIKTLENNIEEGLLELSGHLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 9 - protein, 684 aa, Artificial
  UspA2 protein
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKKGDTIIPLDENGKPVYKLDSIVEQGKTKTVYSVTTKTATADDVNSAYSRGIQGDIDDLYEANKENVNRLIEHGDKIFANEESVQYLNREVQNNIENIYELVQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 10 - protein, 574 aa, Artificial
  UspA2 protein
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGHLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 11 - protein, 678 aa, Artificial
  UspA2 protein
MKTMKLLPLKIAVTSALIVGLGAASTANAQQQPQTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 12 - protein, 678 aa, Artificial
  UspA2 protein
MKTMKLLPLKIAVTSAMIVGLGAASTANAQQQQQPRTETFFPNIFFNENHDALDDVYHNMILGDTAITQDNQYKFYADAISEVPDSLLFNKILHDQQLNGFKEGDTIIPLDENGKPVYKLDEKVENGVKKSVYSVTTKTATRADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKTLENNIEEGLLELSGHLIDQKADLTKDIKALESNVEEGLLDLSGRLLDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 13 - protein, 587 aa, Artificial
  UspA2 protein
MKTMKLLPLKIAVTSALIVGLGAASTANAQLVERFFPNIFLDKPLAKQHYHNVVVGDTSIVSDLQSNSDQLKFYSDDEGLVPDSLLFNKMLHEQLLNGFKEGDTIIPLDENGKPVYKVDYKLDGKEPRKVYSVTTKIATAEDVATSSYANGIQKDIDDLYDFDHQVTERLTQHGKTIYRNGERILANEESVQYLNKEVQNNIEHIYELAQQQDQHSSDIKTLESNVEKGLLELSGHLIDQKADLTKDIKTLENNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDTNKASADTKFAATADAITKNGNAITKNAKSITDLGTKVDGFDSRVTALDTKVNALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 14 - protein, 678 aa, Artificial; identical to SEQ ID NO: 12
  UspA2 protein

SEQ ID NO: 15 - protein, 616 aa, Artificial
  UspA2 protein
MKTMKLLPLKIAVTSALIVGLGAASTANAQVRDKSLEDIEALLGKIDISKLEKEKKQQTELQKYLLLSQYANVLTMEELNKNVEKNTNSIEALGYEIGWLENDIADLEEGVEELTKNQNTLIEKDEEHDRLIAQNQADIKTLENNVVEELFNLSDRLIDQEADIAKNNASIEELYDFDNEVAERIGEIHAYTEEVNKTLEKLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 16 - protein, 676 aa, Artificial
  UspA2 protein
MKTMKLLPLKIAVTSALIVGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKALESNVEEGLLDLSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 17 - protein, 629 aa, Artificial
  UspA2 protein
MKTMKLLPLKIAVTSALIVGLGAASTANAQNGTSTKLKNLKEYAQYLDNYAQYLDDDIDDLDKEVGELSQNIAKNQANIKDLNKKLSRDIDSLREDVYDNQYEIVNNQADIEKNQDDIKELENNVGKELLNLSGRLLDQKADIDNNINNIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 18 - protein, 683 aa, Artificial
  UspA2 protein
MKTMKLLPLKIAVTSAMIVGLGMASTANAQQQRSPKTETFLPNIFFNEYADDLDTLYHNMILGDTAITHDDQYKFYADDATEVPDSLFFNKILHDQLLYGFKEGDKIIPLDENGKPVYKLDKRLDNGVQKTVYSVTTKTATADDVNSAYSRGIQGDIDDLYEANKENVNRLIEHGDKIFANEESVQYLNKEVQNNIENIHELAQQQDQHSSDIKTLKKNVEEGLLELSGHLIDQKTDIAQNQTDIQDLATYNELQDQYAQKQTEAIDALNKASSENTQNIAKNSNRIKALENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 19 - protein, 700 aa, Artificial
  UspA2 protein
MKTMKLLPLKIAVTSAMIVGLGAASTANAQAQSNRSLDQVQALLRGIDETKIKKEIQQSQQPELNKYLTFNQLANALNIEELNNNVQKNTQRLDSAATLYGDLSKTVPKSIKENKESIKENKESIKENKESIKENKESIKENKESIKENKESITTLTRKSFQNQVDIVRNNASIEDLYAYGQEVAKSIGEIHAYTEEVNKTLENLITNSVENTNNITKNKADIQALENNVVEELFNLSGRLIDQKADIDNNINNIYELAQQQDQHSSDIKTLKKNVEEGLLELSGHLIDQKADLTKDIKTLESNVEEGLLDLSGRLLDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTVIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 20 - protein, 676 aa, Artificial
  UspA2 protein
MKTMKLLPLKIAVTSALIVGLGAASTANAQATETFLPNLFDNDYIETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGHLIDQKADLTKDIKTLENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 21 - protein, 678 aa, Artificial
  UspA2 protein
MKTMKLLPLKIAVTSAMIIGLGAASTANAQLAEQFFPNIFSNHAPVKQHYHNVVVGDTSIVENLQDSDDTQLKFYSNDEYSVPDSLLFNKMLHEQQLNGFKKGDTIIPLDENGKPVYKVDYKLDGQEPRRVYSVTTKIATQDDVDNSPYSRGIQGDIDDLYEANKENVNRLIEHGDKIFANEESVQYLNKEVQNNIENIYELAQQQDQHSSDIKTLKKNVEEGLLELSGRLIDQKADIAQNQANIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 22 - protein, 613 aa, Artificial; identical to SEQ ID NO: 2
  UspA2 protein

SEQ ID NO: 23 - protein, 589 aa, Artificial
  UspA2 protein
MKTMKLLPLKIAVTSALIVGLGAASTANAQATNKDITLEDVLKSIEEIDPYELRDYIEYPTAIERFLLLSQYGNTLTLEEFDNDIELLDQDVEDLEESVTELAKNQNSLIEQGEAIKEDLQGLADFVERQEDKILQNETSIKKNTQRNLVNGFEIEKNKDAIAKNNESIEDLYDFGHEVAKSIGEIHAHNEAQNETLKDLITNSVKNTDNITKNKADIQALESNVEKGLLELSGHLIDQKADIDNNINNIHELAQQQDQHSSDIKTLKKNVEEGLLELSGHLIDQKSDIAQNQANIQDLATYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 24 - protein, 684 aa, Artificial
  UspA2 protein
MKTMKLLPLKIAVTSAMMVGLGMASTANAQQQKSPKTEIFLPNLFDNDNTELTDPLYHNMILGNTALLTQENQYKFYADDGNGVPDSLLFNKILHDQLLHGFKEGDTIIPLDENGKPVYKLDSIVEQGKTKTVYSVTTKTATADDVNSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 25 - protein, 650 aa, Artificial
  UspA2 protein
MKTMKLLPLKIAVTSALIVGLGAASTANAQQQQKTKTEVFLPNLFYNDYIEETDLLYHNMILGDTAALVDRQNYSNSQLKFYSNDEESVPDSLLFSKMLNNQQLNGFKAGDIIIPVDANGQVIYQKDTRVEGGKTRTVLSVTTKIATQQDVDSAYSRGIQGKVNDLDDEMNFLNHDITSLYDVTANQQDDIKGLKKGVKDLKKGVKGLNKELKELDKEVGVLSRDIGSLNDDVAQNNESIEDLYDFSQEVADSIGEIHAHNKAQNETLQDLITNSVENTNNITKNKADIQALENNVVEELFNLSGRLIDQKADLTKDIKTLESNVEEGLLELSGHLIDQKADIAKNQADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 26 - protein, 616 aa, Artificial
  UspA2 protein
MKTMKLLPLKIAVTSALIVGLGAVSTTNAQAQSRSLDQIQTKLADLAGKIAAGKNGGGQNNQNNQNDINKYLFLSQYANILTMEELNNNVVKNSSSIETLETDFGWLENDVADLEDGVEELTKNQNTLIEKDEEHDRLIAQNQADIQTLENNVVEELFNLSDRLIDQKADIAKNQADIAQNNESIEELYDFDNEVAEKIGEIHAYTEEVNKTLQDLITNSVKNTDNIDKNKADIDNNINHIYELAQQQDQHSSDIKTLKNNVEEGLLELSGHLIDQKADLTKDIKTLENNVEEGLLDLSGRLIDQKADIAKNQADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 27 - protein, 668 aa, Artificial
  UspA2 protein
MKTMKLLPLKIAVTSALIVGLGTASTANAQVASPANQKIQQKIKKVRKELRQDIKSLRNDIDSNTADIGSLNDDVADNQDDILDNQADIAKNQDDIEKNQADIKELDKEVGVLSREIGSLNDDIADNYTDIIDNYTDIIDNQANIAKNQDDIEKNQADIKELDKEVGVLSREIGSLNDDVADNQDDIAKNQADIQTLENNVEEGLLELSGHLLDQKADIDNNINNIYELAQQQDQHSSDIKTLKKNVEEGLLELSGHLIDQKTDIAQNQANIQDLATYNELQDQYAQEQTEAIDALNKASSENTQNIAKNSNRIKALESNVEEGLLELSGHLIDQKADLTKDIKALESNVEEGLLELSGHLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKVSADTKFAATADAITKNGNAITKNAKSITDLGTKVDAFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 28 - protein, 674 aa, Artificial
  UspA2 protein
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 29 - protein, 613 aa, Artificial
  UspA2 protein
MKTMKLLPLKIAVTSAMIIGLGAASTANAQSRDRSLEDIQDSISKLVQDDIDTLKQDQQKMNKYLLLNQLANTLITDELNNNVIKNTNSIEALGDEIGWLENDIADLEEGVEELTKNQNTLIEKDEEHDRLIAQNQADIQTLENNVVEELFNLSGRLIDQEADIAKNNASIEELYDFDNEVAERIGEIHAYTEEVNKTLENLITNSVKNTDNIDKNKADIDNNINHIYELAQQQDQHSSDIKTLKNNVEEGLLELSGHLIDQKADLTKDIKALESNVEEGLLDLSGRLLDQKADLTKDIKALESNVEEGLLDLSGRLLDQKADIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTNRIATAELGIAENKKDAQIAKAQANANKTAIDENKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 30 - protein, 576 aa, Artificial
  UspA2 protein
MKTMKLLPLKIAVTSAMIVGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVEEGLLELSGHLIDQKADLTKDIKALESNVEEGLLELSGHLIDQKADLTKDIKALESNVEEGLLDLSGRLLDQKADIAKNQADIAQNQTDIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 31 - protein, 684 aa, Artificial; identical to SEQ ID NO: 24
  UspA2 protein

SEQ ID NO: 32 - protein, 686 aa, Artificial
  UspA2 protein
MKTMKLLPLKIAVTSAMIIGLGAASTANAQATETFLPNLFDNDYTETTDPLYHGMILGNTAITQDTQYKFYAENGNEVPDSLFFNKILHDQQLNGFKEGDTIIPLDENGKPVYKLDEITENGVKRKVYSVTTKTATREDVEQSAYSRGIQGDIDDLYEANKENVNRLIEHGDKIFANEESVQYLNKEVQNNIENIHELAQQQDQHSSDIKTLKKNVEEGLLELSGRLIAQKEDIAQNQTDIQDLATYNELQDQYAQKQTEAIDALNKASSENTQNIAKNSNHIKTLENNIEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 33 - protein, 630 aa, Artificial; identical to SEQ ID NO: 1
  UspA2 protein

SEQ ID NO: 34 - protein, 616 aa, Artificial
  UspA2 protein
MKTMKLLPLKIAVTSALIVGLGAASTANAQAQDRSLEQIQDKLANLVEKIEQAKSQNGQSQKDINQYLLLSQYANVLTMEELNNNVVKNSSSIETLDNDIAWLNDDLIDLDKEVGVLSRDIGSLHDDVAQNQADIKTLKNNVVEELFNLSDRLIDQEADIAQNNESIEDLYDFGREVAESIGEIHAHNEAQNETLKDLITNSVKNTDNITKNKADIQALENDVGKELLNLSGRLIDQKADIDNNINHIYELAQQQDQHSSDIKTLKNNVEEGLLELSGHLIDQKADLTKDIKALESNVEEGLLDLSGRLLDQKADIAQNQANIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 35 - protein, 614 aa, Artificial
  UspA2 protein
MKTMKLLPLKIAVTSAMIIGLGATSTVNAQVVEQFFPNIFFNENHDELDDAYHNMILGDTAIVSNSQDNSTQLKFYSNDEDSVPDSLLFSKLLHEQQLNGFKAGDTIIPLDKDGKPVYTKDTRTKDGKVETVYSVTTKIATQDDVEQSAYSRGIQGDIDDLYDINREVNEYLKATHDYNERQTEAIDALNKASSANTDRIDTAEERIDKNEYDIKALESNVGKDLLDLSGRLIAQKEDIDNNINHIYELAQQQDQHSSDIKTLKNNVEEGLLELSGHLIDQKADLTKDIKTLESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 36 - protein, 679 aa, Artificial
  UspA2 protein
MKTMKLPPLKIAVTSAMIIGLGAASTANAQTTETFLPNLFDNDYTETTDPLYHGMILGDTAITQDTQYKFYAENGNEVPDSLFFNKILHDQLLNGFKAGDTIIPLDENGKPVYKLDERTENGVKRKVYSVTTKTATQADVEQSAYSRGIQGDIDDLYEANKENVNRLIEHGDKIFANEESVQYLNREVQNNIENIHELAQQQDQHSSDIKTLKKNVEKDLLDLSGRLIAQKEDIAQNQTDIQDLATYNELQDQYAQKQTEAIDALNKASSENTQNIAKNSNHIKTLENNIEECLLELSGHLIDQKADLTKDIKALESNVEEGLLDLSGRLIDQKADIAQNQANIQDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 37 - protein, 724 aa, Artificial
  UspA2 protein
MKTMKLLPLKIAVTSALIVGLGAASTANAQETLEEVLESIKQINEQDLQDDIGYNSALDRYLVLSQYGNLLIAKELNENVEKNSNSIAKNSNSIADLEADVGYLAENQNTLIEQNETINQELEGITHELESFIAYAHAQDQKNLVNEFEIEKNKDAIAKNNESIEDLYDFGHEVAESIGEIHAYTEEVNKTLENLITNSVKNTDNITKNKADIQALESNVEKELLNLSGRLIDQKADIDNNINHIYELAQQQDQHSSDIKTLKKNVEEGLLELSGHLIDQKSDIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 38 - protein, 611 aa, Artificial
  UspA2 protein
MKTMKLLPLKIAVTSALIVGLGAASTANAQAQARDRSLEDIQALIGNIDVDKIRSQKQKNPEIFQYLLLNQLSNTLITDELNNNVIKNTNSIETLDNDIAWLNDDLIDLDKEVGVLSRDIGSLHDDVAQNQADIKTLENNVVEELFNLSDRLIDQEAEIAQNNESIEDLYDFGREVAESIGEIHAHNEAQNETLKDLITNSVKNTDNIDKNKADIQALENNVEEGLLELSGHLIDQKADLTKDIKALESNVEEGLLDLSGRLLDQKADIAKNQADIAQNQTDIQDLAAYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKVSAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 39 - protein, 360 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFL

SEQ ID NO: 40 - protein, 348 aa, Artificial
  Haemophilus influenzae
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 41 - DNA, 989 bp, Haemophilus influenzae
gcgaatacccaaatgaaatcagacaaaatcattattgctcaccgtggtgctagcggttatttaccagagcatacgttagaatctaaagcacttgcgtttgcacaacacgcagattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgactgaaaactttgaaaccaaagacggcaaacaagcgcaagtttatcctaatcgtttcccactttggaaatcacattttagaattcacacctttgaagatgaaattgagtttatccaaggcttagaaaaatcgactggcagaaaagtagggatttatccagaaatcaaagcaccttggttccaccatcaaaatggcaaagatattgcagctgaaacgctcaaagtgttaaaaaaatatggctatgataagaaaaccgatatggtttacttacaaactttcgattttaatgaattaaaacgtatcaaaacggaattacttccacaaatgggaatggatttaaaattagttcaattaattgcttatacagattggaaagaaacacaagaaaaagacccaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgccttattgaataaatcaggggcaacaggtgtatttactgatttcccagatactgg

SEQ ID NO: 42 - DNA, 989 bp, Haemophilus influenzae; identical to SEQ ID NO: 41

SEQ ID NO: 43 - DNA, 989 bp, Haemophilus influenzae
gcaaatacccaaatgaaatctgacaaaatcatcattgctcatcgtggtgctagcggttatttaccagagcatacgttagaatctaaagcacttgcgtttgcacagcacgctgattacttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagatggcaaacagacacaagtttatcctaatcgtttccccctttggcaatcccatttccgtattcacacctttgaagatgaaattgaatttattcaaggtttagaaaaatcgacgggcaaaaaagtagggatttatccagaaatcaaagcaccttggttccaccatcaaaatggcaaagatattgctgctgaaacgctcaaagtgttaaaaaaatatggctatgataagaaaaccgatatggtttacttacaaactttcgattttaatgaattaaaacgtatcaaaacggaattacttccacaaatgggtatggatttgaaattagttcaattaattgcttatacagattggaaagaaacacaagaaaaagattcaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactacctgcgtttttcacagacgtaaatcaaatgtatgatgccttattgaataaatcaggggcaacaggtgtatttactgatttcccagatactgg

SEQ ID NO: 44 - DNA, 1259 bp, Haemophilus influenzae
acctacggtactaaataattagcttaaaaaaggcggcgggcaaattgcttagtcgccttttttgtaactaaaatctaaaaaaaaccataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggccgttactatgtcatcgactttaccttaaaagaaattcaaagtttagaaatgacagaaaactttgaaaccaaagatggcaaacaagcgcaagtttatcctaatcgtttcccactttggaaatcacattttagaattcacacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatgggtatggatttgaaattagttcaattaattgcttatacagattggaaagaaacacaagaaaaagatccaaagggttattgggtaaactataattacgattggatgtttaaacctggagcaatggcagaagtggttaaatatgccgatggtgttggtccaggttggtatatgttagttaataaagaagaatccaaacctgataatattgtgtacactccgttggtaaaagaacttgcacaatataatgtggaagtgcatccttacaccgtgcgtaaagatgcactacccgcgtttttcacagatgtaaatcaaatgtatgatgccttattgaataaatcaggggcaacaggtgtatttactgatttcccagatactggcgtggaattcttaaaaggaataaaataatatccctcacaaccgtgggtaaacatacccacgttaactagg

SEQ ID NO: 45 - DNA, 1258 bp, Haemophilus influenzae
acttacggtactaaataattagcttaaaaaaggcggtgggtaaattgcttagtcgccttttttgtaactaaaatctaaaaaaaccataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagacggcaaacaagcgcaagtttatcctaatcgtttcccactttggaaatcacattttagaattcatacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatggggatggatttgaaattagttcaattaattgcttatacagattggaaagaaacacaagaaaaagacccaaagggttattgggtaaactataattacgattggatgtttaaacctggagcaatggcagaagtggttaaatatgccgatggtgttggtccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgtcttattgaataaatcaggggcaacaggtgtatttactgatttcccagatactggcgtggaattcttaaaaggaataaaataatatccctcacaaccgtgggtaaacatacccacgttaactagg

SEQ ID NO: 46 - DNA, 1252 bp, Haemophilus influenzae
acttacggtactaaataattagcttaaaaaaggcggtgggcaaattgcttagtcgccttttttgtaactaaaatctaaaaaaaccataaaaatttaccgcactttcaaggagaaaatacttatgaaacttaaaactttagccctttctttattagcagctggcgtactagcaggttgtagcagccattcatcaaatatggcgaaaacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcaccgtaaagatggtcgttactatgtcatcgactttaccttaaaagaaattcaaagtttagaaatgacagaaaactttgaaaccaaagacggcaaacaagcgcaagtttatcctaatcgtttccccctttggcaatcccatttccgtattcacacctttgaagatgaaattgaatttatccaaggcttagaaaaatcgactggcagaaaagtagggatttatccagaaatcaaagcaccttggttccaccatcaaaatggtaaagatattgctgctgaaacgctcaaagtgttgaaaaaatagttccaccatcaaaatggtaaagatattgctgctgaaacgctcaaagtgttgaaaaaataacgtatcaaaacggaattacttccacaaatgggtatggatttgaaattagttcaattaattgcttatacagattggaaagaaacacaagaaaaagattcaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcaaaatataatgtggaagtgcatccttacaccgtgcgtaaagatgcactgcctgcgtttttcacagacgtaaatcaaatgtatgatgctttattgaataaatcaggggcaacaggtgtatttactgatttcccagatactggcgtggaattcttaaaaggaatagaataatatccctcacaaccgtgggtaaacatacccacggtt

SEQ ID NO: 47 - DNA, 1864 bp, Haemophilus influenza
gatcggcggtggcgtattagcggtgttattactcttaatcgtaatggttgaagaaggaaaacacaaagcgaaattaggcgatacttacggtactaaataattagcttaaaaaaggcggtgggcaaattgcttagtcgccttttttgtaactaaaatctaaaaactctataaaaatttaccgcactcttaaggagaaaatacttatgaaacttaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccaaagatggcaaacaagcgcaagtttatcctaatcgtttccctctttggaaatcacattttagaattcatacctttgaagatgaaattgaatttatccaaggcttagaaaaatccactggcaaaaaagtagggatttatccagaaatcaaagcaccttggttccaccatcaaaatggtaaagatattgctgctgaaacgctcaaagtgttaaaaaaatatggctatgataagaaaaccgatatggtttacttacaaactttcgattttaatgaattaaaacgtatcaaaacggaattacttccacaaatgggaatggatttgaaattagttcaattaattgcttatacagattggaaagaaacacaagaaaaagacccaaagggttattgggtaaactataattacgattggatgtttaaacctggtgcaatggcagaagtggttaaatatgccgatggtgttggcccaggttggtatatgttagttaataaagaagaatccaaacctgataatattgtgtacactccgttggtaaaagaacttgcacaatataatgtggaagtgcatccttacaccgtgcgtaaagatgcactgcccgagtttttcacagacgtaaatcaaatgtatgatgccttattgaataaatcaggggcaacaggtgtatttactgatttcccagatactggcgtggaattcttaaaaggaataaaataatatccctcacaaccgtgggtaaacatacccacggttaactaggtttctatatcgtagaaactaaaaatctactctaacagagtaacatcataatcaatctaggtgttctaacctagaattcaaataaggaggctatttcaaaacactccgtattcttttttaataaattctcttccctttacttagggaaaacactcttcatttcaaccgcacttctaaggagtgctctatggataaatcattaaaagcgaactgtattggcgagtttttaggtacagccttattgattttctttggtgtgggctgcgttgcagcactaaaagtagcaggcgctagttttggcttgtgggaaatcagcattatgtgggggatgggcgttgcacttgcagtatatgcaacagcgggtttatctggcgcacatttaaaccctgcagtaaccattgccctttggaaatttgcttgctttgatggcaaaaaagtaattccttacatcatttcacaaatgctcggcgcattctttgctgccgcattagtttatgccttataccgcaatgtttttatcgatc

SEQ ID NO: 48 - protein, 25 aa, Haemophilus influenzae
PKRYARSVRQYKILNCANYHLTQVR

SEQ ID NO: 49 - protein, 160 aa, Haemophilus influenzae
MKKIILTLSLGLLTACSAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 50 - protein, 149 aa, Haemophilus influenzae
MKLTTQQTLKKGFTLIELMIVIAIIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 51 - protein, 275 aa, Artificial
  Haemophilus influenzae
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 52 - protein, 253 aa, Artificial
  Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 53 - protein, 537 aa, Artificial
  Protein Construct from multiple organisms
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 54 - protein, 863 aa, Moraxella catarrhalis
MNKIYKVKKNAAGHLVACSEFAKGHTKKAVLGSLLIVGILGMATTASAQKVGKATNKISGGDNNTANGTYLTIGGGDYNKTKGRYSTIGGGLFNEATNEYSTIGSGGYNKAKGRYSTIGGGGYNEATNQYSTIGGGDNNTAKGRYSTIGGGGYNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAQNKADIKDLDDEVGLLGEEINSLEGEIFNNQDAIAKNQADIKTLESNVEEGLLDLSGRLLDQKADIDNNINNIYELAQQQDQHSSDIKTLKNNVEEGLLDLSGRLIDQKADLTKDIKALESNVEEGLLDLSGRLIDQKADIAKNQADIAQNQTDIQDLAAYNELQDAYAKQQTEAIDALNKASSANTDRIATAELGIAENKKDAQIAKAQANENKDGIAKNQADIQLHDKKITNLGILHSMVARAVGNNTQGVATNKADIAKNQADIANNIKNIYELAQQQDQHSSDIKTLAKVSAANTDRIAKNKAEADASFETLTKNQNTLIEQGEALVEQNKAINQELEGFAAHADVQDKQILQNQADITTNKTAIEQNINRTVANGFEIEKNKAGIATNKQELILQNDRLNRINETNNHQDQKIDQLGYALKEQGQHFNNRISAVERQTAGGIANAIAIATLPSPSRAGEHHVLFGSGYHNGQAAVSLGAAGLSDTGKSTYKIGLSWSDAGGLSGGVGGSYRWK

SEQ ID NO: 55 - protein, 815 aa, Moraxella catarrhalis
QKVGKATNKISGGDNNTANGTYLTIGGGDYNKTKGRYSTIGGGLFNEATNEYSTIGSGGYNKAKGRYSTIGGGGYNEATNQYSTIGGGDNNTAKGRYSTIGGGGYNEATIENSTVGGGGYNQAKGRNSTVAGGYNNEATGTDSTIAGGRKNQATGKGSFAAGIDNKANADNAVALGNKNTIEGENSVAIGSNNTVKKGQQNVFILGSNTDTTNAQNGSVLLGHNTAGKAATIVNSAEVGGLSLTGFAGASKTGNGTVSVGKKGKERQIVHVGAGEISDTSTDAVNGSQLHALATVVAQNKADIKDLDDEVGLLGEEINSLEGEIFNNQDAIAKNQADIKTLESNVEEGLLDLSGRLLDQKADIDNNINNIYELAQQQDQHSSDIKTLKNNVEEGLLDLSGRLIDQKADLTKDIKALESNVEEGLLDLSGRLIDQKADIAKNQADIAQNQTDIQDLAAYNELQDAYAKQQTEAIDALNKASSANTDRIATAELGIAENKKDAQIAKAQANENKDGIAKNQADIQLHDKKITNLGILHSMVARAVGNNTQGVATNKADIAKNQADIANNIKNIYELAQQQDQHSSDIKTLAKVSAANTDRIAKNKAEADASFETLTKNQNTLIEQGEALVEQNKAINQELEGFAAHADVQDKQILQNQADITTNKTAIEQNINRTVANGFEIEKNKAGIATNKQELILQNDRLNRINETNNHQDQKIDQLGYALKEQGQHFNNRISAVERQTAGGIANAIAIATLPSPSRAGEHHVLFGSGYHNGQAAVSLGAAGLSDTGKSTYKIGLSWSDAGGLSGGVGGSYRWK

SEQ ID NO: 56 - protein, 876 aa, Moraxella catarrhalis
MNKIYKVKKNAAGHLVACSEFAKGHTKKAVLGSLLIVGILGMATTASAQDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 57 - protein, 828 aa, Moraxella catarrhalis
QDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 58 - protein, 786 aa, Moraxella catarrhalis
MKTMKLLPLKIAVTSAMIVGLGAASNANAQTSNSGTNQIVEEFLPKLTQALNDPQYQHEFEDALDQYHNMVVGNTSFVSTVDVANQTVGNTALKFYSDSENTLPSSMLFDQILTNQRLNGFKEGQPFIPVDDNGNPITQVDDVLESNGQTKKVRSVTTRIATAEDVTKSPYAQGIQKDIDDIYEFNHEVVDNIATLNRATEILTEAGNHHDELIRKNKADIQVSNGKIESLKTLYETTVRTVGNNESNIKTNKDAIAKNKANIKTLAQAQLESVTFLGEQIQENKDDISAVTGKAFKNQIDIQRNNDSIEDLYEANNDNVERLVEHDRAIEILAEAGNHHDTLIRKNKADIQDLAAHNELQTEAIDALNKASSANTKRITTAELGIAENKKDALIAKAQADKNKNDIQDLAKVQRAGVEVMAELNKNIAGTQTDIANNKITLAQHAKKIDENQQAIEAKLGGKADLQKLSELKTSVDKNQAGIQLHDQKINNLGILHTMVARAVGANKTAIDQNKADIAENKEDIQNNINNIYELAQQQDQHSSDIKTLAKASSANTDNIAKNKADADASFKTLTKNQNTLIEKDKAQDALITANKADADASFKTLTKNQNTLIEKDKAQDKLITANKADADAKFTGVQTDIANNKTTLAQHAKKINDNQQAIEAKLGGKADAQALNNLNDKVNGFDGRISALDTKVNAFDGRITALDSKVENGMAAQAALSGLFQPYSVGKFNATAALGGYGSKSAVAIGAGYRVNPNLAIKAGAAINTSGDKKGSYNIGVNYEF

SEQ ID NO: 59 - protein, 757 aa, Moraxella catarrhalis
QTSNSGTNQIVEEFLPKLTQALNDPQYQHEFEDALDQYHNMVVGNTSFVSTVDVANQTVGNTALKFYSDSENTLPSSMLFDQILTNQRLNGFKEGQPFIPVDDNGNPITQVDDVLESNGQTKKVRSVTTRIATAEDVTKSPYAQGIQKDIDDIYEFNHEVVDNIATLNRATEILTEAGNHHDELIRKNKADIQVSNGKIESLKTLYETTVRTVGNNESNIKTNKDAIAKNKANIKTLAQAQLESVTFLGEQIQENKDDISAVTGKAFKNQIDIQRNNDSIEDLYEANNDNVERLVEHDRAIEILAEAGNHHDTLIRKNKADIQDLAAHNELQTEAIDALNKASSANTKRITTAELGIAENKKDALIAKAQADKNKNDIQDLAKVQRAGVEVMAELNKNIAGTQTDIANNKITLAQHAKKIDENQQAIEAKLGGKADLQKLSELKTSVDKNQAGIQLHDQKINNLGILHTMVARAVGANKTAIDQNKADIAENKEDIQNNINNIYELAQQQDQHSSDIKTLAKASSANTDNIAKNKADADASFKTLTKNQNTLIEKDKAQDALITANKADADASFKTLTKNQNTLIEKDKAQDKLITANKADADAKFTGVQTDIANNKTTLAQHAKKINDNQQAIEAKLGGKADAQALNNLNDKVNGFDGRISALDTKVNAFDGRITALDSKVENGMAAQAALSGLFQPYSVGKFNATAALGGYGSKSAVAIGAGYRVNPNLAIKAGAAINTSGDKKGSYNIGVNYEF

SEQ ID NO: 60 - protein, 14 aa, Artificial
  Moraxella catarrhalis
YNELQDAYAQKQTE

SEQ ID NO: 61 - protein, 14 aa, Artificial
  Moraxella catarrhalis
YNELQDAYAKQQTE

SEQ ID NO: 62 - protein, 14 aa, Artificial
  Moraxella catarrhalis
YNELQDQYAQKQTE

SEQ ID NO: 63 - protein, 14 aa, Artificial
  Moraxella catarrhalis
YNELQDQYAKQQTE

SEQ ID NO: 64 - protein, 17 aa, Artificial
  Moraxella catarrhalis
YNELQDAQYAQKKQQTE

SEQ ID NO: 65 - protein, 141 aa, Artificial
  Haemophilus influenzae
IQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKK

SEQ ID NO: 66 - protein, 110 aa, Artificial
  Haemophilus influenzae
TKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 67 - protein, 10 aa, Artificial
  Haemophilus influenzae
SSHSSNMANT

SEQ ID NO: 68 - protein, 894 aa, Moraxella catarrhalis
MNKIYKVKKNAAGHSVACSEFAKGHTKKAVLGSLLIVGALGMATTASAQTGSTNAANGNIISGVGAYVGGGVINQAKGNYPTVGGGFDNRATGNYSVISGGFDNQAKGEHSTIAGGESNQATGRNSTVAGGSNNQAVGTNSTVAGGSNNQAKGANSFAAGVGNQANTDNAVALGKNNTINGNNSAAIGSENTVNENQKNVFILGSNTTNAQSGSVLLGHETSGKEATAVSRARVNGLTLKNFSGVSKADNGTVSVGSQGKERQIVHVGAGQISDDSTDAVNGSQLYALATAVDDNQYDIEINQDNIKDLQKEVKGLDKEVGVLSRDIGSLHDDVADNQADIAKNKADIKELDKEMNVLSRDIVSLNDDVADNQADIAKNQADIKTLENNVEEGLLDLSGRLIDQKADIDNNINHIYELAQQQDQHSSDIKTLKKNVEEGLLELSGHLIDQKADIAQNQTDIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIEDLAAYNELQDAYAKQQTEAIDALNKASSENTQNIQDLAAYNELQDAYAKQQTEAIDALNKASSENTQNIQDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 69 - protein, 877 aa, Moraxella catarrhalis
MNKIYKVKKNAAGHSVACSEFAKGHTKKAVLGSLLIVGALGMATTASAQLVSTTQPNDYRSSTTGNNHLGSSWSIIGAGHDNIVYRSASNSGILSGYKNRVNGSTSAIVGGYDNETRGKYTFVGGGYKNLAEGHQSAIGGGYANWAEGDNATIAGGFENFAAGNQSAIGGGYANLAEGDDATIAGGFENRAEGNQSAIGGGYANFAAGDYTFVGGGYENRAEGNQSAIGGGYANLAEGDNATIAGGFENRAKGINSVVSGGYANQATGESSTIAGGFENRAEGIDSVVSGGYANQANGAQSTVAGGYNNQATGESSTIAGGSNNQATGTGSFAAGVENKANADNAVALGKNNIINGDNSAAIGSNNTVKKGQKDVFILGSNTSGAQSNSVLLGNETTGKKATAVENATVGDLSLTGFAGVSKANSGTVSVGSEGKERQIVHVGAGRISNDSTDAVNGSQLYALAAAVDDNQYDIEKNQDDIKELKRGVKELDKEMNVLSRDIVSLNDDVAQNQSDIKTLKNNVEEGLLELSGHLIDQKADLTKDIKALENNVEEGLLDLSGRLLDQKADIAKNQADIAQNQTDIQDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 70 - protein, 861 aa, Moraxella catarrhalis
VNKIYKVKKNVAGHSVACSEFAKGHTKKAVLGSLLIVGALGMATTASAQQNNKGFGAYIGVENSGNLATGQYSVAVGGLGNEATGQYSVINGGYFNIADGVHSIIGGGKDNKAKGNYSTIGGGDSNKAEGNYSTIAGGRNNEATGEGSFAAGIDNKANAKNAVALGNKNSIEGTNSVAIGSNNTVAQDNTFILGSNTTGVQRNSVLLGNNTAGQEATIVEKAEVGGVNLTGFAGVSKTSNGTVSVGSKNNERQIVNVGAGRISKDSTDAVNGSQLYALATAVDDSQYDIEKNQDDIEKNQDDIKELKRGVKDLKKGVKGLDKEVKDLNKKVGVLDRDIGSLQDDVADNQADIANNQADIKDLDKEVGVLSRDIGSLHDDVAQNQSDIKTLKNNVEEGLLELSGHLLDQKADIDNNINNIYELAQQQDQHSSDIKTLKKNVEEGLLELSGHLIDQKADIAQNQTDIQDLATYNELQDQYAQKQTEAIDALNKASSENTQNIAKNSNRIKALESNVEEGLLELSGHLIDQKADLTKDIKALESNVEEGLLDLSGRLIDQKADIAQNQANIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 71 - protein, 875 aa, Moraxella catarrhalis
VNKIYKVKKNVAGHSVACSEFAKGHTKKAVLGSLLIAGALGMATTASAQVVGNTTEGLGARVNGGDGNEAKGTYPTIGGGFFNIATGNYSTISGGNTNEAEGNYSTIGGGDTNAASGNNSTVVGGYNNTAKGNYSTVAGGANNQANSIDSTIVGGRKNRAKGKSSTVSGGYDNLAGGKSSAIGGGEFNSAIGSRSTVAGGELNLASGDNATVSGGKQNQAIGEYSTVAGGELNLASGDNATVSGGKQNQAEGTDSTIAGGRNNQAIGEGSFAAGIDNKANTENAVAVGKKNIINGDNSAAIGSNNTVKKGQTGVFILGSNTKGAQSNSVLLGNETTGKKATAVSSATVNGLTLENFAGVSQVGNGTVSVGSKGKERQIVNVGAGKISATSTDAVNGSQLHALATAVAYNYTDIIDNQADIAKNQDDIDDLNKKVGVLDRDIGSLQDDVADNQADIANNQADIKDLDDEVGVLSRDIGSLHDDVAQNQSDIKTLKNNVEEGLLDLSGRLLDQKADIDNNINNIYELAQQQDQHSSDIKTLKKNVEEGLLELSGHLIDQKSDIAKNQADIAQNQTDIHDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDAFDGRVTALDTKVNALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 72 - protein, 876 aa, Moraxella catarrhalis
VNKIYKVKKNAAGHLVACSEFAKGHTKKAVLGSLLIVGILGMATTASAQDRAGNQRNGAHSAIGGGSINIADGDYSTIGGGHINTTKGNYSTIGGGNENKTTGNYSTIGGGYGNEAEGKHSTIGGGDENKAKGDYSTIGGGDDNEATGTHSTIGGGDDNKATGDYSIIGGGYGNRATAEDATVSGGGYNQAAGKSSTVSGGYDNLAEGESSAIGGGELNSTIGSHSTVAGGFLNGAVGDNATVSGGEQNQAIGKYSTVSGGYGNQATGIGSFAAGIENQANTENAVAVGKKNIINGDNSAAIGSNNTVEKGQKDVFILGSNTKDAQSNSVLLGNETTGKAATVVDSANVGGLGLTGFAGVSKVGNGTVSVGSQGKERQIVNVGAGEISATSTDAVNGSQLHALATAVDNNQYDIINNQGDIERNQDDIKDLQKEVKGLDNEVGELSRDINSLHDVTDNQQDDIKELKRGVKELDKEMNVLSRDIVSLNDDVADNQADIAKNQADIKTLESNVEEGLLDLSGRLLDQKADIANNVNHIYELAQQQDQHSSDIKTLKKNVEEGLLELSGHLIDQKSDIAQNQTDIQDLAAYNELQDQYAQKQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 73 - protein, 655 aa, Moraxella catarrhalis
MKTMKLLPLKIAVTSALIVGLGAASTANAQKKASSQELNQHIRVLNKYIRDFNDDIQELDYDIQDLNDDIKGLDKKLSRDINSLRDEVTDNYTDIIDNQVDIERNQADIKDLKRGVKGLNKEVGVLDRDIGSLQDDVADNQADIANNQDDIKDLDDEVGVLSRDIGSLHDDVAQNQSDIKTLESNVEEGLLELSGHLIDQKADIDNNINHIYELAQQQDQHSSDIKTLKKNVEEGLLELSGHLIDQKSDIAQNQTDIQDLATYNELQDQYAQKQTEAIDALNKASSENTQNIQDLAAYNELQDAYAKQQTEAIDALNKASSENTQNIEDLAAYNELQDQYAQKQTEAIDALNKASSENTQNIQDLAAYNELQDAYAKQQTEAIDALNKASSENTQNIEDLAAYNELQDQYAQKQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDGRVTALDTKVNAFDGRITALDSKVENGMAAQAALSGLFQPYSVGKFNATAALGGYGSKSAVAIGAGYRVNPNLAFKAGAAINTSGNKKGSYNIGVNYEF

SEQ ID NO: 74 - protein, 591 aa, Moraxella catarrhalis; identical to SEQ ID NO: 4

SEQ ID NO: 75 - protein, 687 aa, Moraxella catarrhalis; identical to SEQ ID NO: 5

SEQ ID NO: 76 - protein, 31 aa, Artificial
  Motifs
  misc_feature (2)..(3): Xaa can be any naturally occurring amino acid
  misc_feature (6)..(7): Xaa can be any naturally occurring amino acid
  misc_feature (9)..(10): Xaa can be any naturally occurring amino acid
  misc_feature (13)..(14): Xaa can be any naturally occurring amino acid
  misc_feature (16)..(17): Xaa can be any naturally occurring amino acid
  misc_feature (19)..(21): Xaa can be any naturally occurring amino acid
  misc_feature (26)..(27): Xaa can be any naturally occurring amino acid
  misc_feature (29)..(31): Xaa can be any naturally occurring amino acid
NXXQDXXSXXNTXXQXXHXXXANDQXXDXXX

SEQ ID NO: 77 - protein, 7 aa, Artificial
  Motifs
  misc_feature (2)..(3): Xaa can be any naturally occurring amino acid
  misc_feature (6)..(7): Xaa can be any naturally occurring amino acid
SXXNTXX

SEQ ID NO: 78 - protein, 7 aa, Artificial
  Motifs
  misc_feature (2)..(3): Xaa can be any naturally occurring amino acid
  misc_feature (5)..(7): Xaa can be any naturally occurring amino acid
QXXHXXX

SEQ ID NO: 79 - protein, 7 aa, Artificial
  Motifs
  misc_feature (2)..(3): Xaa can be any naturally occurring amino acid
  misc_feature (5)..(7): Xaa can be any naturally occurring amino acid
QXXDXXX

SEQ ID NO: 80 - protein, 25 aa, Artificial
  Moraxella catarrhalis
DLAAYNELQDAQYAKQQKQTEAIDA

SEQ ID NO: 81 - DNA, 349 bp, Artificial
  Antibody Variable Heavy Chain
gaggttcagctccagcagtctgggactgtgctggcaaggcctggggcttccgtgaagatgtcctgcaaggcttctggctacagctttaccagctactggatgcactgggtaaaacagaggcctggacagggtctagaatggattggtgctatttatcctggaaatagtgatactagctacaaccagaagttcaagggcaaggccaaactgactgcagtcacatccgccagcactgcctacatggagctcagcaacctgacgaatgaggactctgcggtctattcctgtacattactacgtttcctcgatgcttactggggccaagggactctggtcactgtctctgcag

SEQ ID NO: 82 - protein, 116 aa, Artificial
  Antibody Variable Heavy Chain
EVQLQQSGTVLARPGASVKMSCKASGYSFTSYWMHWVKQRPGQGLEWIGAIYPGNSDTSYNQKFKGKAKLTAVTSASTAYMELSNLTNEDSAVYSCTLLRFLDAYWGQGTLVTVSA

SEQ ID NO: 83 - DNA, 318 bp, Artificial
  Antibody Variable Light Chain
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccacccacgttcggaggggggtccaagctggaaataaaa

SEQ ID NO: 84 - protein, 106 aa, Artificial
  Antibody Variable Light Chain
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGSKLEIK

SEQ ID NO: 85 - protein, 5 aa, Artificial
  antibody regions
SYWMH

SEQ ID NO: 86 - protein, 17 aa, Artificial
  antibody regions
AIYPGNSDTSYNQKFKG

SEQ ID NO: 87 - protein, 7 aa, Artificial
  antibody regions
LRFLDAY

SEQ ID NO: 88 - protein, 10 aa, Artificial
  antibody regions
RASSSVSYMH

SEQ ID NO: 89 - protein, 7 aa, Artificial
  antibody regions
ATSNLAS

SEQ ID NO: 90 - protein, 9 aa, Artificial
  antibody regions
QQWSSNPPT